(19) United States
(12) Patent Application Publication
(10) Pub. No.: US 2008/0125454 A1
(43) Pub. Date: May 29, 2008

(54) Methods and Compositions for Treatment of Scleritis and Related Disorders

(75) Inventors: Patricia W. Bedard, Foxboro, MA (US); Robert G. Schaub, Pelham, NH (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/868,052

(22) Filed: Oct. 5, 2007

Related U.S. Application Data
(60) Provisional application No. 60/849,580, filed on Oct. 5, 2006.

(51) Int. Cl. A61K 31/4375; A61K 31/437; A61K 31/435; A61P 27/02

(52) U.S. Cl. 514/292; 514/290

(57) ABSTRACT

The present teachings relate to the field of anti-inflammatory substances and more particularly to compounds that are useful for the treatment of scleritis, a scleritis symptom, or a scleritis-related disorder. In one aspect, methods of treating scleritis, a scleritis symptom, or a scleritis-related disorder generally include administering to a subject a compound of Formula I:

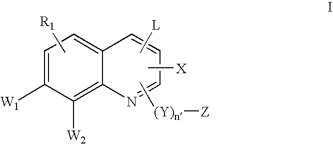

or a pharmaceutically acceptable salt, hydrate or ester thereof, wherein W₁, W₂, R₁, L, X, Y, Z, and n¹ are defined as described herein.

### BACKGROUND OF THE INVENTION

The present teachings relate to methods and compositions for treatment of scleritis, scleritis symptoms, or a scleritis-related disorder.

During the initial phase of vascular inflammation, leukocytes and platelets in flowing blood decrease velocity by adhering to the vascular endothelium and by exhibiting rolling behavior. This molecular tethering event is mediated by specific binding of a family of calcium dependent or “C-type” lectins, known as selecting, to ligands on the surface of leukocytes.

Three human selectin proteins have been identified, including P-selectin (formerly known as PADGEM or GMP-140), E-selectin (formerly known as ELAM-1), and L-selectin (formerly known as LAM-1). E-selectin expression is induced on endothelial cells by proinflammatory cytokines via its transcriptional activation. L-selectin is constitutively expressed on leukocytes and appears to play a key role in lymphocyte homing. P-selectin is stored in the alpha granules of platelets and the Weibel-Palade bodies of endothelial cells and therefore can be rapidly expressed on the surface of these cell types in response to proinflammatory stimuli.

Several diseases and disorders can cause the deleterious triggering of selectin-mediated cellular adhesion. Scleritis is believed to be one such disorder. Scleritis is an inflammation of the sclera, which is the white outer wall of the eye that forms the white of an eye. Scleritis often causes red or pink eye, tears, pain and sensitivity of the eye, and blurred vision. Left untreated, scleritis is a vision-threatening condition that can lead to permanent visual impairment.

Although the specific cause of scleritis is unknown, autoimmunity disorders are believed to be the most common cause. To treat the inflammation, patients suffering from scleritis often are given nonsteroidal anti-inflammatory drugs (NSAIDS) such as ibuprofen or steroidal compounds, for example, cortisone-related drugs. These drugs have unwanted side effects particularly when multiple doses are administered over time. However, because the adhesion of leukocytes to the vascular endothelium is a step in developing an inflammatory response, interfering with the selectin-mediated cell adhesion process can provide a new type of treatment for conditions such as scleritis or scleritis-related disorders. (See Sangwan, V. S. et al., Arch. Opthalmol. 116: 1476-1480 (1998)). Accordingly, there is a continuing need for new compounds that can be used to treat scleritis, scleritis symptoms, and/or scleritis-related disorders.

### SUMMARY OF THE INVENTION

The present teachings provide compounds and methods for treating scleritis, scleritis symptoms, and/or scleritis-related disorders. In one aspect, the present teachings provide compounds useful in the methods, where the compounds have the Formula I:

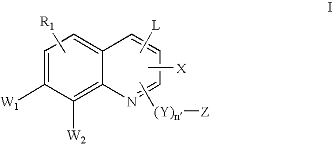

or a pharmaceutically acceptable salt, hydrate or ester thereof, wherein R₁, L, X, Y, Z, W₁, W₂, and n are as defined herein.

In various embodiments, the compounds have the Formula II, III or IV, including their pharmaceutically acceptable salts, hydrates and esters.

Also provided in accordance with the present teachings are pharmaceutical compositions for treating scleritis, scleritis symptoms, and/or scleritis-related disorders, the pharmaceutical composition comprising a therapeutically effective amount of a compound of the present teachings, and a pharmaceutically acceptable carrier or excipient.

The present teachings also provide methods for using the compounds disclosed herein. In some embodiments, the present teachings provide methods of treating scleritis, a scleritis symptom or a scleritis-related disorder, where the method generally comprises administering to a subject a therapeutically effective amount of a compound of the present teachings. In certain embodiments, the subject is a mammal, for example, a human.

Additionally, the present teachings provide for a method for reducing and/or preventing leukocyte adhesion to the vascular endothelium.

### BRIEF DESCRIPTION OF THE DRAWINGS

FIG. 1 is a graph depicting leukocyte rolling flux (measured in rolling cells per minute) in cremaster postcapillary venules in C57 black/6J mice after oral dosing with vehicle, neutralizing anti-mouse P-selectin (CD62P) antibody or Compound 1 at dosages of 10 and 50 mg/kg.

FIG. 2 is a graph depicting leukocyte rolling flux (measured in rolling cells per minute) in cremaster postcapillary venules in Sprague-Dawley rats after oral dosing with vehicle, neutralizing anti-rat PSGL-1 antibody or Compound 1 at dosages of 30 and 50 mg/kg.

### DETAILED DESCRIPTION

The present teachings provide methods and compounds for treating scleritis, a scleritis symptom, and/or a scleritis-related disorder. Without wishing to be bound to any particular theory, it is believed that interfering or preventing selectin-mediated intercellular adhesion can be useful both in the treatment of scleritis or a scleritis-related disorder, as well as for ameliorating one or more symptoms of such disease or disorder.

In various embodiments, the methods include administering to a mammal a compound of Formula I, Formula II, Formula III, Formula IV, or a pharmaceutically acceptable salt, hydrate or ester thereof; or a pharmaceutical composition comprising a compound of Formula I, Formula II, Formula III or Formula IV, or a pharmaceutically acceptable salt, hydrate or ester thereof, and a pharmaceutically acceptable carrier or excipient.

In some embodiments, methods of the present teachings comprise a method for treating scleritis, a symptom of scleritis, or a scleritis-related disorder comprising administering to a subject a therapeutically effective amount of one or more compounds having the Formula I:

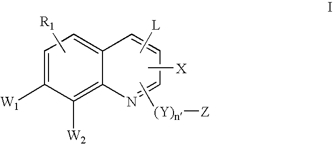

wherein:
W₁ and W₂ taken together with the atoms to which they are attached form a 5 member carbocyclic or heterocyclic ring or a 6 member carbocyclic or heterocyclic ring, any of which can be saturated, partially saturated or aromatic, and that can be substituted with up to three groups independently selected from hydrogen, C₁₋₆ alkyl, C₁₋₆ perhaloalkyl, OC₁₋₆ alkyl, OC₁₋₆ perhaloalkyl, halogen, C₁₋₆thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, C₆₋₁₄ aryl, 3 to 14 membered heterocyclo, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, and NHCOR₈, wherein any of said C₁₋₆ alkyl, OC₁₋₆alkyl, C₆₋₁₄ aryl, 3 to 14 membered heterocyclo, C(═O)C₆₋₁₄aryl, 3 to 14 membered C(═O)heterocyclo, O—C(═O)C₆₋₁₄aryl, 3 to 14 membered O—C(═O)heterocyclo, OC₆₋₁₄aryl, 3 to 14 membered O-heterocyclo, C₇₋₂₄ arylalkyl, C(═O)C₇₋₂₄ arylalkyl, O—C(═O)C₇₋₂₄ arylalkyl, O—C₇₋₂₄ arylalkyl, C₂₋₂₀ alkenyl or C₂₋₂₀alkynyl can optionally be substituted with up to three substituents independently selected from halogen, C₁₋₆ alkyl, OC₁₋₆ alkyl and CN;
L is CO₂H, an ester thereof, or a pharmaceutically acceptable acid mimetic;
Y is O, (CR₃R₄)ₚ or NR₅;
n′ is 0 or 1;
p is 1, 2, or 3
X is hydrogen, OH, OR₃, OC₁₋₆ alkyl, OC(═O)C₆₋₁₄ aryl, OC(═O)C₁₋₆ alkyl, OC(═O)OC₁₋₆ alkyl, or NR₃R₃;
   each R₁, R₃, R_{3′} and R₄ is independently hydrogen, C₁₋₆ alkyl, C₁₋₆ perhaloalkyl, OC₁₋₆ alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, OSO₃R₆, SO₃R₆, PO₃R₆R₇, (CH₂)ₙSO₂NR₈R₉, (CH₂)ₙC(═O)NR₈R₉, NR₈R₉, C(═O)R₁₂, C₆₋₁₄ aryl, 3 to 14 membered heterocyclo, C(═O)C₆₋₁₄aryl, 3 to 14 membered C(═O)heterocyclo, OC(═O)C₆₋₁₄ aryl, 3-14 membered OC(═O)heterocyclo, OC₆₋₁₄ aryl, 3-14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, or NHCOR₈, wherein any of said C₁₋₆alkyl, OC₁₋₆alkyl, C₆₋₁₄aryl, 3-14 membered heterocyclo, C(═O)C₆₋₁₄ aryl, 3-14 membered C(═O)heterocyclo, O—C(═O)C₆₋₁₄aryl, 3-14 membered O—C(═O)heterocyclo, OC₆₋₁₄aryl, 3-14membered O-heterocyclo, C₇₋₂₄ arylalkyl, C(═O)C₇₋₂₄ arylalkyl, O—C(═O)C₇₋₂₄ arylalkyl, O—C₇₋₂₄ arylalkyl, C₂₋₂₀ alkenyl or C₂₋₂₀alkynyl can be optionally substituted with up to three substituents independently selected from halogen, C₁₋₆ alkyl, OC₁₋₆ alkyl and CN;
   each R₆ and R₇ is independently hydrogen or C₁₋₆ alkyl that can be optionally substituted with up to three substituents independently selected from OH, CF₃, SH and halogen;
   each R₅, R₈ and R₉ is independently hydrogen, C₁₋₆ alkyl, C₁₋₆ haloalkyl, C₁₋₆ thioalkyl, OH, (CH₂)_{I}OSO₃H, (CH₂)_{I}SO₃R₁₀, (CH₂)ₙCO₂R₁₀, SO₃R₁₀, PO₃R₁₀R₁₁, (CH₂)ₙSO₂(CH₂)ₙNR₁₀R₁₁, (CH₂)ₙCONR₁₀R₁₁, COR₁₀, C₆₋₁₄ aryl, 3 to 14 membered heterocyclo, C(═O)C₆₋₁₄aryl, 3-14 membdered C(═O)heterocyclo, OC(═O)C₆₋₁₄aryl, 3-14membered OC(═O)heterocyclo, OC₆₋₁₄aryl, 3-14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, or C₂₋₂₀ alkynyl, wherein any of said C₁₋₆ alkyl, C₆₋₁₄ aryl, 3-14 membered heterocyclo, C(═O)C₆₋₁₄ aryl, 3-14 membered C(═O)heterocyclo, OC(═O)C₆₋₁₄ aryl, 3-14 membered OC(═O)heterocyclo, OC₆₋₁₄aryl, 3-14membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀alkenyl or C₂₋₂₀ alkynyl can optionally be substituted with up to three substituents independently selected from halogen, C₁₋₆ alkyl, OC₁₋₆ alkyl and CN;
      each n is an independently selected integer from 0 to 6;
      each I is an independently selected integer from 1 to 6;
      each R₁₀ and R₁₁ is independently hydrogen or C₁₋₆ alkyl that is optionally substituted with up to three substituents independently selected from OH, CF₃, SH and halogen;
      each R₁₂ is independently hydrogen, C₁₋₆ alkyl, C₁₋₆ perhaloalkyl, OC₁₋₆ alkyl, OC₁₋₆ perhaloalkyl, C₁₋₆ thioalkyl, OH, (CH₂)_{I}OSO₃H, (CH₂)_{I}SO₃H, (CH₂)_{I}CO₂R₆, (CH₂)_{I}SO₂NR₈R₉, (CH₂)_{I}C(═O)NR₈R₉, NR₈R₉, C₂₋₂₀ alkenyl, C₂₋₂₀ alkynyl, or NHCOR₈, wherein any of said C₁₋₆ alkyl, OC₁₋₆ alkyl, C₂₋₂₀ alkenyl or C₂₋₂₀ alkynyl can optionally be substituted with up to three substituents independently selected from halogen, C₁₋₆ alkyl, OC₁₋₆ alkyl and CN; and
      Z is C₆₋₁₄ aryl, 5 to 13 membered heteroaryl, C₇₋₂₄ arylalkyl or 3 to 14 membered heterocyclo, wherein each of said C₆₋₁₄ aryl, 5 to 13 membered heteroaryl, C₇₋₂₄ arylalkyl and 3 to 14 membered heterocyclo can be optionally substituted.

In some embodiments of Formula I, W₁ and W₂ taken together with the atoms to which they are attached form a 5 member or 6 member heterocyclic ring that can be saturated, partially saturated or aromatic, and is optionally substituted as described herein. The heterocyclic ring can have up to 3 or 4 heteroatoms, in which the heteroatom or heteroatoms are independently selected from O, N, S and NR₁₃, such as, for example, pyrrolidine, pyrroline, tetrahydrothiophene, dihydrothiophene, tetrahydrofuran, dihydrofuran, imidazoline, tetrahydroimidazole, dihydropyrazole, tetrahydropyrazole, oxazoline, piperidine, dihydropyridine, tetrahydropyridine, dihydropyran, tetrahydropyran, dioxane, piperazine, dihydropyrimidine, tetrahydropyrimidine, morpholine, thioxane, thiomorpholine, pyrrole, furan, thiophene, pyrazole, imidazole, oxazole, oxadiazole, isoxazole, thiazole, thiadiazole, isothiazole, pyridine, pyrimidine, pyrazine, pyran and triazine.

It should be noted that wherein W₁ and W₂ taken together with the atoms to which they are attached form a saturated ring, such as a piperidine ring, it is understood that the bond between W₁ and W₂ remains unsaturated.

In various embodiments, methods of the present teachings comprise administering one or more compounds having the Formula II:

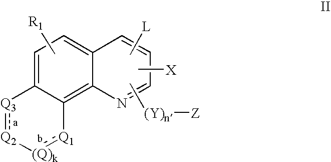

wherein:
bond a and bond b can each independently be a single bond or a double bond;
Q₁, Q₂, Q₃ and Q are each independently CR_{2′}, CHR_{2′}, N or NR₁₃;
k is 0 or 1;
each R_{2′} is independently hydrogen, C₁₋₆ alkyl, C₁₋₆ perhaloalkyl, OC₁₋₆ alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, SO₃, SO₃R₆, PO₃R₆R₇, (CH₂)ₙSO₂NR₈R₉, (CH₂)ₙC(═O)NR₈R₉, NR₈R₉, C(═O)R₁₂, C₆₋₁₄ aryl, 3 to 14 membered heterocyclo, C(═O)C₆₋₁₄aryl, 3-14 membered C(═O)heterocyclo, OC(═O)C₆₋₁₄ aryl, 3-14 membered OC(═O)heterocyclo, OC₆₋₁₄aryl, 3-14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₀ alkynyl, or NHCOR₈, wherein any of said C₁₋₆ alkyl, OC₁₋₆alkyl, C₆₋₁₄ aryl, 3-14 membered heterocyclo, C(═O)C₆₋₁₄ aryl, 3-14 membered C(═O)heterocyclo, O—C(═O)C₆₋₁₄aryl, 3-14 membered O—C(═O)heterocyclo, OC₆₋₁₄aryl, 3-14 membered O-heterocyclo, C₇₋₂₄ arylalkyl, C(═O)C₇₋₂₄ arylalkyl, O—C(═O)C₇₋₂₄ arylalkyl, O—C₇₋₂₄ arylalkyl, C₂₋₂₀ alkenyl or C₂₋₂₀ alkynyl can optionally be substituted with up to three substituents independently selected from halogen, C₁₋₆ alkyl, C₁₋₆ alkyl and CN; and
each R₁₃ is independently hydrogen, C(═O)R₂₀, SO₂R₂₀, C₁₋₆ alkyl, C₁₋₆ haloalkyl, C₁₋₆ thioalkyl, OH, (CH₂)_{I}OSO₃H, (CH₂)_{I}SO₃R₁₀, (CH₂)ₙCO₂R₁₀, SO₃R₁₀, PO₃R₁₀R₁₁, (CH₂)ₙSO₂(CH₂)ₙNR₁₀R₁₁, (CH₂)ₙCONR₁₀R₁₁, COR₁₀, C₆₋₁₄ aryl, 3 to 14 membered heterocyclo, C(═O)C₆₋₁₄aryl, 3-14 membered C(═O)heterocyclo, OC(═O)C₆₋₁₄ aryl, 3-14 membered OC(═O)heterocyclo, OC₆₋₁₄aryl, 3-14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, or C₂₋₂₀ alkynyl, wherein any of said C₁₋₆alkyl, C₆₋₁₄aryl, 3-14 membered heterocyclo, C(═O)C₆₋₁₄aryl, 3-14 membered C(═O)heterocyclo, OC(═O)C₆₋₁₄aryl, 3-14 membered OC(═O)heterocyclo, OC₆₋₁₄ aryl, 3-14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl or C₂₋₂₀ alkynyl can optionally be substituted with up to three substituents independently selected from halogen, C₁₋₆ alkyl, C₁₋₆ alkyl and CN;
each R₂₀ is independently C₁₋₁₀ alkyl, OC₁₋₁₀ alkyl or NR₆R₇; and
R₁, L, X, Y, n′, and Z are as described above.

In some embodiments, Y is CR₃R₄, for example, CH₂. In other embodiments, Y is CH₂ and X is OH. In other embodiments, Y is CH₂, X is OH and Z is C₆₋₁₄ aryl, for example, phenyl or a substituted phenyl. In some embodiments, Z is phenyl substituted at the 4′-position. In some embodiments, such 4′-substitutents are groups such as, for example, halogens, C₁₋₆ alkyl, C₁₋₆ perhaloalkyl, OC₁₋₆ alkyl, OC₁₋₆ perhaloalkyl, C₁₋₆ thioalkyl, CN, alklysulfonamides, or mono- and di-alkylamines.

In some embodiments, but not limited to those wherein Y is CH₂, X is OH, and Z is phenyl or a substituted phenyl as described above, R₁ is a group such as, for example, halogen, C₁₋₆ alkyl, C₁₋₆ perhaloalkyl, OC₁₋₆ alkyl, OC₁₋₆ perhaloalkyl, C₁₋₆ thioalkyl, CN, C₁₋₆ alkylsulfonamides, C₁₋₆ mono- and di-alkylamines, C₆₋₁₄ aryl, or a substituted C₆₋₁₄ aryl, wherein the substituents are independently selected from halogen, C₁₋₁₀ alkyl, OC₁₋₁₀ alkyl, CHO, CO₂H, NO₂, NH₂, CN, CF₃ and OH.

In various other embodiments, methods of the present teachings comprise administering one or more compounds where substituents (Y)_{n′}-Z, X and L are attached at the 2-, 3- and 4-positions of the quinoline, respectively, as shown below in Formula III:

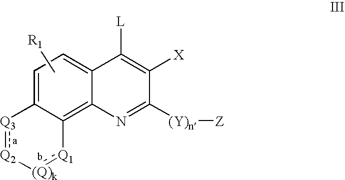

In some embodiments, k is 1, and bonds a and b are each single bonds. In certain embodiments, k is 1, bonds a and b are each single bonds, and Q, Q₁, Q₂ and Q₃ are each independently CHR_{2′}, for example, CH₂.

In certain embodiments, k is 0, bond a is a single bond, and Q₁, Q₂ and Q₃ are each independently CHR_{2′}, for example, CH₂.

In some embodiments, k is 0, bond a is a single bond, Q₁ is NR₁₃, for example, NH, and Q₂ and Q₃ are each CH₂.

In certain embodiments, k is 1, bond a and bond b are each double bonds, and Q, Q₁, Q₂ and Q₃ are each CR_{2′}, for example, CH₂.

In some embodiments, Q₁, Q₂ and Q₃ are CH₂, k is 1, and Q is NR₁₃.

In some embodiments, n′ is 0. In some embodiments, n′ is 1. In other embodiments wherein n′ is 1, Y is CR₃R₄, for example, CH₂, X is OH, and L is CO₂H or an ester thereof.

In certain embodiments, n′ is 0, X is OH, and L is CO₂H or an ester thereof.

In some embodiments, Z is C₆₋₁₄ aryl, 5 to 13 membered heteroaryl, or 3 to 14 membered heterocyclo, each of which can be substituted with up to 3 substituents independently selected from halogen, C₁₋₁₀ alkyl, OC₁₋₁₀ alkyl, NO₂, NH₂, CN, CF₃, and CO₂H, CHO, CO₂H, C(═O)R₂₀, SO₂R₂₀, OH, C₁₋₆ alkyl, OC₁₋₆ alkyl, phenyl, benzyl, Ophenyl, Obenzyl, SO₂NH₂, SO₂NH(C₁₋₆ alkyl), SO₂N(C₁₋₆ alkyl)₂, CH₂COOH, CO₂Me, CO₂Et, CO₂iPr, C(═O)NH₂, C(═O)NH(C₁₋₆ alkyl), C(═O)N(C₁₋₆ alkyl)₂, and SC₁₋₆ alkyl.

In some embodiments, Z is selected from:
(a) a five-membered heterocyclic ring containing one to three ring heteroatoms selected from N, S or O; wherein said five-membered heterocyclic ring is optionally substituted by from 1 to 3 substituents selected from halogen, C₁₋₁₀ alkyl, OC₁₀ alkyl, NO₂, NH₂, CN, CF₃, and CO₂H;
(b) a six-membered heterocyclic ring containing one to three ring heteroatoms selected from N, S or O; wherein said six-membered heterocyclic ring is optionally substituted by from 1 to 3 substituents selected from halogen, C₁₀ alkyl, OC₁₋₁₀ alkyl, CHO, CO₂H, C(═O)R₂₀, SO₂R₂₀, NO₂, NH₂, CN, CF₃ and OH;
(c) a bicyclic ring moiety having 8 to 14 ring members, and optionally containing from 1 to 3 ring heteroatoms selected from N or O; wherein said bicyclic ring moiety is optionally substituted by from 1 to 3 substituents selected from halogen, C₁₋₆ alkyl, OC₁₋₆ alkyl, CHO, NO₂, NH₂, CN, CF₃, CO₂H, C(═O)R₂₀, SO₂R₂₀, and OH; and
(d) a benzyl, naphthyl, or phenyl ring, each of which is optionally substituted by from 1 to 3 substituents selected from halogen, C₁₋₆ alkyl, phenyl, benzyl, Ophenyl, Obenzyl, SO₂NH₂, SO₂NH(C₁₋₆ alkyl), SO₂N(C₁₋₆ alkyl)₂, CH₂COOH, CO₂H, CO₂Me, CO₂Et, CO₂iPr, C(═O)NH₂, C(═O)NH(C₁₋₆ alkyl), C(═O)N(C₁₋₆ alkyl)₂, OH, SC₁₋₆ alkyl, C₁₋₆ alkyl, NO₂, NH₂, CF₃, and CN.

In certain embodiments, R₁ and each R_{2′} are independently hydrogen, C₁₋₆alkyl, C₁₋₆ perhaloalkyl, OC₁₋₆alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, OSO₃R₆, SO₃R₆, PO₃R₆R₇, (CH₂)ₙSO₂NR₈R₉, (CH₂)ₙC(═O)NR₈R₉, NR₈R₉, C₆₋₁₄aryl, 3 to 14 membered heterocyclo, C(═O)R₁₂, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C(═O)C₇₋₂₄arylalkyl, OC(═O)C₇₋₂₄arylalkyl, OC₇₋₂₄arylalkyl, C₂₋₂₀ alkenyl, C₂₋₂₀ alkynyl, or NHCOR₈.

In some embodiments, Z is phenyl or a substituted phenyl (e.g., where the substituents are as described herein for aryl). In various embodiments, the methods of the present teachings comprise administering to a patient a compound having the Formula IV:

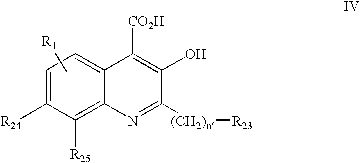

wherein:
n′ is 0 or 1;
R₁ is hydrogen, halogen, OH, CN, SH, C₁₋₆ alkyl, OC₁₋₆ alkyl, C₁₋₆ perhaloalkyl, C₁₋₆ thioalkyl, C₆₋₁₄ aryl or 5 to 13 membered heteroaryl; wherein said C₆₋₁₄ aryl and said 5 to 13 membered heteroaryl can each optionally be substituted with up to three substituents independently selected from halogen, OH, CN, SH, NH₂, C₁₋₆ alkyl, OC₁₋₆ alkyl, C₁₋₆ perhaloalkyl and C₁₋₆ thioalkyl; and
wherein said C₁₋₆ alkyl, OC₁₋₆ alkyl and C₁₋₆ thioalkyl can each optionally be substituted with up to three substituents independently selected from halogen, OH, CN, SH, NH₂, OC₁₋₆ alkyl, C₁₋₆ perhaloalkyl and C₁₋₆ thioalkyl;
R₂₃ is C₆₋₁₄ aryl or 5 to 13 membered heteroaryl, wherein said C₆₋₁₄aryl and 5 to 13 membered heteroaryl can each optionally be substituted with up to three substituents independently selected from halogen, OH, CN, SH, NH₂, C₁₋₆ alkyl, OC₁₋₆ alkyl, C₁₋₆ perhaloalkyl and C16 thioalkyl; and
R₂₄ and R₂₅ together form —(CH₂)₃—, —(CH₂)₄—, —(CH₂)₂—NH—, —(CH₂)₂—NH—CH₂— or —CH═CH—CH═CH—, wherein up to three hydrogens on the same or different atom(s) may independently be replaced with halogen, OH, CN, SH, NH₂, OC₁₋₆ alkyl, C16 perhaloalkyl, C(═O)R₂₀, SO₂R₂₀ and C16 thioalkyl; or
a pharmaceutically acceptable salt, hydrate or ester thereof.

In some embodiments, R₂₃ can be an optionally substituted C₆₋₁₄ aryl, for example, an optionally substituted phenyl. The phenyl can be substituted at the 4-position thereof, for example, by a substituent selected from halogen (e.g., Cl), OH, CN, SH, NH₂, CH₃, OCH₃, CF₃ and OCF₃.

In some embodiments, R₂₄ and R₂₅ together form an unsubstituted —(CH₂)₃—, —(CH₂)₄—, —(CH₂)₂—NH—, —(CH₂)₂—NH—CH₂— or —CH═CH—CH═CH—.

In some embodiments, R₁ is H, and R₂₄ and R₂₅ together form an unsubstituted —(CH₂)₃—. In yet other embodiments, R₁ is H, and R₂₄ and R₂₅ together form an unsubstituted —(CH₂)₄—. In yet another embodiment, R₁ is H, and R₂₄ and R₂₅ together form an unsubstituted —(CH₂)₂—NH—. In still other embodiments, R₁ is H, and R₂₄ and R₂₅ together form an unsubstituted —CH═CH—CH═CH—. In yet still other embodiments, R₁ is H, and R₂₄ and R₂₅ together form an optionally substituted —(CH₂)₂—NH—CH₂—.

In some embodiments, the present teachings provide a method for treating scleritis, a symptom of scleritis, or a scleritis-related disorder comprising administering to a subject a therapeutically effective amount of a compound selected from 2-(4-chloro-phenyl)-3-hydroxy-benzo[h]quinoline-4-carboxylic acid; 2-(4-chloro-phenyl)-3-hydroxy-7,8,9,10-tetrahydro-benzo[h]quinoline-4-carboxylic acid; 3-hydroxy-2-(4-trifluoromethoxy-benzyl)-7,8,9,10-tetrahydro-benzo[h]quinoline-4-carboxylic acid; 8-(4-chloro-benzyl)-7-hydroxy-2,3-dihydro-1H-aza-cyclopenta[a]naphthalene-6-carboxylic acid; 8-(4-chloro-benzyl)-7-hydroxy-2,3-dihydro-1H-pyrrolo[3,2-h]quinoline-6-carboxylic acid; f) 2-(4-chloro-benzyl)-3-hydroxy-7,8,9,10-tetrahydro-benzo[h]quinoline-4-carboxylic acid; triethylammonium 7,8-benzo-2-(4-chlorophenyl)-3-hydroxyquinoline-4-carboxylate; 2-(3,4-dichlorobenzyl)-3-hydroxy-7,8,9,10-tetrahydrobenzo[h]quinoline-4-carboxylic acid; 3-hydroxy-2-(thiophen-2-ylmethyl)-7,8,9,10-tetrahydrobenzo[h]quinoline-4-carboxylic acid; 2-(benzo[b]thiophen-3-ylmethyl)-3-hydroxy-7,8,9,10-tetrahydrobenzo[h]quinoline-4-carboxylic acid; 2-(2-chlorobenzyl)-3-hydroxy-7,8,9,10-tetrahydrobenzo[h]quinoline-4-carboxylic acid; 2-(3-chlorobenzyl)-3-hydroxy-7,8,9,10-tetrahydrobenzo[h]quinoline-4-carboxylic acid; 3-hydroxy-2-[2-(3-methylbenzo[b]thiophen-2-ylmethyl)]-7,8,9,10-tetrahydrobenzo[h]quinoline-4-carboxylic acid; 3-hydroxy-2-(thiophen-3-ylmethyl)-7,8,9,10-tetrahydrobenzo[h]quinoline-4-carboxylic acid; 3-hydroxy-2-(indol-3-ylmethyl)-7,8,9,10-tetrahydrobenzo[h]quinoline-4-carboxylic acid; 2-(5-chlorobenzo[b]thiophen-3-ylmethyl)-3-hydroxy-7,8,9,10-tetrahydrobenzo[h]quinoline-4-carboxylic acid; 3-hydroxy-2-phenyl-7,8,9,10-tetrahydro-benzo[h]quinoline-4-carboxylic acid; 2-(4-cyano-benzyl)-3-hydroxy-7,8,9,10-tetrahydro-benzo[h]quinoline-4-carboxylic acid; 2-(4-carboxy-benzyl)-3-hydroxy-7,8,9,10-tetrahydro-benzo[h]quinoline-4-carboxylic acid; 2-(4-carbamoyl-benzyl)-3-hydroxy-7,8,9,10-tetrahydro-benzo[h]quinoline-4-carboxylic acid; 2-benzyl-3-hydroxy-7,8,9,10-tetrahydro-benzo[h]quinoline-4-carboxylic acid; 3-hydroxy-2-phenethyl-7,8,9,10-tetrahydro-benzo[h]quinoline-4-carboxylic acid; 2-(4-chloro-benzyl)-3-hydroxy-7,8,9,10-tetrahydro-[1,9]phenanthroline-4-carboxylic acid; 2-(4-chloro-benzyl)-3-hydroxy-9-isopropyl-7,8,9,10-tetrahydro-[1,9]phenanthroline-4-carboxylic acid; 9-benzyl-2-(4-chloro-benzyl)-3-hydroxy-7,8,9,10-tetrahydro-[1,9]phenanthroline-4-carboxylic acid; 2-(4-chloro-benzyl)-9-ethyl-3-hydroxy-7,8,9,10-tetrahydro-[1,9]phenanthroline-4-carboxylic acid; 9-acetyl-2-(4-chloro-benzyl)-3-hydroxy-7,8,9,10-tetrahydro-[1,9]phenanthroline-4-carboxylic acid; 9-carbamoyl-2-(4-chloro-benzyl)-3-hydroxy-7,8,9,10-tetrahydro-[1,9]phenanthroline-4-carboxylic acid; 9-benzoyl-2-(4-chloro-benzyl)-3-hydroxy-7,8,9,10-tetrahydro-[1,9]phenanthroline-4-carboxylic acid; 9-benzoyl-3-benzoyloxy-2-(4-chloro-benzyl)-7,8,9,10-tetrahydro-[1,9]phenanthroline-4-carboxylic acid; 2-(4-chloro-benzyl)-3-hydroxy-9-methanesulfonyl-7,8,9,10-tetrahydro-[1,9]phenanthroline-4-carboxylic acid; 2-(4-chloro-benzyl)-3-hydroxy-7,10-dihydro-8H-[1,9]phenanthroline-4,9-dicarboxylic acid 9-ethyl ester; 2-(4-chloro-benzyl)-3-ethoxycarbonyloxy-7,10-dihydro-8H-[1,9]phenanthroline-4,9-dicarboxylic acid 9-ethyl ester; 2-(4-chloro-benzyl)-3-hydroxy-9-phenylacetyl-7,8,9,10-tetrahydro-[1,9]phenanthroline-4-carboxylic acid; 2-(4-chloro-benzyl)-3-hydroxy-9-(propane-2-sulfonyl)-7,8,9,10-tetrahydro-[1,9]phenanthroline-4-carboxylic acid; 2-(4-chloro-benzyl)-3-methoxy-7,8,9,10-tetrahydro-benzo[h]quinoline-4-carboxylic acid; 3-hydroxy-2-piperidin-4-yl-7,8,9,10-tetrahydro-benzo[h]quinoline-4-carboxylic acid; and 2-(1-acetyl-piperidin-4-yl)-3-hydroxy-7,8,9,10-tetrahydro-benzo[h]quinoline-4-carboxylic acid.

In some embodiments, substituent L is CO₂H, an ester thereof, or a pharmaceutically acceptable acid mimetic. As used herein, the term “acid mimetic” is intended to include moieties that mimic acid functionality in biological molecules. Examples of such acid mimetics are known in the art (see, e.g., R. B. Silverman, The Organic Chemistry of Drug Design and Drug Action, Academic Press Inc. (1992)), and include without limitation —OH and those shown below:

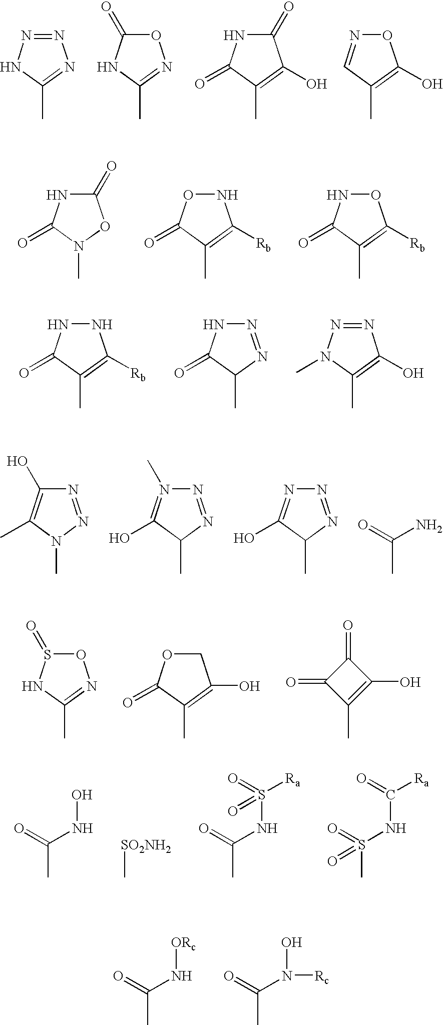

wherein:
Rₐ is selected from —CF₃, CH₃, phenyl and benzyl, wherein the phenyl or benzyl is optionally substituted with up to three groups independently selected from C₁₋₆ alkyl, C₁₋₆ alkoxy, C₁₋₆ thioalkyl, —CF₃, halogen, —OH and COOH;
R^{b} is selected from —CF₃, —CH₃, —NH₂, phenyl and benzyl, wherein the phenyl or benzyl is optionally substituted with up to three groups independently selected from C₁₋₆ alkyl, C₁₋₆ alkoxy, C₁₋₆ thioalkyl, —CF₃, halogen, —OH and COOH; and
R_{c} is selected from —CF₃ and C₁₋₆ alkyl.

Ester forms of the present compounds (e.g., compounds where L is an ester of CO₂H) include the pharmaceutically acceptable ester forms known in the art including those which can be metabolized into a free acid form, such as a free carboxylic acid, in the animal body, such as the corresponding alkyl esters (e.g., alkyl of 1 to 10 carbon atoms), cyclic alkyl esters, (e.g., of 3-10 carbon atoms), aryl esters (e.g., of 6-20 carbon atoms) and heterocyclic analogues thereof (e.g., of 3-20 ring atoms, 1-3 of which can be selected from oxygen, nitrogen and sulfur heteroatoms) can be used according to the present teachings. The alcohol residue can carry further substituents. Examples of esters include C₁-C₈ alkyl esters, for example, C₁-C₆ alkyl esters, such as methyl ester, ethyl ester, propyl ester, isopropyl ester, butyl ester, isobutyl ester, t-butyl ester, pentyl ester, isopentyl ester, neopentyl ester, hexyl ester; C₃₋₈ cyclic alkyl esters, for example, C₁₋₆ cyclic alkyl esters, such as cyclopropyl ester, cyclopropylmethyl ester, cyclobutyl ester, cyclopentyl ester, and cyclohexyl ester; and aryl esters such as phenyl ester, benzyl ester and tolyl ester.

As used herein, “halo” or “halogen” refers to fluoro (F), chloro (Cl), bromo (Br), and iodo (I).

As used herein, “oxo” refers to a double-bonded oxygen (i.e., ═O).

As used herein, the term “alkyl” as a group or part of a group is intended to denote hydrocarbon groups including straight chain, branched and cyclic saturated hydrocarbons. An alkyl group can contain 1-20 carbon atoms. A lower alkyl group can contain up to 4 carbon atoms or up to 6 carbon atoms. A cyclic alkyl group can be monocyclic (e.g., cyclohexyl) or polycyclic (e.g., containing fused, bridged, and/or spiro ring systems), wherein the carbon atoms can be located inside or outside of the ring system. Any suitable ring position of a cyclic alkyl group can be covalently linked to the defined chemical structure. Examples of straight chain and branched alkyl groups include methyl (Me), ethyl (Et), propyl (e.g., n-propyl and isopropyl), butyl (e.g., n-butyl, isobutyl, s-butyl, and t-butyl), pentyl groups (e.g., n-pentyl, isopentyl, and neopentyl), hexyl groups, and the like. Examples of cyclic alkyl groups include cyclopropyl, cyclobutyl, cyclopropylmethyl, cyclopentyl, cyclopentylmethyl, cyclohexyl, cyclohexylmethyl, cyclohexylethyl, and cycloheptyl. Unless otherwise indicated, alkyl groups are unsubstituted. However, where indicted, alkyl groups may be substituted with one or more independently selected substituents as described herein.

Throughout this specification, it should be understood that the term alkyl is intended to encompass both non-cyclic saturated hydrocarbon groups and cyclic saturated hydrocarbon groups. In some embodiments, alkyl groups are non-cyclic. In other embodiments, alkyl groups are cyclic. In various embodiments, alkyl groups are both cyclic and non-cyclic.

An alkyl group can include one or more halogen substituents, in which case the resulting group can be referred to as a “haloalkyl.” Examples of haloalkyl groups include, but are not limited to, CF₃, C₂F₅, CHF₂, CH₂F, CCl₃, CHCl₂, CH₂C₁, C₂Cl₅, CH₂CF₃, CH₂CH₂CF₂CH₃, CH(CF₃)₂, (CH₂)₆—CF₂CCl₃, and the like. “Perhaloalkyl” groups, i.e., alkyl groups wherein all of the hydrogen atoms are replaced with halogen atoms (e.g., CF₃ and C₂F₅), are included within the definition of “haloalkyl” but are also considered an independent subclass of haloalkyls.

As used herein, the term “alkenyl” is intended to denote an alkyl group that contains at least one carbon-carbon double bond. An alkenyl group can contain 2-20 carbon atoms, but can have a smaller range such as 2-6 carbon atoms and includes cyclic groups. Examples of alkenyl groups include, but are not limited to, ethenyl, propenyl, butenyl, pentenyl, hexenyl, butadienyl, pentadienyl, hexadienyl, vinyl, allyl, 2-methyl-allyl, 4-but-3-enyl, 4-hex-5-enyl, 3-methyl-but-2-enyl, cyclohex-2-enyl, and the like. The one or more carbon-carbon double bonds can be internal (such as in 2-butene) or terminal (such as in 1-butene). Examples of cyclic alkenyl groups include, but are not limited to, cyclopentenyl, cyclohexenyl, cyclohexadienyl, cycloheptatrienyl, and the like. Alkenyl groups may be substituted with one or more independently selected substituents as described herein.

As used herein, the term “alkynyl” is intended to denote an alkyl group that contains at least one carbon-carbon triple bond. An alkynyl group can contain 2-20 carbon atoms, but can have a smaller range such as 2-6 carbon atoms. Examples of alkynyl groups include, but are not limited to, ethynyl, propynyl, butynyl, pentynyl, pent-2-yne, ethynyl-cyclohexyl, and the like. The one or more carbon-carbon triple bonds can be internal (such as in 2-butyne) or terminal (such as in 1-butyne). Alkynyl groups may be substituted with one or more independently selected substituents as described herein.

In some embodiments, alkyl, alkenyl, and alkynyl groups as defined above can be substituted with one or more (e.g., up to four) independently selected substituents. In certain embodiments, these groups are substituted with one, two, or three independently selected substituents. Examples of such substituents include, among others, alkoxy (i.e., O-alkyl, e.g., lower alkoxy, e.g., O—C₁₋₆ alkyl), mono-, di- or trihaloalkoxy (e.g., —O—CX₃ where X is halogen), —(CH₂)ₙNH₂, —(CH₂)ₙNHBoc, C₁₋₆ alkyl, C₁₋₆ perhaloalkyl, OC₁₋₆ alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, C₆₋₁₄aryl, 3 to 14 membered heterocyclo, C(═O C₆₋₁₄aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, and NHCOR₈. Other examples of such substituents include phenyl, benzyl, O-phenyl, O-benzyl, —SO₂NH₂, —SO₂NH(C₁₋₆ alkyl), SO₂N(C₁₋₆ alkyl)₂, CH₂COOH, CO₂H, CO₂Me, CO₂Et, CO₂iPr, C(═O)NH₂, C(═O)NH(C₁-C₆), C(═O)N(C₁-C₆)₂, SC₁₋₆ alkyl, OC₁₋₆ alkyl, NO₂, NH₂, CF₃, and OCF₃. Unless specifically indicated otherwise, it is intended that the foregoing substituents for alkyl, alkenyl, and alkynyl groups not be further substituted.

As used herein, the term “alkoxy” refers to an —O-alkyl group, wherein alkyl is as defined herein. Examples of alkoxy groups include, but are not limited to, methoxy, ethoxy, propoxy (e.g., n-propoxy and isopropoxy), t-butoxy groups, and the like.

As used herein, “thioalkyl” refers to an —S-alkyl group, wherein alkyl is as defined herein. Examples of thioalkyl groups include, but are not limited to, methylthio, ethylthio, propylthio (e.g., n-propylthio and isopropylthio), t-butylthio groups, and the like. The alkyl portion of the moiety may be substituted with one or more independently selected substituents as described herein. Unless specifically indicated otherwise, it is intended that such substituents not be further substituted.

As used herein, the term “carbocyclic ring” refers to a saturated, partially saturated or aromatic ring system in which the ring atoms are each carbon. Carbocyclic rings may be substituted with one or more independently selected substituents as described herein. Unless specifically indicated otherwise, it is intended that such substituents not be further substituted.

As used herein, the term “aryl” as a group or part of a group refers to an aromatic monocyclic hydrocarbon ring system or a polycyclic ring system (e.g., bicyclic or tricyclic), e.g., of 6-14 carbon atoms where at least one of the rings present in the ring system is an aromatic hydrocarbon ring and any other aromatic rings present in the ring system include only hydrocarbons. In some embodiments, a monocyclic aryl group can have from 6 to 14 carbon atoms and a polycyclic aryl group can have from 8 to 14 carbon atoms. Any suitable ring position of the aryl group can be covalently linked to the defined chemical structure. In some embodiments, an aryl group can have only aromatic carbocyclic rings e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cyclic alkyl or heterocycloalkyl rings. Examples of such aryl groups include, among others, benzo derivatives of cyclopentane (i.e., an indanyl group, which is a 5,6-bicyclic cyclic alkyl/aromatic ring system), cyclohexane (i.e., a tetrahydronaphthyl group, which is a 6,6-bicyclic cyclic alkyl/aromatic ring system), imidazoline (i.e., a benzimidazolinyl group, which is a 5,6-bicyclic heterocycloalkyl/aromatic ring system), and pyran (i.e., a chromenyl group, which is a 6,6-bicyclic heterocycloalkyl/aromatic ring system). Other examples of aryl groups include, but are not limited to, benzodioxanyl, benzodioxolyl, chromanyl, indolinyl groups, and the like. In some embodiments, an aryl group can be substituted with one or more (e.g., up to 4) independently selected substituents. In certain embodiments, an aryl group is substituted with one, two, or three independently selected substituents. Examples of such substituents include, among others, alkoxy (i.e., O-alkyl, e.g., O—C₁₋₆ alkyl), mono-, di- or trihaloalkoxy (e.g., —O—CX₃ where X is halogen, e.g., —CH₂F, —CHF₂, or —CF₃), —(CH₂)ₙNH₂, —(CH₂)ₙNHBoc, C₁₋₆ alkyl, C₁₋₆ perhaloalkyl, OC₁₋₆ alkyl, OC₁₋₆ perhaloalkyl, halogen, thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, C₆₋₁₄aryl, 3 to 14 membered heterocyclo, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, and NHCOR₈, wherein the constituent variables are defined herein. Other examples of such substituents can include phenyl, benzyl, O-phenyl, O-benzyl, —SO₂NH₂, —SO₂NH(C₁₋₆ alkyl), SO₂N(C₁₋₆ alkyl)₂, CH₂COOH, CO₂H, CO₂Me, CO₂Et, CO₂iPr, C(═O)NH₂, C(═O)NH(C₁-C₆), C(═O)N(C₁-C₆)₂, SC₁₋₆ alkyl, OC₁₋₆ alkyl, NO₂, NH₂, CF₃, and OCF₃. Unless specifically indicated otherwise, it is intended that the foregoing substituents for aryl groups not be further substituted.

As used herein, the term “arylalkyl” refers to a group of the formula -alkyl-aryl, wherein aryl and alkyl have the definitions above. In some embodiments, an aryl alkyl group can be substituted with one or more (e.g., up to 4) independently selected substituents located on either the aryl or alkyl portion of the moiety, or both the aryl and the alkyl portions of the moiety. In certain embodiments, an arylalkyl group is substituted with one, two, or three independently selected substituents. Examples of such substituents include, among others, alkoxy (i.e., O-alkyl, e.g., O—C₁₋₆ alkyl), mono-, di- or trihaloalkoxy (e.g., —O—CX₃ where X is halogen), —(CH₂)ₙNH₂, —(CH₂)ₙNHBoc, C₁₋₆ alkyl, C₁₋₆ perhaloalkyl, OC₁₋₆alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, C₆₋₁₄aryl, 3 to 14 membered heterocyclo, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄ aryl, 3 to 14 membered Oheterocyclo, C₇₋₂₄arylalkyl, C(═O)C₇₋₂₄arylalkyl, OC(═O)C₇₋₂₄arylalkyl, OC₇₋₂₄ arylalkyl, C₂₋₂₀ alkenyl, C₂₋₂₀ alkynyl, and NHCOR₈, wherein the constituent variables are defined herein. Other examples of such substituents include phenyl, benzyl, O-phenyl, O-benzyl, —SO₂NH₂, —SO₂NH(C₁₋₆ alkyl), SO₂N(C₁₋₆ alkyl)₂, CH₂COOH, CO₂H, CO₂Me, CO₂Et, CO₂₁Pr, C(═O)NH₂, C(═O)NH(C₁-C₆), C(═O)N(C₁-C₆)₂, SC₁₋₆ alkyl, OC₁₋₆ alkyl, NO₂, NH₂, CF₃, and OCF₃. In some embodiments, the arylalkyl group is a benzyl group that is optionally substituted with 1 to 3 independently selected substituents as described above. Unless specifically indicated otherwise, it is intended that the foregoing substituents for arylalkyl groups not be further substituted.

As used herein, “heteroatom” refers to an atom of any element other than carbon or hydrogen and includes, for example, nitrogen, oxygen, sulfur, phosphorus, and selenium.

As used herein, the term “heterocyclo” as a group or part of a group, refers to a mono-, bi-, or higher order cyclic ring system (e.g., of 3-14 ring atoms) that contains at least one ring heteroatom (e.g., oxygen, nitrogen or sulfur), and optionally contains one or more double or triple bonds. One or more N or S atoms in a heterocyclo can be oxidized (e.g., morpholine N-oxide, thiomorpholine S-oxide, thiomorpholine S,S-dioxide). Heterocyclo groups may be substituted with one or more independently selected substituents as described herein (e.g., as for aryl as described above). Unless specifically indicated otherwise, it is intended that such substituents not be further substituted. In some embodiments, nitrogen atoms of heterocycloalkyl groups can bear a substituent as described herein. Heterocyclo groups can include fully saturated and partially saturated cyclic heteroatom-containing moieties (containing, e.g., none, or one or more double bonds). Such fully and partially saturated cyclic non-aromatic groups are also collectively referred to herein as “heterocycloalkyl” groups. Heterocycloalkyl groups can also contain one or more oxo groups, such as phthalimide, piperidone, oxazolidinone, pyrimidine-2,4(1H,3H)-dione, pyridin-2(1H)-one, and the like. Examples of heterocycloalkyl groups include, among others, morpholine, thiomorpholine, pyran, imidazolidine, imidazoline, oxazolidine, pyrazolidine, pyrazoline, pyrrolidine, pyrroline, tetrahydrofuran, tetrahydrothiophene, piperidine, piperazine, and the like.

Heterocyclo groups also include cyclic heteroatom-containing moieties that contain at least one aromatic ring. Such fully and partially aromatic moieties are also collectively referred to herein as “heteroaryl” groups. A heteroaryl group, as a whole, can have, for example, from 5 to 13 ring atoms and contain 1-5 ring heteroatoms. Heteroaryl groups can include monocyclic heteroaryl rings fused to one or more aromatic carbocyclic rings, non-aromatic carbocyclic rings, and non-aromatic heterocycloalkyl rings. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered monocyclic and 5-6 bicyclic ring systems shown below:

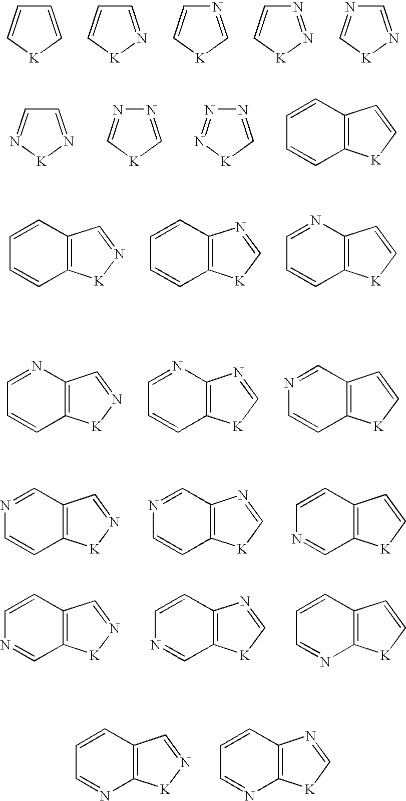

where K is O, S, NH, or NR₃, wherein R₃ is as defined herein or any other substituent that is suitable for a tertiary nitrogen ring atom. Examples of such heteroaryl rings include, but are not limited to, pyrrole, furan, thiophene, pyridine, pyrimidine, pyridazine, pyrazine, triazole, tetrazole, pyrazole, imidazole, isothiazole, thiazole, thiadiazole, isoxazole, oxazole, oxadiazole, indole, isoindole, benzofuran, benzothiophene, quinoline, 2-methylquinoline, isoquinoline, quinoxaline, quinazoline, benzotriazole, benzimidazole, benzothiazole, benzisothiazole, benzisoxazole, benzoxadiazole, benzoxazole, cinnoline, 1H-indazole, 2H-indazole, indolizine, isobenzofuran, naphthyridine, phthalazine, pteridine, purine, oxazolopyridine, thiazolopyridine, imidazopyridine, furopyridine, thienopyridine, pyridopyrimidine, pyridopyrazine, pyridopyridazine, thienothiazole, thienoxazole, and thienoimidazole. Further examples of heteroaryl groups include, but are not limited to, 4,5,6,7-tetrahydroindole, tetrahydroquinoline, benzothienopyridine, benzofuropyridine, and the like. In other embodiments, heteroaryl groups can be substituted with one or more (e.g., up to four) independently selected substituents as described herein (e.g., as for aryl as described above). Unless specifically indicated otherwise, it is intended that such substituents not be further substituted.

In other embodiments, heterocyclo groups can be:
(a) five-member heterocyclic rings containing one to three ring heteroatoms selected from N, S and O such as, but not limited to, furan, imidazole, imidazolidine, isothiazole, isoxazole, oxathiazole, oxazole, oxazoline, pyrazole, pyrazolidine, pyrazoline, pyrrole, pyrrolidine, pyrroline, thiazoline, or thiophene, the five-member heterocyclic ring being optionally substituted with from 1 to 3 substituents independently selected from halogen and C₁₋₁₀ alkyl, for example, C₁₋₆ alkyl, OC₁₋₁₀ alkyl, OC₁₋₆ alkyl, NO₂, NH₂, CN, CF₃, and CO₂H; or
(b) six-member heterocyclic rings containing one to three ring heteroatoms selected from N, S and O such as, but not limited to morpholine, oxazine, piperazine, piperidine, pyran, pyrazine, pyridazine, pyridine, pyrimidine, thiadizine, or thiazine, the six-member heterocyclic ring being optionally substituted with from 1 to 3 substituents independently selected from halogen, C₁₋₁₀ alkyl, OC₁₋₁₀ alkyl, CHO, CO₂H, C(═O)R₂₀, SO₂R₂₀, NO₂, NH₂, CN, CF₃ and OH; or
(c) a bicyclic ring moiety having 8 to 14 ring members, and optionally containing from 1 to 3 ring heteroatoms selected from N and O such as, but not limited to, benzodioxine, benzodioxole, benzofuran, chromene, cinnoline, indazole, indole, indoline, indolizine, isoindole, isoindoline, isoquinoline, naphthalene, naphthyridine, phthalazine, purine, quinazoline, quinoline, or quinolizine, the bicyclic ring moiety being optionally substituted with from 1 to 3 substituents independently selected from halogen, C₁₋₆ alkyl, OC₁₋₆ alkyl, CHO, NO₂, NH₂, CN, CF₃, CO₂H, C(═O)R₂₀, SO₂R₂₀, and OH.

As used herein, the term “x-y membered” when used in conjunction with a group having one or more rings, is intended to mean that the group has the number of ring atoms indicated by integers “x” and “y”. For example, the term “3-14 membered heterocyclo” is intended to mean a heterocyclo group having 3-14 ring atoms. Similarly, the term “3-14 membered —OC(═O)heterocyclo” means a group of formula —OC(═O)-heterocyclo where the heterocyclo portion thereof contains 3-14 ring atoms.

At various places in the present specification, substituents of compounds are disclosed in groups or in ranges. It is intended that the description include each and every individual sub combination of the members of such groups and ranges. For example, the term “C₁₋₁₀ alkyl” is intended to disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of another example, the term “5-13 member heteroaryl group” is intended to individually disclose a heteroaryl group having 5, 6, 7, 8, 9, 10, 11, 12, 13, 5-13, 5-12, 5-11, 5-10, 5-9, 5-8, 5-7, 5-6, 6-13, 6-12, 6-11, 6-10, 6-9, 6-8, 6-7, 7-13, 7-12, 7-11, 7-10, 7-9, 7-8, 8-13, 8-12, 8-11, 8-10, 8-9, 9-13, 9-12, 9-11, 9-10, 10-13, 10-12, 10-11, 11-13, 11-12, and 12-13 ring atoms.

Compounds described herein can contain an asymmetric atom (also referred to as a chiral center), and some of the compounds can contain one or more asymmetric atoms or centers, which can thus give rise to optical isomers (enantiomers) and diastereomers. The present teachings and compounds disclosed herein include such optical isomers (enantiomers) and diastereomers (geometric isomers), as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

It is contemplated that the present teachings also include all possible protonated and unprotonated forms of the compounds described herein, as well as solvates, tautomers and pharmaceutically acceptable salts thereof.

Throughout the specification, structures may or may not be presented with chemical names. Where any question arises as to nomenclature, the structure prevails.

The compounds of the present teachings can be useful for the treatment or inhibition of a pathological condition or disorder, for example, scleritis or a scleritis-related disorder, in a mammal, for example, a human. The present teachings accordingly include a method of providing to a mammal a pharmaceutical composition that comprises a compound of the present teachings in combination or association with a pharmaceutically acceptable carrier. The compound of the present teachings can be administered alone or in combination with other therapeutically effective compounds or therapies for the treatment or inhibition of the pathological condition or disorder, for example, scieritis or a scleritis-related disorder.

Additionally, the compounds of the present teachings can be useful for the reduction and/or prevention of leukocyte adhesion to the vascular endothelium.

The present teachings include use of the compounds disclosed herein as active therapeutic substances for the treatment or inhibition of a pathological condition or disorder, for example, scleritis or a scleritis-related disorder. Accordingly, the present teachings further provide methods of treating these pathological conditions or disorders using the compounds described herein. In some embodiments, the methods include identifying a mammal having a pathological condition or disorder characterized by a scleritis symptom or scleritis-related symptom and providing to the mammal in need thereof an effective amount of a compound as described herein.

Pharmaceutically acceptable salts of the compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal and alkaline earth metal salts, for example, sodium, potassium, magnesium salts; ammonia salts; and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, naphthalenesulfonic, nitric, oxalic, pamoic, pantothenic, phosphoric, phthalic, propionic, succinic, sulfuric, tartaric, toluenesulfonic, and any other known pharmaceutically acceptable acids.

The compounds described herein can also be administered in the form of liposomes. As is known in the art, liposomes are generally derived from phospholipids or other lipid substances, and are formed by mono or multilamellar hydrated liquid crystals that are dispersed in an aqueous medium. Any nontoxic, pharmacologically acceptable lipid capable of forming liposomes can be used.

The present teachings also include prodrugs of the compounds described herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds as described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include, but are not limited to, acetate, formate and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

The present teachings provide pharmaceutical compositions comprising at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. Pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders, tablet-disintegrating agents, or encapsulating materials. The compounds can be formulated in a conventional manner, for example, in a manner similar to that used for known anti-inflammatory agents. Oral formulations containing an active compound disclosed herein can comprise any conventionally used oral form, including tablets, capsules, buccal forms, troches, lozenges, oral liquids, suspensions and solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets may contain up to 99% of the active compound.

Capsules can contain mixtures of active compound(s) with inert filler(s) and/or diluent(s) such as the pharmaceutically acceptable starches (e.g., corn, potato and tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending and stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also consist of administering an active compound in water and/or fruit juice, containing appropriate solubilizers and/or emulsifiers as needed.

Liquid carriers can be used in preparing solutions, suspensions, emulsions, syrups, and elixirs. An active compound described herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

The pharmaceutical composition can be in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts thereof, in lotions, creams, foams, patches, suspensions, solutions, and suppositories (rectal and vaginal).

When administered for the treatment or inhibition of a particular disease state or disorder, e.g., scleritis or a scleritis-related disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from the disease in an amount sufficient to cure or at least partially ameliorate the symptoms of the disease and its complications. An amount adequate to accomplish this result is defined as a “therapeutically effective amount.” The dosage to be used in the treatment of a specific individual typically must be subjectively determined by the attending physician. The variables involved include the specific condition and its state as well as the size, age and response pattern of the patient.

In some cases, it may be desirable to administer a compound directly to the airways of the patient in the form of an aerosol. For administration by intranasal or intrabronchial inhalation, the compounds of the present teachings can be formulated into an aqueous or partially aqueous solution.

Compounds described herein can be administered parenterally or intraperitoneally. Solutions or suspensions of these active compounds or pharmaceutically acceptable salts thereof can be prepared in water suitably mixed with a surfactant such as, for example, hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions and dispersions. For example, in certain embodiments, the form is sterile and its viscosity permits it to flow through a syringe. The form should be stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally (i.e., administered across the surface of the body and the inner linings of bodily passages including epithelial and mucosal tissues). Such administration can be carried out using the compounds of the present teachings including pharmaceutically acceptable salts thereof, in lotions, creams, foams, patches, suspensions, solutions, and suppositories (rectal and vaginal). Topical formulations that deliver active compound(s) through the epidermis can be useful for localized treatment of inflammation and arthritis.

Transdermal administration can be accomplished through the use of a transdermal patch containing an active compound and a carrier that can be inert to the active compound, non-toxic to the skin, and allow delivery of the active compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound are also suitable. A variety of occlusive devices can be used to release the active compound into the blood stream, such as a semi-permeable membrane covering a reservoir containing the active compound with or without a carrier, or a matrix containing the active compound. Other occlusive devices and methods are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as, for example, polyethylene glycols of various molecular weights, can also be used.

Lipid formulations or nanocapsules can be used to introduce compounds of the present teachings into host cells either in vitro or in vivo. Examples of lipids include, among others, natural and synthetic phospholipids, e.g., phosphatidyl cholines (lecithins). Lipid formulations and nanocapsules can be prepared by methods known in the art.

To increase the effectiveness of compounds of the present teachings, they can be combined with other agents effective in the treatment of the target disease. For inflammatory diseases, other active compounds (i.e., other active ingredients or agents) effective in their treatment, and particularly in the treatment of scleritis, can be administered with active compounds of the present teachings. Examples include NSAIDs (e.g. ibuprofen or indomethacin); immunomodulatory drugs; and steroidal compounds such as, for example, prednisone and cortisone-related drugs (e.g. corticosteroid). Such agents can be administered at the same time or at different times than the compounds disclosed herein.

Where an element or component is said herein to be included in and/or selected from a list of recited elements or components, it should be understood that the element or component can be any one of the recited elements or components and can be independently selected from a group consisting of two or more of the recited elements or components.

The use of the singular herein includes the plural (and vice versa) unless specifically stated otherwise. In addition, where the use of the term “about” is used in conjunction with a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

The compounds of the present teachings can be prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or specific process conditions (e.g. reaction temperatures, times, mole ratios of reactants, solvents, and pressures) are given, other process conditions can also be used unless otherwise stated. Optimum reaction conditions may vary with the particular reactants or solvent used, but one skilled in the art can determine such conditions by routine optimization procedures. Those skilled in the art of organic synthesis will recognize that the nature and order of the synthetic steps presented may be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., 1H or 13C), infrared spectroscopy, spectrophotometry (e.g., UV-visible), or mass spectrometry, or by chromatography such as high performance liquid chromatography (HPLC) or thin layer chromatography.

Preparation of compounds can involve the protection and deprotection of various chemical groups. One skilled in the art can readily determine the need for protection and deprotection and the selection of appropriate protecting groups. The chemistry of protecting groups can be found, for example, in Greene, et al., Protective Groups in Organic Synthesis, 2d. Ed., Wiley & Sons, 1991, the entire disclosure of which is incorporated by reference herein for all purposes.

The reactions of the processes described herein can be carried out in suitable solvents, which can be readily selected by one skilled in the art of organic synthesis. Suitable solvents typically are substantially nonreactive with the reactants, intermediates, and/or products at the temperatures at which the reactions are carried out (i.e. temperatures that can range from the solvent's freezing temperature to the solvent's boiling temperature). A given reaction can be carried out in one solvent or a mixture of more than one solvent. Depending on the particular reaction step, suitable solvents for a particular reaction step can be selected by one of skill in the art.

### EXAMPLES

The following examples illustrate various synthetic routes that can be used to prepare compounds of the present teachings.

The compounds of the present teachings can be readily prepared according to a variety of synthetic manipulations, all of which would be familiar to one skilled in the art.

A representative general synthetic scheme for the preparation of compounds of the present teachings is set forth below.

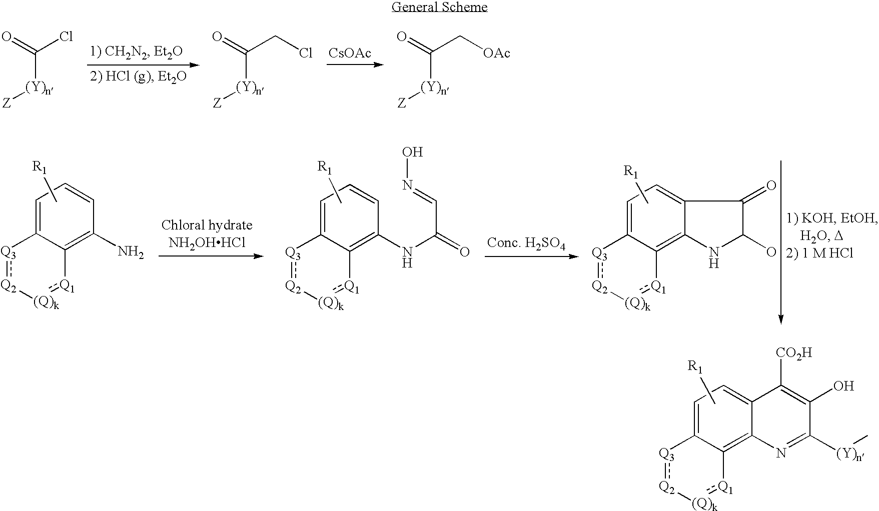

### General Scheme

Those of skill in the art will appreciate that a wide variety of compounds of the present teachings can be prepared according to the General Scheme above. For example, by starting with an appropriately substituted phenacetyl chloride, numerous differently substituted benzyl groups at the quinoline 2-position can be prepared. Likewise, one skilled in the art also recognizes that variously substituted anilines can be purchased or prepared and used for the construction of variously substituted quinoline rings as described in, for example, Formula I. Additionally, protection of the carboxylic acid via esterification (e.g., with an alcohol) or some other masking reaction can allow for selective alkylation, acylation, or other functionalization of the 3-hydroxy group located on the quinoline ring.

While the present teachings have been described with specificity in accordance with certain of their embodiments, the following examples serve only to illustrate the present teachings and are not intended to limit the same.

### Example 1

### Preparation of 2-(4-chloro-benzyl)-3-hydroxy-7,8,9,10-tetrahydro-benzo[h]quinoline-4-carboxylic acid (Compound 1)

### Compound 1 was Prepared According to Scheme 1 Below:

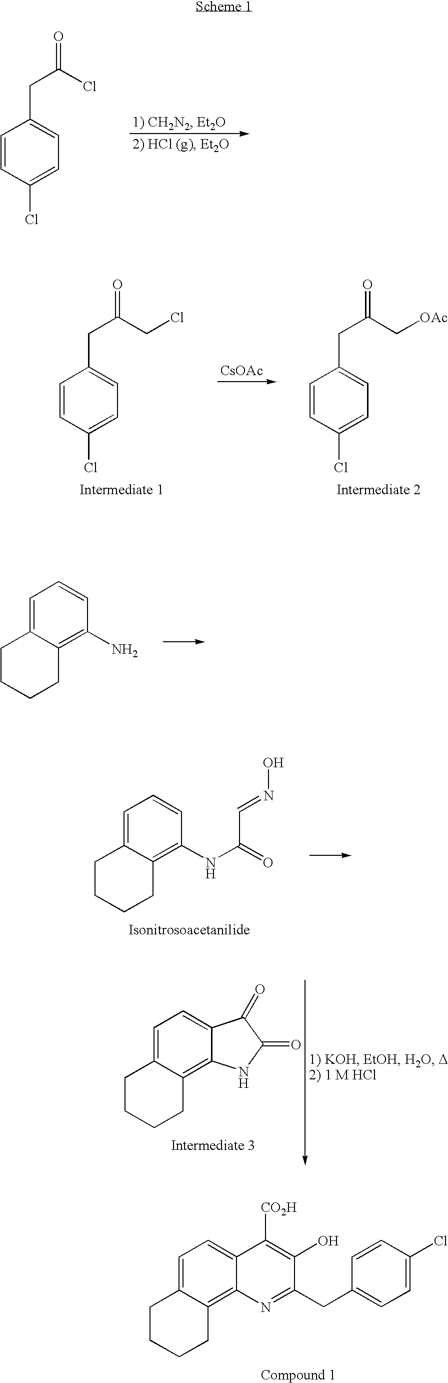

### Intermediate 1: 1-chloro-3-(4-chloro-phenyl)-propan-2-one

A solution of 30 g (158.7 mmol) of p-chlorophenacetyl chloride in 200 mL of ether was added over 30 min to 420 mL of diazomethane in ether (0.57 mmol/mL) white stirring in an ice bath, [Diazomethane was prepared using the procedure described in Org. Syn. Coll. Vol. II pages 165-167]. The reaction was stirred in ice for 3 h, then overnight at room temperature. Next, a gentle stream of anhydrous HCl gas was passed through the solution of the diazoketone at 0-4° C. for ca. 5-8 min, till the evolution of nitrogen ceased. After an additional hour in the ice bath, the reaction was poured into 700 mL crushed ice-water. The mixture was stirred 15 minutes diluted with 400 mL ether and the organic phase was washed with 750 mL of a 5% sodium carbonate solution, then 500 mL semi-saturated brine. The combined organic layers dried (sodium sulphate) ether solutions were evaporated to yield 25.5 g of crude intermediate 1 as a pale yellow solid. A solution of the crude was dissolved in 30-35 mL of methylene chloride was purified by flash chromatography on 500 g silica gel 60 (Merck 0.04-0.063 mm). Elution of the column (40×6 cm) with ethyl acetate-hexanes 20:80 gave 21.1 g (65.3% yield) of the pure intermediate 1 as colorless crystals. ¹H NMR (CDCl₃, 300 MHz), δ ppm 3.88 (s, 2H) 4.11 (s, 2H) 7.16 (d, J=8.59 Hz, 2H) 7.32 (d, J=8.59 Hz, 2H).

### Intermediate 2: acetic acid 3-(4-chloro-phenyl)-2-oxo-propyl ester

To a gently refluxing solution of 21.1 g (103.9 mmol) of Intermediate 1 in 200 mL ethanol was added in one portion 21.94 g (114.3 mmol, 1.1 equiv.) cesium acetate in 100 mL water and 10 mL glacial acetic acid. After refluxing for 3 hours the reaction reached an optimal stage (TLC: ethyl acetate:hexanes 20:80, ammonium molybdate spray). Most of the ethanol was removed by evaporation and the resulting oily mixture was distributed between 2×800 mL portions of ethyl acetate and 2×500 mL ice cold semi saturated sodium bicarbonate solution. The organic layers were washed in sequence with 500 mL brine, dried sodium sulfate, and evaporated in vacuo. A solution of the residue in 30 mL methylene chloride was purified by flash chromatography on 500 g silica gel. Elution of the column with ethyl acetate:hexanes 20:80 to 30:70 afforded 12.09 g (51.3%) of the intermediate 2 as a colorless crystalline solid. Recrystallization from ether:hexanes provided 11.7 g of pure intermediate 2. 1.88 g of starting material was also recovered. ¹H NMR (CDCl3, 300 MHz), δ ppm 2.16 (s, 3H) 3.72 (s, 2H) 4.69 (s, 2H) 7.15 (d, J=8.59 Hz, 2H) 7.31 (d, J=8.59 Hz, 2H).

### Intermediate 3: 6,7,8,9-tetrahydro-1H-benzo[g]indole-2,3-dione

Isatin synthesis described by Yang et al. (J. Am. Chem. Soc., 1996, 118, 9557) was used. Chloral hydrate (3.28 g, 19.8 mmol), hydroxylamine hydrochloride (4.13 g, 59.4 mmol) and sodium sulfate (23 g, 165 mmol) were placed in a 500 mL round-bottomed flask, and 120 mL water was added. The suspension was heated to 55° C. under a N₂ balloon until all the solids had dissolved, and an emulsion of 5,6,7,8-tetrahydro-naphthalen-1-ylamine (Aldrich, 2.43 g, 16.5 mmol) in 2 M aqueous hydrochloric acid was then added. Heating was continued overnight. After 18 hours, the reaction mixture was cooled to room temperature. The brown, lumpy precipitate was collected by filtration, washing with water, and dried overnight to give isoniirosoacetanilide (3.4 g). Isonitrosoacetanilide (3.4 g) was added in small portions, with stirring, to 12.4 mL concentrated sulfuric acid, which had been heated to 65° C. in a round bottom flask. The isonitroso was added slowly. After all the isonitroso had been added, the purplish-black solution was allowed to stir at 85° C. for 10 minutes, and was then poured onto crushed ice in a beaker. Additional ice was added until the outside of the beaker felt cold to the touch. The orange-brown precipitate was then collected by filtration and dried overnight to yield isatin 3, which was purified by extraction. Intermediate 3 (5.7 g) was extracted with 3×400 mL hot ethyl acetate and the insoluble was discarded. Evaporation of ethyl acetate gave 3.83 g of pure material. ¹H NMR (400 MHz, DMSO-D₆) δ ppm 1.74 (m, 4H) 2.50 (m, 2H) 2.74 (t, J=5.81 Hz, 2H) 6.79 (d, J=7.83 Hz, 1H) 7.23 (d, J=7.83 Hz, 1H) 10.95 (s, 1H).

### 2-(4-chloro-benzyl)-3-hydroxy-7,8,9,10-tetrahydro-benzo[h]quinoline-4-carboxylic acid (Compound 1)

Addition of 6.8 g (33.8 mmol) of isatin 3 to 60 mL of 6 N KOH at 100° C. afforded after stirring for 5 minutes a clear yellow brown solution of hydrolyzed isatin. To this was added in small portions while stirring at 100° C., a solution of 13.7 g (60.83 mmol, 1.8 equiv.) of the intermediate 2 in 120 mL lukewarm ethanol over a period of 1.5 hours. The clear solution was refluxed 1 h longer. After cooling to room temp., the reaction was diluted with 300 mL water under vigorous stirring then acidified by very slow addition of diluted HCl (1:4 conc. HCl:water) over 1.5 hours to pH<0. The reaction was stirred overnight and filtered. The crude material was purified by column chromatography eluting with ethyl acetate:acetonitrile:methanol:water 70:5:2.5:2.5+0.5% triethylamine followed by ethyl acetate:acetonitrile:methanol:water 70:10:5:5+0.5% triethylamine. The triethyl amine salt was converted to the free acid by dissolving the salt (0.625 g) in 500 mL ethyl acetate and 220 mL water containing 20 mL dilute HCl(1:5). The organic layer was washed with brine, dried (sodium sulfate) and concentrated to a small volume when the free acid just crashed out to give canary yellow crystals of pure Compound 1 (0.512 g). Total yield was 40.8%. ¹H NMR (400 MHz, DMSO-d₆) δ ppm 1.82 (m, 4H) 2.83 (t, J=5.56 Hz, 2H) 3.16 (t, J=5.68 Hz, 2H) 4.31 (s, 2H) 7.29 (d, J=8.84 Hz, 1H) 7.34 (s, 4H) 8.18 (d, J=8.84 Hz, 1H).

### Example 2

### Preparation of 2-(4-chlorophenyl)-3-hydroxy-7,8,9,10-tetrahydrobenzo[h]quinoline-4-carboxylic acid (Compound 2)

### Compound 2 was Prepared According to Scheme 2 Below:

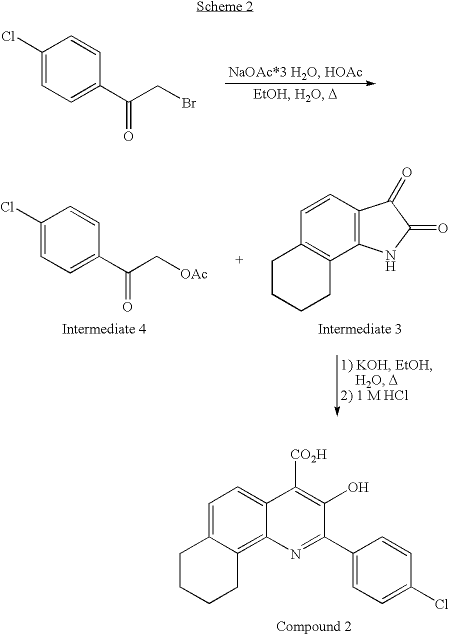

### Intermediate 4: 4-chlorophenacyl acetate

Compound 2 was prepared as described by Cragoe et al. (J. Org. Chem., 1953, 18, 561), except that the phenacyl bromide was used instead of the phenacyl chloride. A suspension of 2-bromo-4′-chloroacetophenone (Aldrich, 50 g, 0.21 mol) in 220 mL ethanol was prepared in a 1 L round-bottomed flask, and a solution of sodium acetate trihydrate (32 g, 0.24 mol) in 110 mL water and 11 mL acetic acid was added. The mixture was heated at reflux for 2.5 hours, then cooled to room temperature and refrigerated overnight. The white crystalline material which precipitated was collected by filtration, washing once with a cold solution of 50% aqueous ethanol, and dried under vacuum to give pure phenacyl acetate 4 (38 g, 83% yield): ¹H NMR (400 MHz, CDCl₃) δ ppm 2.22 (s, 3H) 5.28 (s, 2H) 7.46 (d, J=8.59 Hz, 2H) 7.85 (d, J=8.59 Hz, 2H).

### 2-(4-chlorophenyl)-3-hydroxy-7,8,9,10-tetrahydrobenzo[h]quinoline-4-carboxylic acid (Compound 2)

The procedure described by Cragoe et al. (J. Org. Chem., 1953, 18, 561) was followed. A suspension of Intermediate 3 (15.0 g, 74.3 mmol) in 80 mL 6 M aqueous potassium hydroxide was prepared in a 1 L 3-necked round-bottomed flask fitted with a reflux condenser, and heated to 100° C. A solution of Intermediate 4 (19.7 g, 92.9 mmol) in 80 mL warm ethanol was added in small portions over the course of 1 hour. After all this solution had been added, the reaction mixture was heated at reflux for an additional 4 hours. It was then cooled to room temperature, and the ethanol removed under reduced pressure. The residue was diluted with 385 mL water, chilled for 30 minutes, filtered, and acidified to pH 1 with 1 M aqueous hydrochloric acid. The crude acid precipitate was collected by filtration and dried under vacuum. To purify the acid, it was first eluted over a silica gel column (flash chromatography, 70 ethyl acetate:5 acetonitrile:2.5 methanol:2.5 water [+0.5% triethylamine]) to remove most of the highly colored impurities. The triethylammonium salt obtained was then suspended in 20% acetonitrile/water and converted back to the free acid by addition of concentrated hydrochloric acid. The acid precipitate was collected once again by filtration, dried under vacuum, and recrystallized in several batches from chloroform/ethanol to give pure Compound 2 as a pale yellow powder (3.03 g, 12% yield): ¹H NMR (400 MHz, DMSO-D₆) δ ppm 1.84 (m, 4H) 2.85 (t, J=5.56 Hz, 2H) 3.25 (t, J=5.56 Hz, 2H) 7.33 (d, J=8.84 Hz, 1H) 7.58 (d, J=8.59 Hz, 2H) 8.15 (d, J=8.59 Hz, 2H) 8.26 (d, J=8.84 Hz, 1H).

### Example 3

### Preparation of triethylammonium 7,8-benzo-2-(4-chlorophenyl)-3-hydroxyquinoline-4-carboxylate (Compound 3)

### Compound 3 was Prepared According to Scheme 3 Below:

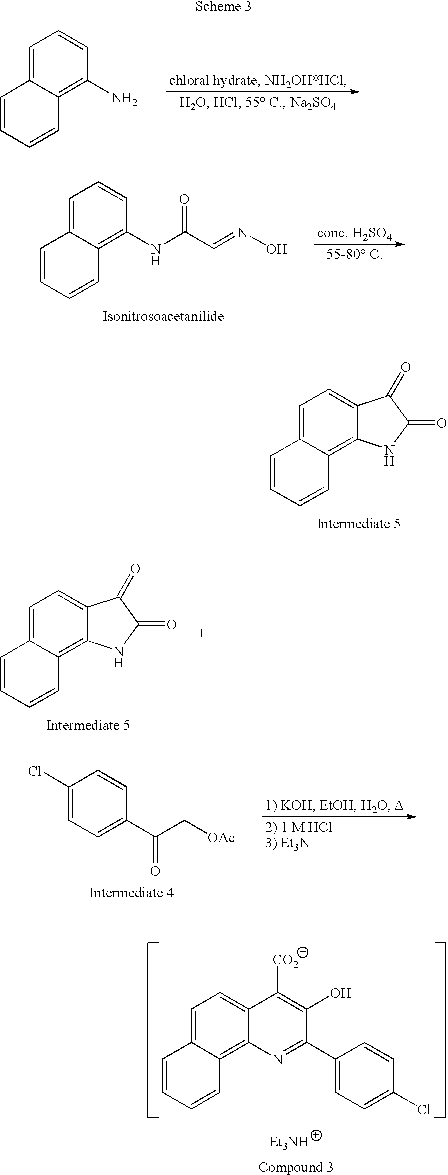

### Intermediate 5: 1H-benzo[g]indole-2,3-dione

The procedure described above for the synthesis of intermediate 3 was followed, reacting 1-aminonaphthalene (10.0 g, 69.8 mmol) with chloral hydrate (13.9 g, 83.8 mmol) and hydroxylamine hydrochloride (17.5 g, 0.251 mol) in the presence of sodium sulfate (99 g, 0.70 mol). Isonitrosoacetanilide was obtained as a brownish-black solid (7.09 g, 47% yield).

Cyclization was also carried out as described above. After pouring the reaction mixture onto ice and chilling it in the fridge overnight, a small amount of black precipitate had appeared. This was collected by filtration, washed with water (3×), and dried under vacuum. The filtrate was extracted into ethyl acetate as described to give blacker solid. Both samples contained some of the desired isatin 5, but were very impure (2.19 g, 34% yield).

### Triethylammonium 7,8-benzo-2-(4-chlorophenyl)-3-hydroxyquinoline-4-carboxylate (Compound 3)

The procedure described above for the synthesis of Compound 2 was followed, reacting intermediate 5 (2.19 g, 11.1 mmol) with 4-chlorophenacyl acetate (intermediate 4, 2.95 g, 13.9 mmol). The crude acid was purified by flash chromatography over silica gel (70 ethyl acetate:5 acetonitrile:2.5 methanol:2.5 water [+0.5% triethylamine]). The product was not pure enough and therefore purified again by Discovery Analytical Chemistry (preparative HPLC, acetonitrile/water/triethylamine). After lyophilization, product Compound 3 was obtained as the triethylammonium salt, a yellow solid (54 mg, 1.1% yield): ¹H NMR (400 MHz, DMSO-D₆) δ 1.17 (t, J=7.3 Hz, 9H) 3.09 (m, 6H) 7.57 (m, 3H) 7.65 (m, 1H) 7.80 (d, J=9.1 Hz, 1H) 7.89 (d, J=8.6 Hz, 1H) 8.55 (dt, J=9.1, 2.5, 2.3 Hz, 2H) 9.13 (d, J=8.8 Hz, 1H) 9.53 (d, J=9.4 Hz, 1H); HRMS (ESI+) calcd for C₂₀H₁₃ClNO₃ 350.0579, found 350.0580.

### Preparation of 8-(4-chlorobenzyl)-7-hydroxy-2,3-dihydro-1H-pyrrolo[3,2-h]quinoline-6-carboxylic acid (Compound 4)

### Compound 4 was Prepared According to Scheme 4 Below:

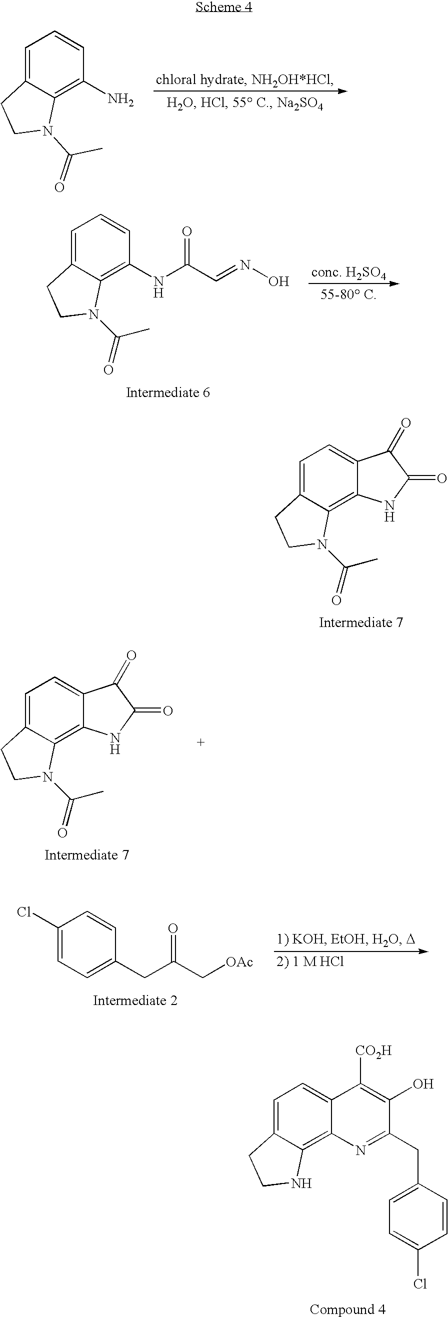

### Intermediate 6: N-(1-acetyl-2,3-dihydro-1H-indol-7-yl)-2-hydroxyimino-acetamide

Intermediate 6 was synthesized according to the procedure described by Yang et al. (J. Am. Chem. Soc., 1996, 118, 9557). hydroxylamine hydrochloride (7.10 g, 0.102 mol) and sodium sulfate (40 g, 0.28 mol) were taken up in 200 mL water and 10 mL 2 M aqueous hydrochloric acid in a 1 L round-bottomed flask, and 1-acetyl-7-amino-2,3-dihydro-(1H)-indole (5.0 g, 28 mmol) was added. Chloral hydrate (5.63 g, 34.0 mmol) was then added, and the flask covered with a rubber septum and nitrogen balloon and heated at 55° C. overnight. After cooling to room temperature, the Intermediate 6 was collected by filtration and dried under vacuum to give product of sufficient purity that it could be used in the next step (5.74 g, 82% yield): ¹H NMR (400 MHz, DMSO-D₆) δ 2.30 (s, 3H) 3.07 (t, J=8.0 Hz, 2H) 4.13 (t, J=7.8 Hz, 2H) 7.09 (dd, J=7.3, 1.3 Hz, 1H) 7.14 (t, 1H) 7.48 (s, 1H) 7.73 (d, J=7.8 Hz, 1H) 10.76 (s, 1H) 12.33 (s, 1H).

### Intermediate 7: 8-acetyl-1,6,7,8-tetrahydro-1,8-diaza-as-indacene-2,3-dione

The cyclization step was carried out as described by Marvel and Hiers (Org. Synth. Coll. Vol. I, 327). In a 125 mL Erlenmeyer flask, 20 mL concentrated sulfuric acid was heated to 55° C. The isonitrosoacetanilide 6 was then added in small portions, with stirring, keeping the temperature of the solution below 70° C. Upon completion of the addition, the reaction mixture was heated at 80° C. for an additional 10 minutes, then cooled to room temperature and poured onto 100 mL crushed ice. It was allowed to stand for ±2 hour, and then the precipitate was collected by filtration, washing with water (3×), and dried under vacuum to give Intermediate 7 as a bright red, crystalline solid, of sufficient purity to be used in the next step (2.49 g, 46% yield): ¹H NMR (400 MHz, DMSO-D₆) δ 2.24 (s, 3H) 3.20 (t, J=8.3 Hz, 2H) 4.15 (t, J=8.3 Hz, 2H) 7.02 (d, J=7.3 Hz, 1H) 7.32 (d, J=7.6 Hz, 1H) 10.22 (s, 1H).

### 8-(4-chlorobenzyl)-7-hydroxy-2,3-dihydro-1H-pyrrolo[3,2-h]quinoline-6-carboxylic acid (Compound 4)

This compound was synthesized by the procedure described above for Compound 1, by reacting 8-acetyl-1,6,7,8-tetrahydro-1,8-diaza-as-indacene-2,3-dione, -Intermediate 7 (1.20 g, 5.21 mmol) with 3-(4-chlorophenyl)-2-oxopropyl acetate, Intermediate 2 (1.48 g, 6.52 mmol). The crude product was purified by flash chromatography over silica gel, eluting with 70 ethyl acetate:5 acetonitrile:2.5 methanol:2.5 water (+0.5% triethylamine), and lyophilized to yield the pure triethylammonium salt. To convert the salt back to the free acid form, it was taken up in 1:1 acetonitrile/water, acidified with concentrated hydrochloric acid, and then diluted with additional water to 20% acetonitrile in water. The acid was further purified by triturating with boiling ethanol to give pure Compound 4 as a beige powder (0.249 g, 13% yield): ¹H NMR (400 MHz, DMSO-D₆) δ 3.27 (t, J=8.1 Hz, 2H) 3.75 (t, J=8.1 Hz, 2H) 4.27 (s, 2H) 7.36 (m, 5H) 8.77 (s, 1H); HRMS (ESI+) calcd for C₁₉H₁₆ClN₂O₃ (MH⁺) 355.0844, found 355.0846.

### Example 5

### Preparation of 8-(4-chlorobenzyl)-7-hydroxy-2,3-dihydro-1H-9-aza-cyclopenta[a]naphthalene-6-carboxylic acid (Compound 5)

### Compound 5 was Prepared According to Scheme 5 Below:

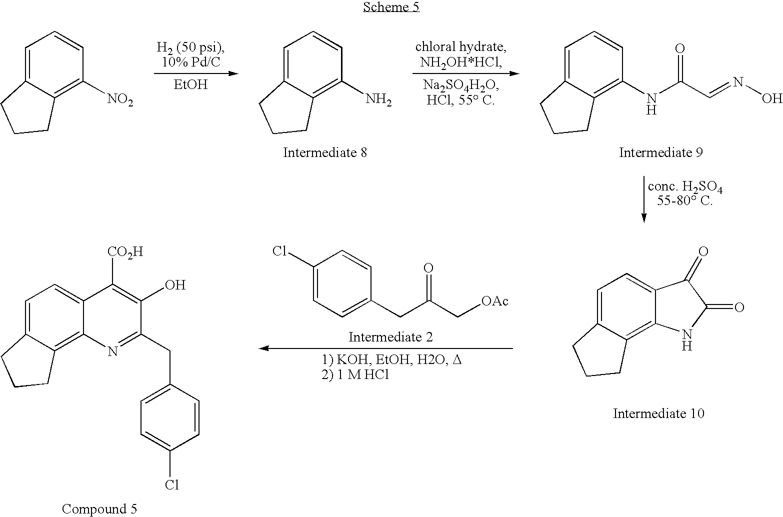

### Intermediate 8: 4-aminoindane

In a 500 mL Parr shaker vessel, 4-nitroindane (10 g, 61 mmol) was dissolved in 50 mL ethanol. A slurry of 10% Pd/C (1 g) in ethanol was added. The mixture was then placed on a Parr shaker under a hydrogen atmosphere (50 psi) for 1 hour, at which point t.l.c. (20% ethyl acetate in hexanes) showed that all the starting material had disappeared. To work up the reaction, the mixture was filtered twice through Celite, washing with a large amount of ethanol, and once through filter paper. The ethanol was evaporated under reduced pressure, and the crude product purified by flash chromatography over silica gel (10% ethyl acetate in hexanes) to give 8 as a viscous, faintly colored oil (7.04 g, 86% yield): ¹H NMR (400 MHz, DMSO-D₆) δ 1.95 (m, 2H) 2.61 (t, J=7.3 Hz, 2H) 2.76 (t, J=7.5 Hz, 2H) 4.77 (s, 2H) 6.36 (d, J=7.8 Hz, 1H) 6.42 (d, J=6.8 Hz, 1H) 6.80 (t, J=7.6 Hz, 1H).

### Intermediate 9: 2-hydroxyimino-N-indan-4-yl acetamide

This was synthesized according to the procedure described above for Intermediate 6. Intermediate 9 was prepared by reacting 4-aminoindane 8, (7.04 g, 52.9 mmol) with chloral hydrate (10.5 g, 63.4 mmol) and hydroxylamine hydrochloride (13.2 g, 0.190 mol) in the presence of sodium sulfate (75 g, 0.53 mol). Pure intermediate 9 was obtained as a brown solid (7.18 g, 66% yield): ¹H NMR (400 MHz, DMSO-D₆) δ 2.00 (m, 2H) 2.80 (t, J=7.3 Hz, 2H) 2.88 (t, J=7.6 Hz, 2H) 7.05 (d, J=6.8 Hz, 1H) 7.12 (t, J=7.6 Hz, 1H) 7.45 (d, J=7.8 Hz, 1H) 7.71 (s, 1H) 9.49 (s, 1H) 12.19 (s, 1H).

### Intermediate 10: 1,6,7,8-tetrahydro-1-aza-as-indacene-2,3-dione

The cyclization step was also carried out as described for Intermediate 7. However, after pouring the cooled reaction mixture onto ice, only a very small amount of precipitate appeared, even after chilling the mixture overnight. Thus, this black precipitate was filtered out and thrown away (<200 mg was isolated in this fashion), and the filtrate extracted into ethyl acetate (3×). The ethyl acetate solution was washed with brine, dried over anhydrous magnesium sulfate, filtered, and evaporated under reduced pressure to yield pure isatin 10 as a bright orange powder (0.36 g, 5.5% yield): ¹H NMR (400 MHz, DMSO-D₆) δ 2.07 (m, 2H) 2.76 (t, J=7.5 Hz, 2H) 2.88 (t, J=7.5 Hz, 2H) 6.95 (d, J=7.6 Hz, 1H) 7.30 (d, J=7.6 Hz, 1H) 11.10 (s, 1H).

### 8-(4-chlorobenzyl)-7-hydroxy-2,3-dihydro-1H-9-aza-cyclopenta[a]naphthalene-6-carboxylic acid (Compound 5)

This compound was synthesized by the procedure described above for Compound 1, reacting 1,6,7,8-tetrahydro-1-aza-as-indacene-2,3-dione 10 (0.36 g, 1.92 mmol) with Intermediate 2 (0.54 g, 2.40 mmol). The crude acid was purified as described above for Compound 4 to give pure product Compound 5 as a bright yellow powder (94 mg, 14% yield): ¹H NMR (400 MHz, DMSO-D₆) δ 2.15 (quint., 2H) 3.05 (t, J=7.3 Hz, 2H) 3.28 (t, J=7.5 Hz, 2H) 4.32 (s, 2H) 7.33 (s, 4H) 7.49 (d, J=8.3 Hz, 1H) 8.36 (d, J=8.1 Hz, 1H); HRMS (ESI+) calcd for C₂₀H₁₇ClNO₃ (MH⁺) 354.0892, found 354.0898.

### Example 6

### Preparation of 3-hydroxy-2-(4-trifluoromethoxybenzyl)-7,8,9,10-tetrahydrobenzo[h]quinoline-4-carboxylic acid (Compound 6)

### Compound 6 was Prepared According to Scheme 6 Below:

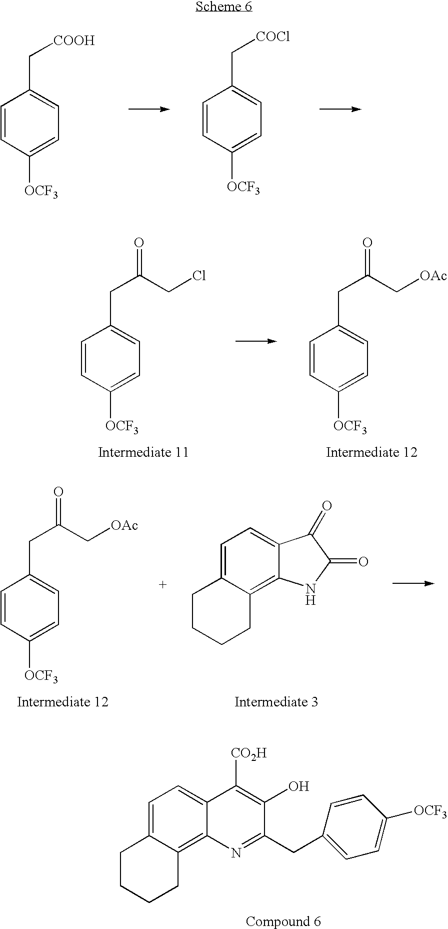

### Intermediate 11: 1-chloro-3-(4-trifluoromethoxy phenyl)propan2-one

A solution of 14.58 g (66.23 mmol) of 4-trifluoromethoxy phenyl acetic acid in 75 mL thionyl chloride was refluxed 1.5 hours, cooled, and the excess reagent was evaporated in vacuo. The resulting crude acid chloride was re-evaporated twice from dry toluene and used as such in the following step. To 175 mL diazomethane in Et₂O (ca. 0.57 mmol/mL) in an ice bath was added over 30 minutes a solution of the crude acid chloride in 85 mL Et₂O. The reaction was stirred 2 hours in the cold, then overnight at room temperature. Through the cooled (0° C.) solution was passed a gentle stream of Cl₂ gas for 5 minutes. After one more hour in the ice bath the reaction was diluted with 500 mL Et₂O, poured into 350 mL crushed ice-water, and the layers were separated. The aqueous layer was extracted with a second portion of Et₂O. The organic phases were washed with 5% NAHCO₃ (2×200 mL) and semi-saturated brine (400 mL), combined, dried (Na₂SO₄), and evaporated in vacuo. The residue was dissolved in 30 mL CH₂Cl₂, and the solution purified by flash chromatography on silica gel 60 (Merck) using EtOAc-cyclohexane 20:80 and 30:70 as the eluent. Pooling and evaporation of the appropriate fractions gave 6.97 g (44.1% overall) of the intermediate 11 as a colorless oil. ¹H NMR (400 MHz, CDCl₃)δ ppm 3.85 (s, 2H) 4.12 (s, 2H) 7.18 (m, J=21.98 Hz, 4H).

### Intermediate 12: acetic acid 2-oxo-3-(4-trifluoromethoxyphenyl) propyl ester

To a stirred, gently refluxing solution of the chloride 11 (6.80 g, 26.92 mmol) in 50 mL EtOH was added in one portion 5.68 g 29.6 mmol, 1.1 equiv.) CsOAc dissolved in 25 mL water and 2.5 mL glacial HOAc, and the reaction was refluxed 3 hours longer. Most of the EtOH was evaporated in vacuo, the concentrate was diluted with 100 mL water and the mixture extracted with EtOAc (2×400 mL). The organic phases were washed in sequence with ice cold, semi saturated NaHCO₃ (300 mL) and semi saturated brine (300 mL), combined, dried (Na₂SO₄), and evaporated in vacuo. The residue was crystallized from Et2O and excess hexanes to afford 3.15 g of 12 (42.4%) of the acetate as colorless flakes. (More product present in the mother liquors). ¹H NMR (400 MHz, CDC₃) δ2.16 (s, 3H) 3.75 (s, 2H) 4.71 (s, 2H) 7.23 (m, 4H).

### 3-hydroxy-2-(4-trifluoromethoxybenzyl)-7,8,9,10-tetrahydrobenzo[h]quinoline-4-carboxylic acid (Compound 6)

To 1.00 g (4.97 mmol) Intermediate 3 dissolved in 9 mL 6N KOH at 100-2° C. was added over one hour in several portions under stirring a solution of 2.26 g (8.18 mmol, 1.65 equiv.) acetate 12 in 18 mL lukewarm EtOH. At the end of the addition the solution was stirred one hour longer under gentle reflux, cooled, slowly diluted with 150 mL water, then acidified with 35 mL 2.5N HCl, added dropwise over 1.5 hours. The gummy precipitate was separated from the clear supernatant (pH<0) by decantation after standing 2 hours. The gum was dissolved in 600 mL EtOAc, the resulting solution was washed with 200 mL semi saturated brine, dried (Na₂SO₄), and evaporated in vacuo. Separation of the quinoline salicylate from unreacted cyclohexylisatin (27% recovery) and a variety of other impurities could only be achieved by gravity chromatography on silica gel 60 (Merck) of the triethylammonium salt, using a gradient of EtOAc-MeCN-MeOH—H₂O 70:5:2.5:2.5 to 70:10:5:5, containing 0.5% Net₃. Pooling of the appropriate fractions afforded pure product as the partial Net₃ salt. The salt was the converted to the free acid by treatment with 1 N HCl (aqueous) in a diluted EtOAc solution, which was quickly washed with semi saturated brine, dried, and evaporated in vacuo. Crystallization of the residue by slurring with a small volume of EtOAc-MeCN-MeOH—H₂O 70:10:5:5 (no Net₃) afforded 566 mg (27.3%) of the canary yellow quinoline salicilate as the free acid Compound 6. ¹H NMR (400 MHz, DMSO-D₆) δ1.81 (m, 4H) 2.83 (t, J=5.56 Hz, 2H) 3.13 (T, J=5.56 Hz, 2H) 4.35 (s, 2H) 7.28 (t, J=7.71 Hz, 3H) 7.45 (d, J=8.34 Hz, 2H) 8.21 (d, J=8.84 Hz, 1H).

### Preparation of 2-(3,4-dichlorobenzyl)-3-hydroxy-7,8,9,10-tetrahydrobenzo[h]quinoline-4-carboxylic acid (Compound 7)

### Compound 7 was Prepared According to Scheme 7 Below:

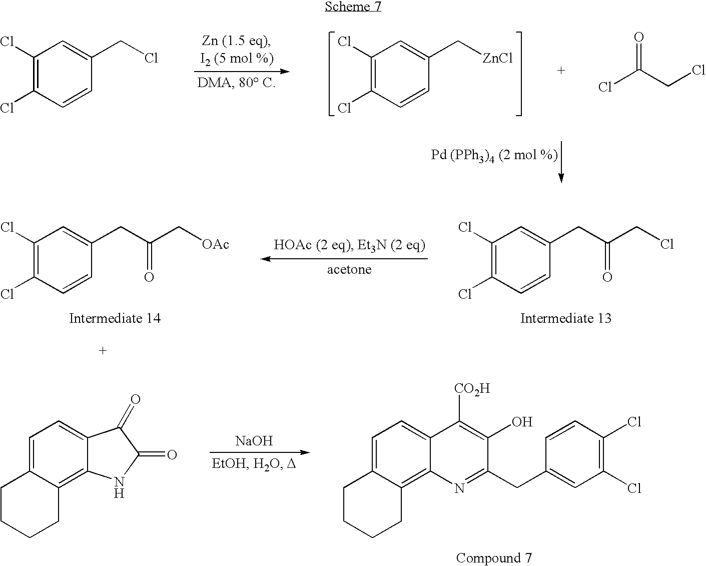

### Intermediate 13: 1-chloro-3-(3,4-dichlorophenyl)propan-2-one

The organozinc species was generated as described by S. Huo (Organic Letters 2003, 5 (4), 423-5). In a flame-dried 25 mL 2-necked round-bottomed flask, under an inert atmosphere, iodine (65 mg, 0.26 mmol) was taken up in 6 mL anhydrous N,N-dimethylacetamide. Zinc dust (0.502 g, 7.67 mmol) was added, and the suspension stirred until the red color of the iodine disappeared. Then, 3,4-dichlorobenzyl chloride (0.71 mL, 1.0 g, 5.1 mmol) was added via syringe, and the mixture heated at 80° C. until the t.l.c. of a hydrolyzed aliquot (5% ethyl acetate in hexanes, visualized by cerium molybdate staining) showed that the starting material had been consumed. The reaction vessel was placed in a water bath to cool it, and Pd(PPh₃)₄ (0.118 g, 0.102 mmol) was added, followed by dropwise addition, via syringe, of chloroacetyl chloride (0.61 mL, 0.87 g, 7.7 mmol). The brown suspension was allowed to stir overnight at room temperature. To work up the reaction, 12 mL 1 M HCl was added, and the mixture extracted into ethyl acetate (4×12 mL). The combined organic layers were washed with brine, dried over anhydrous MgSO₄, filtered, and evaporated. The crude product was purified by flash chromatography over silica gel (1-30% ethyl acetate in hexanes), to give material of sufficient purity to be used in the next step (0.545 g, 45% yield): ¹H NMR (400 MHz, CDCl₃) δ 3.89 (s, 2H) 4.13 (s, 2H) 7.06 (dd, J=8.2, 2.6 Hz, 1H) 7.33 (d, J=2.0 Hz, 1H) 7.42 (d, J=8.3 Hz, 1H).

### Intermediate 14: 3-(3,4-dichlorophenyl)-2-oxopropyl acetate

In a round-bottomed flask, 1-chloro-3-(3,4-dichlorophenyl)propan-2-one (0.545 g, 2.30 mmol) was taken up in 2 mL acetone, and acetic acid (0.26 mL, 0.28 g, 4.6 mmol) was added. The solution was cooled in an ice water bath, and triethylamine (0.64 mL, 0.47 g, 4.6 mmol) added dropwise via syringe over 30 minutes. The reaction mixture was then stirred overnight. Precipitated triethylammonium chloride was removed by filtration, and the filtrate was evaporated, taken up in 10 mL ethyl acetate, washed twice with brine, dried over anhydrous MgSO₄, filtered, and evaporated. The crude product was purified by flash chromatography over silica gel (10-30% ethyl acetate in hexanes) to give a pure product (0.200 g, 33% yield): ¹H NMR (400 MHz, CDCl₃) δ 2.17 (s, 3H) 3.71 (s, 2H) 4.71 (s, 2H) 7.05 (dd, J=8.2, 2.2 Hz, 1H) 7.32 (d, J=2.0 Hz, 1H) 7.41 (d, J=8.1 Hz, 1H).

### 2-(3,4-dichlorobenzyl)-3-hydroxy-7,8,9,10-tetrahydrobenzo[h]quinoline-4-carboxylic acid (Compound 7)

The Pfitzinger reaction was used. In a 2-necked 25 mL round-bottomed flask, 6,7,8,9-tetrahydrobenzo[g]indoline-2,3-dione (0.119 g, 0.590 mmol) was taken up in 1 mL ethanol and 3 mL 10 M NaOH, and the mixture heated to reflux temperature. A solution of 3-(3,4-dichlorophenyl)-2-oxopropyl acetate (0.200 g, 0.767 mmol) in 3 mL ethanol was added in small portions over the course of 1 hour, by syringe. Refluxing was continued for an additional hour after the addition was complete, and the reaction mixture was then cooled to room temperature and acidified with glacial acetic acid, and the yellow precipitate collected by filtration. This crude product was purified by preparative HPLC (acetonitrile/water/triethylamine), and the pure salt thus obtained was converted back to the free acid by acidification of a 5% acetonitrile in water solution with concentrated HCl. The bright yellow precipitate was collected by filtration and dried under vacuum (47.8 mg, 20% yield): ¹H NMR (400 MHz, DMSO-d₆) δ 1.73-1.86 (m, 4H) 2.81 (t, J=6.1 Hz, 2H) 3.12 (t, J=5.9 Hz, 2H) 4.30 (s, 2H) 7.28 (t, J=8.7 Hz, 2H) 7.53 (d, J=8.1 Hz, 1H) 7.59 (d, J=2.0 Hz, 1H) 8.19 (d, J=8.6 Hz, 1H); HRMS (ESI+) calcd for C₂₁H₁₈Cl₂NO₃ (MH⁺) 402.0658, found 402.0661.

### Example 8

### Preparation of 3-hydroxy-2-(thiophen-2-ylmethyl)-7,8,9,10-tetrahydrobenzo[h]quinoline-4-carboxylic acid (Compound 8)

### Compound 8 was Prepared According to Scheme 8 Below:

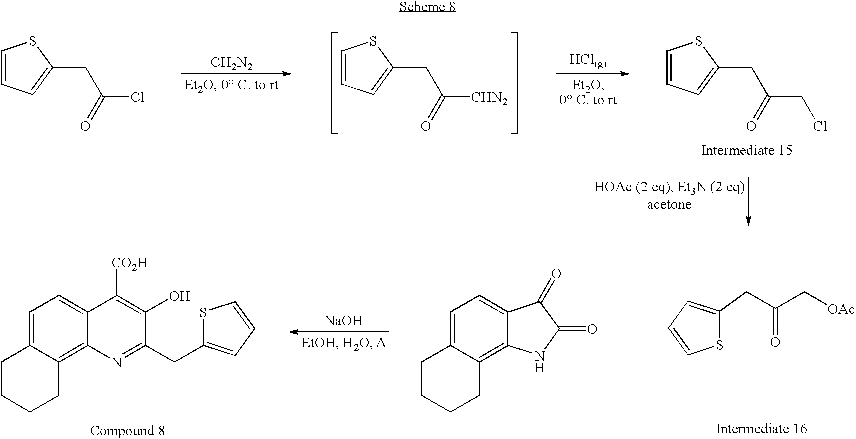

### Intermediate 15: 1-chloro-3-(thiophen-2-yl)propan-2-one

The chloride was synthesized by Arndt-Eistert homologation of the acid chloride. A solution of 2-thiopheneacetyl chloride (3.8 mL, 5.0 g, 31 mmol) in 60 mL ether was added dropwise, with stirring, from an addition funnel to a 1 L Erlenmeyer flask containing 85 mL of an ethereal diazomethane solution, cooled in an ice water bath. Upon completion of the addition (which was done over 30 minutes), the solution was allowed to stir overnight, gradually warming to room temperature. It was then cooled in an ice water bath once again, and a gentle stream of dry HCl gas was passed through, until nitrogen evolution ceased. The mixture was stirred for 1 hour, then poured into 150 mL ice water, stirred for 20 minutes, and extracted twice into 180 mL portions of ether. The combined ether extracts were washed with 5% Na₂CO₃ (150 mL) and brine (120 mL), then dried over anhydrous MgSO₄, filtered, and evaporated. Purification by flash chromatography over silica gel (5% ethyl acetate in hexanes) gave a clear, yellow oil, which turned into a black solid upon standing overnight, unless it was stored in the freezer, under nitrogen (2.33 g, 43% yield): ¹H NMR (400 MHz, CDCl₃) δ 4.11 (s, 2H) 4.17 (s, 2H) 6.93-6.96 (m, 1H) 7.00 (dd, J=5.2, 3.4 Hz, 1H) 7.24-7.28 (m, 1H).

### Intermediate 16: 3-(thiophen-2-yl)-2-oxopropyl acetate

The procedure described above for the synthesis of 3-(3,4-dichlorophenyl)-2-oxopropyl acetate was followed, reacting 1-chloro-3-(thiophen-2-yl)propan-2-one (1.00 g, 5.73 mmol) with acetic acid (0.66 mL, 0.69 g, 12 mmol) and triethylamine (1.60 mL, 1.16 g, 11.5 mmol). Purification by flash chromatography over silica gel (10-40% ethyl acetate in hexanes) gave an orange oil (0.144 g, 13% yield): ¹H NMR (400 MHz, CDCl₃) δ 2.17 (s, 3H) 3.95 (s, 2H) 4.74 (s, 2H) 6.92-6.94 (m, 1H) 6.99 (dd, J=5.2, 3.4 Hz, 1H) 7.25 (dd, J=5.1, 1.3 Hz, 1H).

### 3-hydroxy-2-(thiophen-2-ylmethyl)-7,8,9,10-tetrahydrobenzo[h]quinoline-4-carboxylic acid (Compound 8)

The procedure described above for the synthesis and purification of example 7 was followed, reacting 6,7,8,9-tetrahydrobenzo[g]indoline-2,3-dione (0.112 g, 0.557 mmol) with 3-(thiophen-2-yl)-2-oxopropyl acetate (0.144 g, 0.724 mmol). Product was obtained as a dark yellow powder (9.1 mg, 4.8% yield): ¹H NMR (400 MHz, DMSO-d₆) δ 1.75-1.88 (m, 4H) 2.83 (t, J=5.7 Hz, 2H) 3.17-3.25 (m, 2H) 4.49 (s, 2H) 6.89-6.94 (m, 1H) 6.94-6.98 (m, 1H) 7.27 (d, J=9.1 Hz, 1H) 7.32 (dd, J=5.3, 1.3 Hz, 1H) 8.18 (d, J=8.8 Hz, 1H); HRMS (ESI+) calcd for C₁₉H₁₈NO₃S (MH⁺) 340.1002, found 340.1011.

### Example 9

### Preparation of 2-(benzo[b]thiophen-3-ylmethyl)-3-hydroxy-7,8,9,10-tetrahydrobenzo[h]quinoline-4-carboxylic acid (Compound 9)

### Compound 9 was Prepared According to Scheme 9 Below:

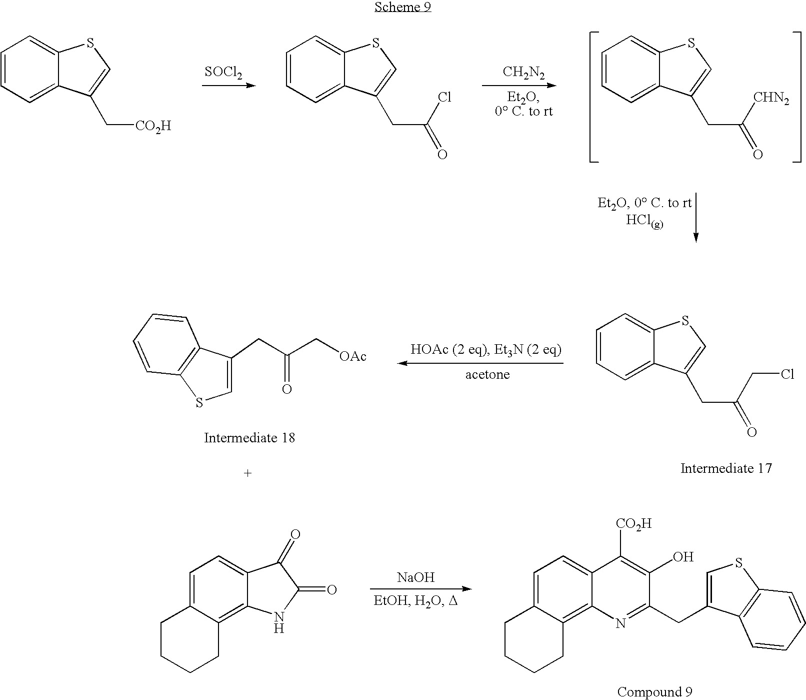

### Intermediate 17: 1-(benzo[b]thiophen-3-yl)-3-chloropropan-2-one

The procedure described above for the synthesis of 1-chloro-3-(thiophen-2-yl)propan-2-one was followed. To prepare the acid chloride, 2-(benzo[b]thiophen-3-yl)acetic acid (1.00 g, 5.20 mmol) was added to 6 mL thionyl chloride in a 25 mL round-bottomed flask. The mixture was stirred overnight at room temperature, and the thionyl chloride then removed in vacuo and the residue azeotroped twice with toluene. The acid chloride was then reacted with diazomethane and HCl.

The crude product was purified by flash chromatography over silica gel (2-30% ethyl acetate in hexanes) to give pure material (0.661 g, 56% yield): ¹H NMR (400 MHz, CDCl₃) δ 4.12 (s, 2H) 4.14 (d, J=1.0 Hz, 2H) 7.36-7.44 (m, 3H) 7.67-7.71 (m, 1H) 7.87-7.90 (m, 1H)

### Intermediate 18: 3-(benzo[b]thiophen-3-yl)-2-oxopropyl acetate

The procedure described above for the synthesis of 3-(3,4-dichlorophenyl)-2-oxopropyl acetate was followed, reacting 1-(benzo[b]thiophen-3-yl)-3-chloropropan-2-one (0.661 g, 2.94 mmol) with acetic acid (0.34 mL, 0.35 g, 5.9 mmol) and triethylamine (0.82 mL, 0.59 g, 5.9 mmol). Flash chromatography over silica gel (10-40% ethyl acetate in hexanes) gave pure product (0.372 g, 51% yield): ¹H NMR (400 MHz, CDCl₃) δ 2.14 (s, 3H) 3.98 (s, 2H) 4.71 (s, 2H) 7.34-7.44 (m, 3H) 7.67-7.70 (m, 1H) 7.86-7.89 (m, 1H).

### 2-(benzo[b]thiophen-3-ylmethyl)-3-hydroxy-7,8,9,10-tetrahydrobenzo[h]quinoline-4-carboxylic acid (Compound 9)

The procedure described above for the synthesis and purification of Compound 7 was followed, reacting 6,7,8,9-tetrahydrobenzo[g]indoline-2,3-dione (0.232 g, 1.15 mmol) with 3-(benzo[b]thiophen-3-yl)-2-oxopropyl acetate (0.372 g, 1.50 mmol). Product was obtained as a bright yellow powder (30.6 mg, 6.8% yield): ¹H NMR (400 MHz, DMSO-d₆) δ 1.71-1.85 (m, 4H) 2.80 (t, J=5.2 Hz, 2H) 3.11 (t, J=5.1 Hz, 2H) 4.53 (s, 2H) 7.25 (d, J=8.8 Hz, 1H) 7.30-7.45 (m, 3H) 7.94 (d, J=7.8 Hz, 1H) 8.11 (d, J=8.1 Hz, 1H) 8.19 (d, J=8.6 Hz, 1H); HRMS (ESI+) calcd for C₂₃H₂₀NO₃S (MH⁺) 390.1159, found 390.1167.

### Example 10

### Preparation of 2-(2-chlorobenzyl)-3-hydroxy-7,8,9,10-tetrahydrobenzo[h]quinoline-4-carboxylic acid (Compound 10)

### Compound 10 was Prepared According to Scheme 10 Below:

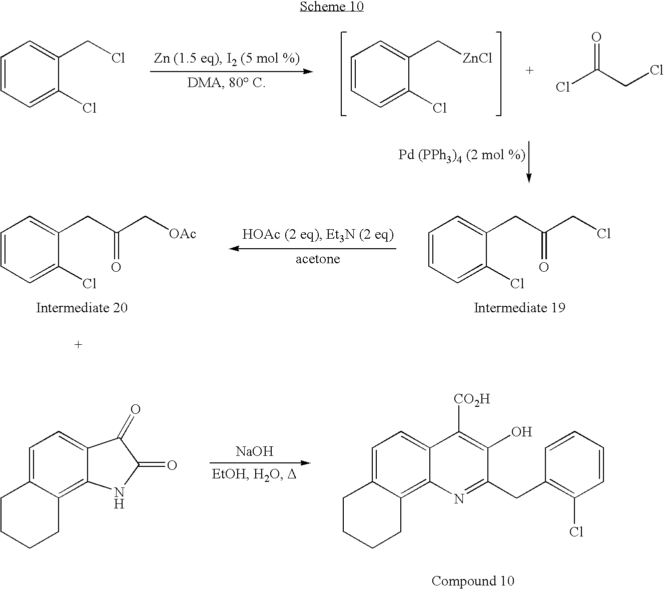

### Intermediate 19: 1-chloro-3-(2-chlorophenyl)propan-2-one

The procedure described above for the synthesis of 1-chloro-3-(3,4-dichlorophenyl)propan-2-one was followed, reacting 2-chlorobenzyl chloride (1.6 mL, 2.0 g, 12 mmol) with zinc dust (1.22 g, 18.6 mmol) in the presence of iodine (0.157 g, 0.620 mmol), then with chloroacetyl chloride (1.5 mL, 2.1 g, 19 mmol) in the presence of Pd(PPh₃)₄ (0.287 g, 0.248 mmol). Flash chromatography over silica gel (10% ethyl acetate in hexanes) gave product of sufficient purity to be used in the next step (0.556 g, 22% yield): ¹H NMR (400 MHz, CDCl₃) δ 4.03 (s, 2H) 4.19 (s, 2H) 7.19-7.29 (m, 3H) 7.38-7.42 (m, 1H).

### Intermediate 20: 3-(2-chlorophenyl)-2-oxopropyl acetate

The procedure described above for the synthesis of 3-(3,4-dichlorophenyl)-2-oxopropyl acetate was followed, reacting 1-chloro-3-(2-chlorophenyl)propan-2-one (0.556 g, 2.74 mmol) with acetic acid (0.31 mL, 0.33 g, 5.5 mmol) and triethylamine (0.76 mL, 0.56 g, 5.5 mmol). Flash chromatography over silica gel (5-40% ethyl acetate in hexanes) gave pure product (0.251 g, 43% yield): ¹H NMR (400 MHz, CDCl₃) δ 2.17 (s, 3H) 3.88 (s, 2H) 4.75 (s, 2H) 7.24-7.27 (m, 3H) 7.38-7.42 (m, 1H).

### 2-(2-chlorobenzyl)-3-hydroxy-7,8,9,10-tetrahydrobenzo[h]quinoline-4-carboxylic acid (Compound 10)

The procedure described above for the synthesis and purification of example 7 was followed, reacting 6,7,8,9-tetrahydrobenzo[g]indoline-2,3-dione (0.183 g, 0.908 mmol) with 3-(2-chlorophenyl)-2-oxopropyl acetate (0.251 g, 1.18 mmol). Product was obtained as a bright yellow powder (79.5 mg, 24% yield): ¹H NMR (400 MHz, DMSO-d₆) δ 1.74 (br. s, 4H) 2.80 (br. s, 2H) 2.92 (br. s, 2H) 4.42 (s, 2H) 7.22-7.32 (m, 4H) 7.43-7.50 (m, 1H) 8.23 (d, J=8.8 Hz, 1H); HRMS (ESI+) calcd for C₂₁H₁₉ClNO₃ (MH⁺) 368.1048, found 368.1047.

### Example 11

### Preparation of 2-(3-chlorobenzyl)-3-hydroxy-7,8,9,10-tetrahydrobenzo[h]quinoline-4-carboxylic acid (Compound 11)

### Compound 11 was Prepared According to Scheme 11 Below:

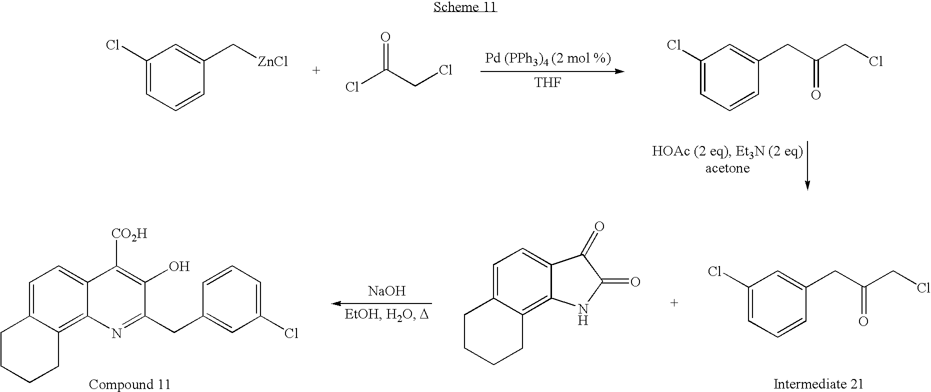

### Intermediate 21: 3-(3-chlorophenyl)-2-oxopropyl acetate

A flame-dried 50 mL round-bottomed flask, under an inert atmosphere, was charged with Pd(PPh₃)₄ (0.30 g, 0.26 mmol). Anhydrous THF (7 mL) was added, then a 0.5 M THF solution of 3-chlorobenzylzinc chloride (26 mL, 13 mmol). The flask was cooled in an ice bath, and chloroacetyl chloride was added via syringe, over 1 hour. The solution went from a very dark brown (almost black), to a clear, light yellow. The mixture was stirred overnight at room temperature, then quenched by addition of 5 g ice, stirred for an additional hour, diluted with ethyl acetate, washed twice with brine, dried over anhydrous MgSO₄, filtered, and evaporated.

This crude material was reacted with acetic acid (1.42 mL, 1.49 g, 24.8 mmol) and triethylamine (3.46 mL, 2.51 g, 24.8 mmol), as described above for the synthesis of 3-(3,4-dichlorophenyl)propan-2-one. Flash chromatography over silica gel (20% ethyl acetate in hexanes) gave pure product (1.22 g, 46% yield): ¹H NMR (400 MHz, CDCl₃) δ 2.16 (s, 3H) 3.72 (s, 2H) 4.69-4.71 (m, 2H) 7.08-7.11 (m, 1H) 7.21-7.23 (m, 1H) 7.26-7.29 (m, 2H).

### 2-(3-chlorobenzyl)-3-hydroxy-7,8,9,10-tetrahydrobenzo[h]quinoline-4-carboxylic acid (Compound 11)

The procedure described above for the synthesis and purification of example 7 was followed, reacting 6,7,8,9-tetrahydrobenzo[g]indoline-2,3-dione (0.495 g, 2.46 mmol) with 3-(3-chlorophenyl)-2-oxopropyl acetate (0.680 g, 3.20 mmol). Product was obtained as a bright yellow powder (186 mg, 20% yield): ¹H NMR (400 MHz, DMSO-d₆) δ 1.74-1.88 (m, 4H) 2.83 (t, J=4.3 Hz, 2H) 3.15 (t, J=4.6 Hz, 2H) 4.32 (s, 2H) 7.24-7.35 (m, 4H) 7.39 (s, 1H) 8.20 (d, J=8.8 Hz, 1H); HRMS (ESI+) calcd for C₂₁H₁₉ClNO₃ (MH⁺) 368.1048, found 368.1046.

### Example 12

### Preparation of 3-hydroxy-2-[2-(3-methylbenzo[b]thiophen-2-ylmethyl)]-7,8,9,10-tetrahydrobenzo[h]quinoline-4-carboxylic acid (Compound 12)

### Compound 12 was Prepared According to Scheme 12 Below:

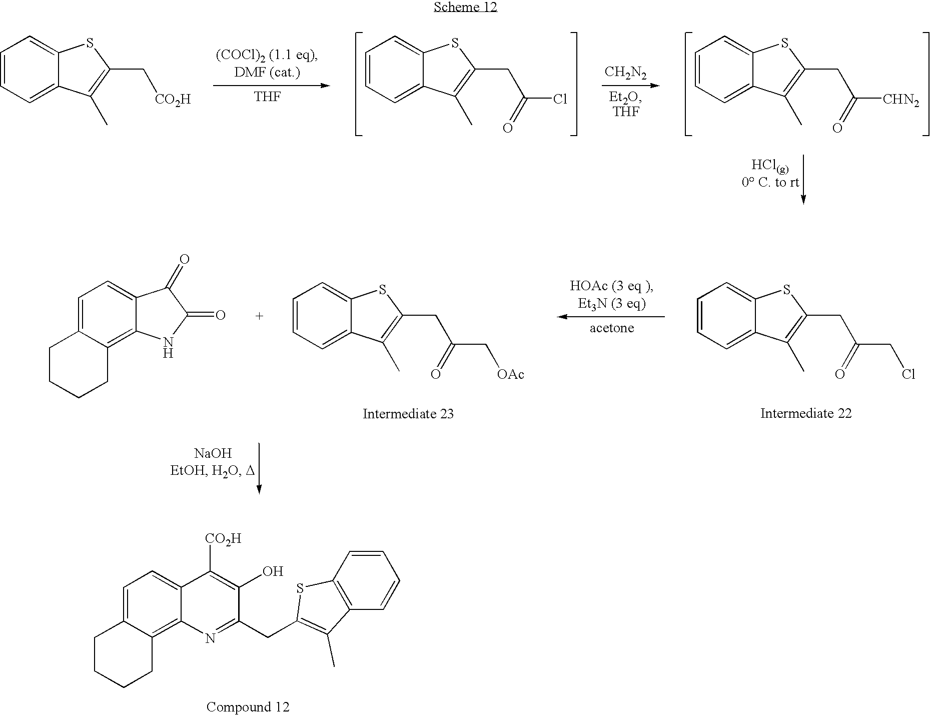

### Intermediate 22: 1-chloro-3-[2-(3-methylbenzo[b]thiophen-2-yl)propan-2-one

The procedure described above for the synthesis of 1-(benzo[b]thiophen-3-yl)-3-chloropropan-2-one was followed, except that in this case the acid chloride was generated by dropwise addition of oxalyl chloride (1.2 mL, 1.7 g, 13 mmol) to a cold THF solution (18 mL) of 2-(3-methylbenzo[b]thiophen-2-yl)acetic acid (2.5 g, 12 mmol), containing catalytic DMF. After the addition was complete, the solution was allowed to stir at room temperature for 1 hour, then added to an ethereal diazomethane solution, as previously described.

Work-up and purification by flash chromatography over silica gel (10% ethyl acetate in hexanes) gave product of sufficient purity to be used in the next step: ¹H NMR (400 MHz, CDCl₃) δ 2.35 (s, 3H) 4.13 (s, 2H) 4.17 (s, 2H) 7.30-7.42 (m, 2H) 7.67 (d, J=7.6 Hz, 1H) 7.79 (d, J=7.8 Hz, 1H).

### Intermediate 23: 3-[2-(3-methylbenzo[b]thiophen-2-yl)]-2-oxopropyl acetate

The procedure described above for the synthesis of 3-(3,4-dichlorophenyl)-2-oxopropyl acetate was followed, reacting 1-chloro-3-[2-(3-methylbenzo[b]thiophen-2-yl)propan-2-one (0.754 g, 3.16 mmol) with acetic acid (0.54 mL, 0.57 g, 9.5 mmol) and triethylamine (1.3 mL, 0.96 g, 9.5 mmol). Flash chromatography over silica gel (16-36% ethyl acetate in hexanes) gave pure product (0.109 g, 13% yield): ¹H NMR (400 MHz, CDCl₃) δ 2.17 (s, 3H) 2.35 (s, 3H) 3.97 (s, 2H) 4.73 (s, 2H) 7.31-7.41 (m, 2H) 7.64-7.68 (m, 1H) 7.76-7.80 (m, 1H).

### 3-hydroxy-2-[2-(3-methylbenzo[b]thiophen-2-ylmethyl)]-7,8,9,10-tetrahydrobenzo[h]quinoline-4-carboxylic acid (Compound 12)

The procedure described above for the synthesis of Compound 7 was followed, reacting 6,7,8,9-tetrahydrobenzo[g]indoline-2,3-dione (64 mg, 0.318 mmol) with 3-[2-(3-methylbenzo[b]thiophen-2-yl)]-2-oxopropyl acetate (0.109 g, 0.414 mmol). Preparative HPLC purification (water/acetonitrile/triethylamine), followed by lyophilization gave product as a fluffy, light yellow solid (186 mg, 20% yield): ¹H NMR (400 MHz, DMSO-d₆) δ 1.76-1.88 (m, 4H) 2.50 (s, 3H) 2.80 (t, J=5.3 Hz, 2H) 3.20 (t, J=5.8 Hz, 2H) 4.52 (s, 2H) 7.16 (d, J=8.8 Hz, 1H) 7.25 (t, J=7.6 Hz, 1H) 7.33 (t, J=7.6 Hz, 1H) 7.68 (d, J=7.8 Hz, 1H) 7.79 (d, J=8.1 Hz, 1H) 8.68 (s, 1H); HRMS (ESI+) calcd for C₂₄H₂₂NO₃S (MH⁺) 404.1315, found 404.1312.

### Example 13

### Preparation of 3-hydroxy-2-(thiophen-3-ylmethyl)-7,8,9,10-tetrahydrobenzo[h]quinoline-4-carboxylic acid (Compound 13)

### Compound 13 was Prepared According to Scheme 13 Below:

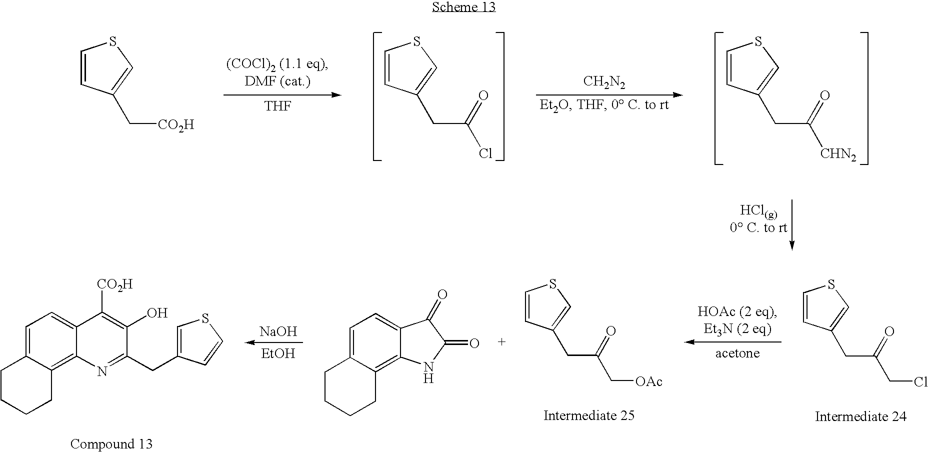

### Intermediate 24: 1-chloro-3-(thiophen-3-yl)propan-2-one

The procedure described above for the synthesis of 1-chloro-3-[2-(3-methylbenzo[b]thiophen-2-yl)propan-2-one was followed, reacting thiophene-3-acetic acid (5.32 g, 37.4 mmol) with oxalyl chloride (3.6 mL, 5.2 g, 41 mmol, then ethereal diazomethane, then dry HCl gas. Work-up gave pure product, a brown oil which solidified upon refrigeration to a golden-brown, waxy solid (6.52 g, 100% yield): ¹H NMR (400 MHz, CDCl₃) δ 3.94 (s, 2H) 4.13 (s, 2H) 6.99 (d, J=5.1 Hz, 1H) 7.16 (dd, J=1.5, 0.8 Hz, 1H) 7.33 (dd, J=4.9, 2.9 Hz, 1H).

### Intermediate 25: 2-oxo-3-(thiophen-3-yl)propyl acetate

The procedure described above for the synthesis of 3-(3,4-dichlorophenyl)-2-oxopropyl acetate was followed, reacting 1-chloro-3-(thiophen-3-yl)propan-2-one (6.53 g, 37.4 mmol) with acetic acid (4.3 mL, 4.5 g, 75 mmol) and triethylamine (10.4 mL, 7.57 g, 74.8 mmol). Flash chromatography over silica gel (20% ethyl acetate in hexanes) gave pure product, a golden-yellow oil (3.85 g, 52% yield): ¹H NMR (400 MHz, CDCl₃) δ 2.16 (s, 3H) 3.77 (s, 2H) 4.70 (s, 2H) 6.98 (dd, J=4.8, 1.3 Hz, 1H) 7.14 (dd, J=1.8, 1.0 Hz, 1H) 7.32 (dd, J=4.9, 2.9 Hz, 1H).

### 3-hydroxy-2-(thiophen-3-ylmethyl)-7,8,9,10-tetrahydrobenzo[h]quinoline-4-carboxylic acid (Compound 13)

The procedure described above for the synthesis and purification of example 7 was followed, reacting 6,7,8,9-tetrahydrobenzo[g]indoline-2,3-dione (0.500 g, 2.48 mmol) with 2-oxo-3-(thiophen-3-yl)propyl acetate (0.640 g, 3.23 mmol). Product was obtained as a bright yellow powder (187 mg, 22% yield): ¹H NMR (400 MHz, DMSO-d₆) δ 1.73-1.89 (m, 4H) 2.83 (t, J=4.9 Hz, 2H) 3.18 (t, J=5.7 Hz, 2H) 4.32 (s, 2H) 7.10 (d, J=4.8 Hz, 1H) 7.23 (s, 1H) 7.27 (d, J=8.8 Hz, 1H) 7.40-7.47 (m, 1H) 8.22 (d, J=8.6 Hz, 1H); HRMS (ESI+) calcd for C₁₉H₁₈NO₃S (MH⁺) 340.1002, found 340.1006. Anal. Calcd for C₁₉H₁₇NO₃S.2H₂O: C, 60.78; H, 5.64; N, 3.73. Found: C, 63.01; H, 5.60; N, 3.76.

### Example 14

### Preparation of 3-hydroxy-2-(indol-3-ylmethyl)-7,8,9,10-tetrahydrobenzo[h]quinoline-4-carboxylic acid (Compound 14)

### Compound 14 was Prepared According to Scheme 14 Below:

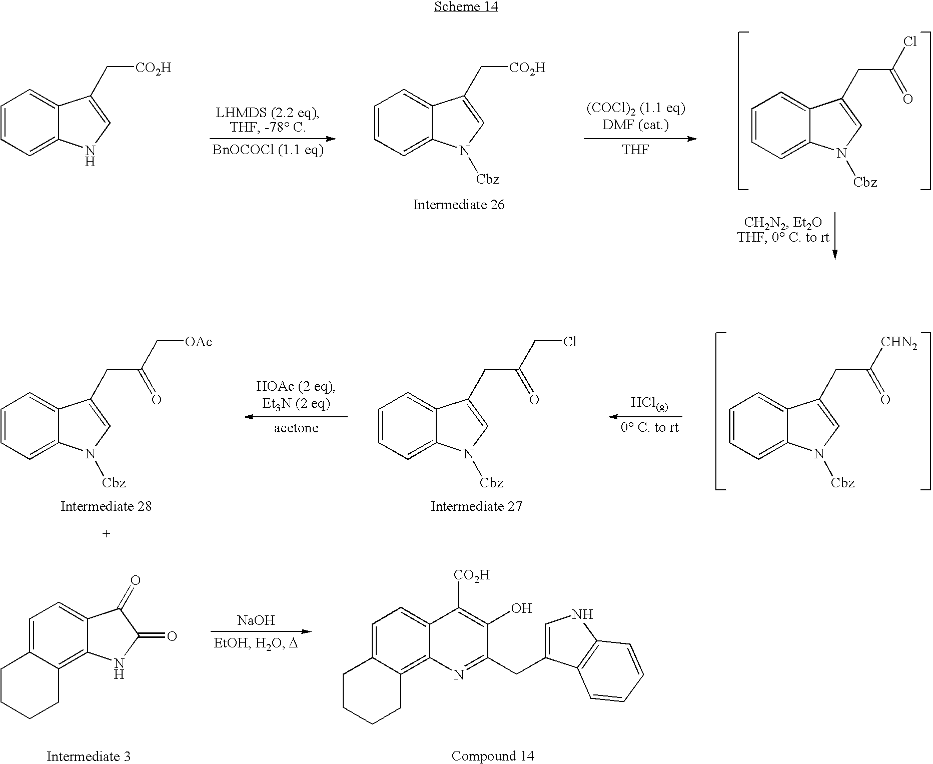

### Intermediate 26: 1-(benzyloxycarbonyl)indol-3-yl acetic acid

Indole-3-acetic acid (13 g, 74 mmol) was taken up in 130 mL anhydrous THF in a flame-dried, 2-necked 1 L round-bottomed flask, under an inert atmosphere, and cooled to −78° C. (dry ice/acetone bath). A 1.0 M THF solution of LHMDS (163 mL, 0.163 mol) was added via syringe over 30 minutes, and the reaction mixture allowed to stir for an additional 30 minutes at minus 78° C. once the addition was complete. Next, benzyl chloroformate (11.7 mL, 13.9 g, 81.6 mmol) was added dropwise via syringe. Stirring was then continued for 1 hour. To work up the reaction mixture, it was quenched with 2 M HCl, and partitioned between 2 M HCl and ethyl acetate. The aqueous layer was extracted with additional ethyl acetate, and the combined organic layers washed with brine, dried over anhydrous MgSO₄, filtered, and evaporated to give a white solid with a pinkish tinge (22.49 g, 98% yield): ¹H NMR (400 MHz, DMSO-d₆) δ 3.71 (s, 2H) 5.47 (s, 2H) 7.27 (t, J=7.2 Hz, 1H) 7.32-7.47 (m, 4H) 7.54 (d, J=6.8 Hz, 2H) 7.58 (d, J=7.6 Hz, 1H) 7.68 (s, 1H) 8.08 (d, J=8.1 Hz, 1H) 12.43 (s, 1H); HRMS (ESI+) calcd for C₁₈H₁₆NO₄ (MH⁺) 310.1074, found 310.1080.

### Intermediate 27: 3-[1-(benzyloxycarbonyl)indol-3-yl]-1-chloropropan-2-one

The procedure described above for the synthesis of 1-chloro-3-[2-(3-methylbenzo[b]thiophen-2-yl)propan-2-one was followed, reacting 1-(benzyloxycarbonyl)indol-3-yl acetic acid (22.49 g, 72.7 mmol) with oxalyl chloride (7.0 mL, 10 g, 80 mmol, then ethereal diazomethane, then dry HCl gas. Flash chromatography over silica gel (15-20% ethyl acetate in hexanes) gave pure product (21.64 g, 87% yield): ¹H NMR (400 MHz, CDCl₃) δ 3.97 (d, J=1.0 Hz, 2H) 4.15 (s, 2H) 5.45 (s, 2H) 7.27-7.30 (m, 1H) 7.33-7.51 (m, 7H) 7.63 (s, 1H) 8.19 (br. s, 1H); HRMS (ESI+) calcd for C₁₉H₁₇ClNO₃ (MH⁺) 342.0892, found 342.0900.

### Intermediate 28: 3-[1-(benzyloxycarbonyl)indol-3-yl]-2-oxopropyl acetate

The procedure described above for the synthesis of 3-(3,4-dichlorophenyl)-2-oxopropyl acetate was followed, reacting 3-[1-(benzyloxycarbonyl)indol-3-yl]-1-chloropropan-2-one (19.28 g, 56.4 mmol) with acetic acid (6.5 mL, 6.8 g, 0.11 mol) and triethylamine (15.7 mL, 11.4 g, 0.113 mol). Flash chromatography over silica gel (25% ethyl acetate in hexanes) gave pure product as an orange oil that solidified under vacuum to a yellow solid (9.06 g, 44% yield): ¹H NMR (400 MHz, CDCl₃) δ 2.15 (s, 3H) 3.81 (d, J=0.8 Hz, 2H) 4.73 (s, 2H) 5.45 (s, 2H) 7.26-7.30 (m, 1H) 7.32-7.51 (m, 7H) 7.62 (s, 1H) 8.18 (s, 1H).

### 3-hydroxy-2-(indol-3-ylmethyl)-7,8,9,10-tetrahydrobenzo[h]quinoline-4-carboxylic acid (Compound 14)

The procedure described above for the synthesis and purification of example 7 was followed, reacting 6,7,8,9-tetrahydrobenzo[g]indoline-2,3-dione (0.294 g, 1.46 mmol) with 3-[1-(benzyloxycarbonyl)indol-3-yl]-2-oxopropyl acetate (0.693 g, 1.90 mmol). Product was obtained as a brownish-orange powder (93 mg, 17% yield): ¹H NMR (400 MHz, DMSO-d₆) δ 1.65-1.93 (m, 4H) 2.83 (br. s, 2H) 3.24 (br. s, 2H) 4.41 (s, 2H) 6.90-7.08 (m, 2H) 7.13-7.36 (m, 3H) 7.75 (d, J=7.1 Hz, 1H) 8.19 (s, 1H) 10.84 (s, 1H); HRMS (ESI+) calcd for C₂₃H₂₁N₂O₃ (MH⁺) 373.1547, found 373.1548. Anal. Calcd for C₂₃H₂₀N₂O₃. H₂O: C, 70.75; H, 5.68; N, 7.17. Found: C, 71.04; H, 5.64; N, 7.01.

### Example 15

### Preparation of 2-(5-chlorobenzo[b]thiophen-3-ylmethyl)-3-hydroxy-7,8,9,10-tetrahydrobenzo[h]quinoline-4-carboxylic acid (Compound 15)

### Compound 15 was Prepared According to Scheme 15 Below:

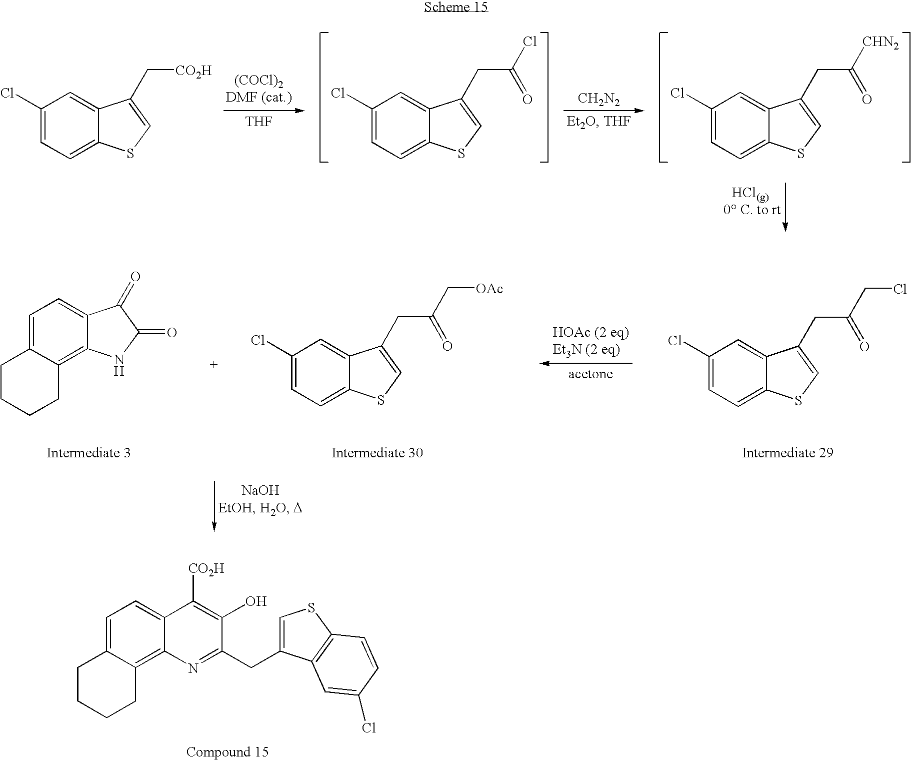

### Intermediate 29: 1-chloro-3-(5-chlorobenzo[b]thiophen-3-yl)-propan-2-one

The procedure described above for the synthesis of 1-chloro-3-[2-(3-methylbenzo[b]thiophen-2-yl)propan-2-one was followed, reacting 5-chlorobenzo[b]thiophen-3-yl acetic acid (4.00 g, 17.6 mmol) with oxalyl chloride (1.7 mL, 2.5 g, 19 mmol), then ethereal diazomethane, then dry HCl gas. Work-up of the reaction mixture gave pure product as a light golden-yellow solid (4.43 g, 97% yield): ¹H NMR (400 MHz, CDCl₃) δ 4.12 (s, 2H) 4.15 (s, 2H) 7.35 (dd, J=8.6, 2.1 Hz, 1H) 7.43 (s, 1H) 7.65 (d, J=2.1 Hz, 1H) 7.79 (d, J=8.6 Hz, 1H).

### Intermediate 30: 3-(5-chlorobenzo[b]thiophen-3-yl)-2-oxopropyl acetate

The procedure described above for the synthesis of 3-(3,4-dichlorophenyl)-2-oxopropyl acetate was followed, reacting 1-chloro-3-(5-chlorobenzo[b]thiophen-3-yl)-propan-2-one (4.43 g, 17.1 mmol) with acetic acid (2.0 mL, 2.1 g, 35 mmol) and triethylamine (4.9 mL, 3.6 g, 35 mmol). Flash chromatography over silica gel (20% ethyl acetate in hexanes) gave pure product, a pale yellow solid (2.76 g, 57% yield): ¹H NMR (400 MHz, CDCl₃) δ 2.16 (s, 3H) 3.94 (d, J=1.0 Hz, 2H) 4.73 (s, 2H) 7.34 (ddd, J=8.6, 2.0, 0.5 Hz, 1H) 7.39-7.42 (m, 1H) 7.65 (d, J=2.0 Hz, 1H) 7.78 (dd, J=8.6, 0.5 Hz, 1H).

### 2-(5-chlorobenzo[b]thiophen-3-ylmethyl)-3-hydroxy-7,8,9,10-tetrahydrobenzo[h]quinoline-4-carboxylic acid (Compound 15)

The procedure described above for the synthesis and purification of compound 7 was followed, reacting 6,7,8,9-tetrahydrobenzo[g]indoline-2,3-dione (0.200 g, 0.994 mmol) with 3-(5-chlorobenzo[b]thiophen-3-yl)-2-oxopropyl acetate (0.365 g, 1.29 mmol). It was not possible to convert the triethylammonium salt obtained by preparative HPLC (basic modifier) back to the free acid by the usual method. Thus, the final product, a sunflower-yellow powder, was a triethylammonium salt with 6:5 acid:base stoichiometry (108 mg, 21% yield): ¹H NMR (400 MHz, DMSO-d₆) δ 1.17 (t, J=7.2 Hz, 7.5H) 1.72-1.87 (m, 4H) 2.77 (t, J=5.9 Hz, 2H) 3.10 (dq, 5H) 3.18 (t, J=5.7 Hz, 2H) 4.46 (s, 2H) 7.08 (d, J=8.8 Hz, 1H) 7.35 (dd, J=8.7, 2.2 Hz, 1H) 7.59 (s, 1H) 7.96 (d, J=8.3 Hz, 1H) 8.41 (d, J=2.1 Hz, 1H) 8.94 (d, J=8.8 Hz, 1H); HRMS (ESI+) calcd for C₂₃H₁₉ClNO₃S (MH+) 424.0769, found 424.0770. Anal. Calcd for [C₂₃H₁₉ClNO₃S]₆[C₆H₁₅N]₅[H₂O]: C, 65.60; H, 5.78; N, 4.72. Found: C, 64.75; H, 6.01; N, 4.56.

### Example 16

### Preparation of 3-hydroxy-2-phenyl-7,8,9,10-tetrahydro-benzo[h]quinoline-4-carboxylic acid (compound 16)

### Compound 16 was Prepared According to Scheme 16 Below:

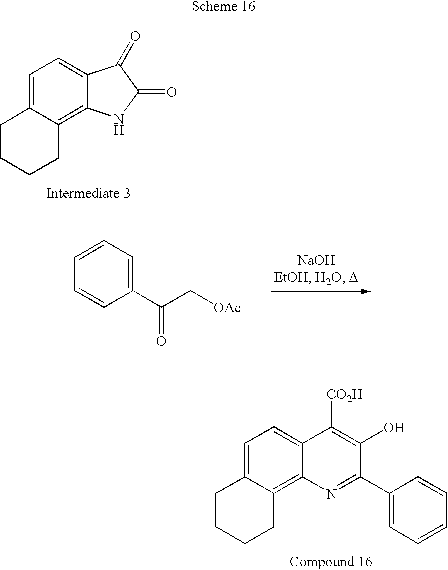

### 3-hydroxy-2-phenyl-7,8,9,10-tetrahydro-benzo[h]quinoline-4-carboxylic acid (compound 16)

The procedure described above for the synthesis and purification of compound 7 was followed, reacting 6,7,8,9-tetrahydrobenzo[g]indoline-2,3-dione (0.294 g, 1.46 mmol) with phenacyl acetate (0.338 g, 1.90 mmol). Product was obtained as a yellow powder (116 mg, 25% yield): ¹H NMR (400 MHz, DMSO-D6) δ ppm 1.75-1.93 (m, 4H) 2.86 (t, J=5.68 Hz, 2H) 3.25 (t, J=5.81 Hz, 2H) 7.33 (d, J=9.09 Hz, 1H) 7.44-7.56 (m, 3H) 8.09 (dd, J=8.08, 1.52 Hz, 2H) 8.28 (d, J=8.84 Hz, 1H).

### Example 17

### Preparation of 2-(4-cyano-benzyl)-3-hydroxy-7,8,9,10-tetrahydro-benzo[h]quinoline-4-carboxylic acid (Compound 17)

### Compound 17 was Prepared According to Scheme 17 Below:

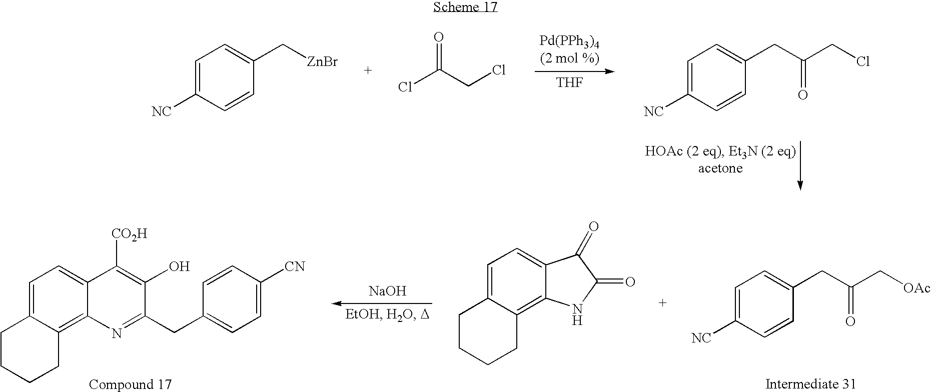

### Intermediate 31: acetic acid 3-(4-cyano-phenyl)-2-oxo-propyl ester

The procedure described above for the synthesis of 3-(3-chlorophenyl)-2-oxopropyl acetate was followed, reacting 0.5 M THF solution of 4-cyanobenzylzinc bromide (26 mL, 13 mmol), Pd(PPh₃)₄ (0.30 g, 0.26 mmol) with chloroacetyl chloride (26 mL, 13 mmol). Work-up of the reaction mixture gave crude product as a yellow oil.

This crude material was reacted with acetic acid (1.42 mL, 1.49 g, 24.8 mmol) and triethylamine (3.46 mL, 2.51 g, 24.8 mmol), as described above for the synthesis of 3-(3,4-dichlorophenyl)propan-2-one. Flash chromatography over silica gel (10-30% ethyl acetate in hexanes) gave pure product (0.71 g, 25% yield). ¹H NMR (400 MHz, DMSO-D6) 6 ppm 2.09 (s, 3H) 3.96 (s, 2H) 4.88 (s, 2H) 7.40 (d, J=8.34 Hz, 2H) 7.79 (d, J=8.59 Hz, 2H)

### 2-(4-cyano-benzyl)-3-hydroxy-7,8,9,10-tetrahydro-benzo[h]quinoline-4-carboxylic acid (Compound 17)

In a 25 mL round-bottomed flask, 6,7,8,9-tetrahydrobenzo[g]indoline-2,3-dione (0.119 g, 0.590 mmol) was taken up in 1 mL ethanol and 3 mL 10 M NaOH, and the mixture heated at reflux temperature for 3 minutes. A solution of acetic acid 3-(4-cyano-phenyl)-2-oxo-propyl ester (0.167 g, 0.767 mmol) in 3 mL ethanol was then added and the reaction further heated for 10 minutes. The reaction mixture was then cooled to room temperature and acidified with glacial acetic acid, and the yellow precipitate collected by filtration. The procedure described above for the purification of example 7 was followed. Product was obtained as a bright yellow powder (42 mg, 20% yield): ¹H NMR (400 MHz, DMSO-D6) δ ppm 1.74-1.87 (m, 4H) 2.83 (t, J=5.31 Hz, 2H) 3.12 (t, J=5.43 Hz, 2H), 4.40 (s, 2H), 7.28 (d, J=9.09 Hz, 1H) 7.51 (d, J=8.59 Hz, 2H) 7.75 (d, J=8.34 Hz, 2H) 8.23 (d, J=8.84 Hz, 1H).

### Examples 18 and 19

### Preparation of 2-(4-carboxy-benzyl)-3-hydroxy-7,8,9,10-tetrahydro-benzo[h]quinoline-4-carboxylic acid (Compound 18) and 2-(4-carbamoyl-benzyl)-3-hydroxy-7,8,9,10-tetrahydro-benzo[h]quinoline-4-carboxylic acid (Compound 19)

Compounds 18 and 19 were Prepared According to Scheme 18 Below:

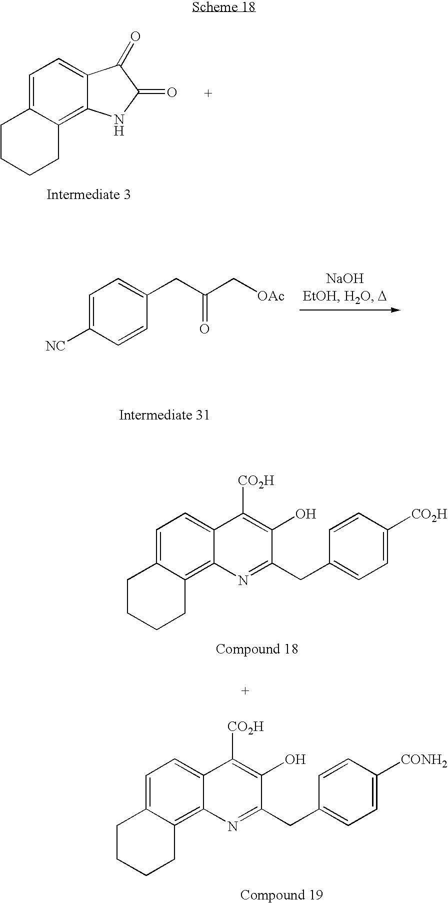

### 2-(4-carboxy-benzyl)-3-hydroxy-7,8,9,10-tetrahydro-benzo[h]quinoline-4-carboxylic acid (Compound 18) and 2-(4-carbamoyl-benzyl)-3-hydroxy-7,8,9,10-tetrahydro-benzo[h]quinoline-4-carboxylic acid (Compound 19)

The procedure described above for the synthesis and purification of compound 7 was followed, reacting 6,7,8,9-tetrahydrobenzo[g]indoline-2,3-dione (0.495 g, 2.46 mmol) with acetic acid 3-(4-cyano-phenyl)-2-oxo-propyl ester (0.694 g, 3.20 mmol). Two products were isolated as bright yellow powders. Compound 18 was obtained in 15% yield (139 mg): ¹H NMR (400 MHz, DMSO-D6) δ ppm 1.76-1.88 (m, 4H) 2.84 (t, J=6.69 Hz, 2H) 3.16 (t, J=6.32 Hz, 2H) 4.39 (s, 2H) 7.30 (d, J=8.84 Hz, 1H) 7.42 (d, J=8.59 Hz, 2H) 7.77 (d, J=8.34 Hz, 2H) 8.43 (d, J=8.84 Hz, 1H). Compound 19 was obtained in 10% yield (92 mg): ¹H NMR (400 MHz, DMSO-D6) δ ppm 1.77-1.89 (m, 4H) 2.84 (t, J=6.44 Hz, 2H) 3.16 (t, J=5.81 Hz, 2H) 4.39 (s, 2H) 7.30 (d, J=8.84 Hz, 1H) 7.42 (d, J=8.59 Hz, 2H) 7.77 (d, J=8.34 Hz, 2H) 8.43 (d, J=8.84 Hz, 1H).

### Example 20

### Preparation of 2-benzyl-3-hydroxy-7,8,9,10-tetrahydro-benzo[h]quinoline-4-carboxylic acid (Compound 20)

### Compound 20 was Prepared According to Scheme 19 Below:

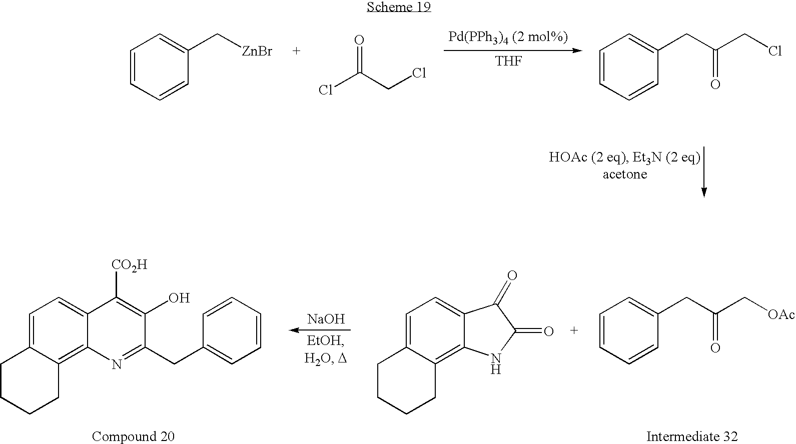

### Intermediate 32: acetic acid 2-oxo-3-phenyl-propyl ester

The procedure described above for the synthesis of 3-(3-chlorophenyl)-2-oxopropyl acetate was followed, reacting 0.5 M THF solution of benzylzinc bromide (26 mL, 13 mmol), Pd(PPh₃)₄ (0.30 g, 0.26 mmol) with chloroacetyl chloride (26 mL, 13 mmol). Work-up of the reaction mixture gave crude product as a yellow oil.

This crude material was reacted with acetic acid (1.42 mL, 1.49 g, 24.8 mmol) and triethylamine (3.46 mL, 2.51 g, 24.8 mmol), as described above for the synthesis of 3-(3,4-dichlorophenyl)propan-2-one. Flash chromatography over silica gel (10-30% ethyl acetate in hexanes) gave pure product (0.83 g, 33% yield). ¹H NMR (400 MHz, DMSO-D6) δ ppm 2.08 (s, 3H) 3.80 (s, 2H) 4.85 (s, 2H) 7.17-7.36 (m, 5H).

### 2-benzyl-3-hydroxy-7,8,9,10-tetrahydro-benzo[h]quinoline-4-carboxylic acid (Compound 20)

The procedure described above for the synthesis and purification of example 7 was followed, reacting 6,7,8,9-tetrahydrobenzo[g]indoline-2,3-dione (0.294 g, 1.46 mmol) with acetic acid 2-oxo-3-phenyl-propyl ester (0.364 g, 1.90 mmol). Product was obtained as a yellow powder (171 mg, 35% yield): ¹H NMR (400 MHz, DMSO-D6) δ ppm 1.75-1.89 (m, 4H) 2.83 (t, J=6.06 Hz, 2H) 3.17 (t, J=6.10 Hz, 2H) 4.31 (s, 2H) 7.13-7.21 (m, 1H) 7.23-7.36 (m, 5H) 8.24 (d, J=9.09 Hz, 1H).

### Example 21

### Preparation of 3-hydroxy-2-phenethyl-7,8,9,10-tetrahydro-benzo[h]quinoline-4-carboxylic acid (Compound 21)

### Compound 21 was Prepared According to Scheme 20 Below:

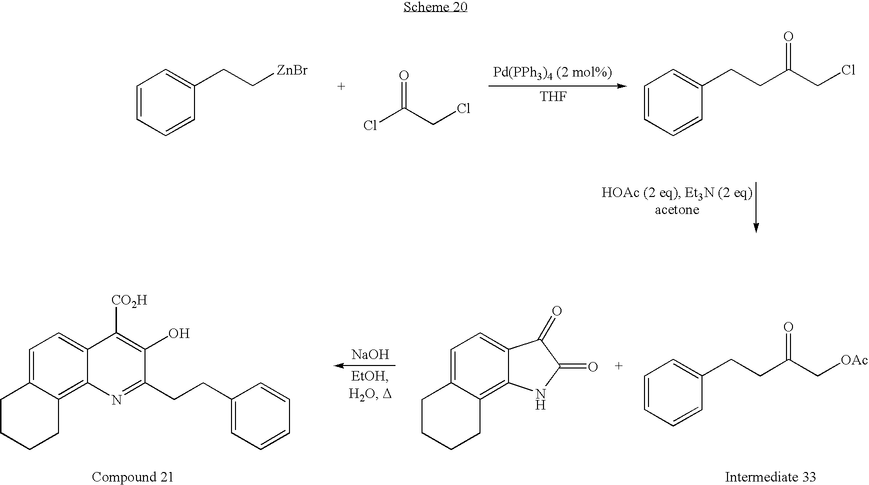

### Intermediate 33: Acetic acid 2-oxo-4-phenyl-butyl ester

The procedure described above for the synthesis of 3-(3-chlorophenyl)-2-oxopropyl acetate was followed, reacting 0.5 M THF solution of phenylethylzinc bromide (26 mL, 13 mmol), Pd(PPh₃)₄ (0.30 g, 0.26 mmol) with chloroacetyl chloride (26 mL, 13 mmol). Work-up of the reaction mixture gave crude product as a yellow oil.

This crude material was reacted with acetic acid (1.42 mL, 1.49 g, 24.8 mmol) and triethylamine (3.46 mL, 2.51 g, 24.8 mmol), as described above for the synthesis of 3-(3,4-dichlorophenyl)propan-2-one. Flash chromatography over silica gel (10-30% ethyl acetate in hexanes) gave an impure mixture, which was used as such for the next step.

### 3-hydroxy-2-phenethyl-7,8,9,10-tetrahydro-benzo[h]quinoline-4-carboxylic acid (Compound 21)

The procedure described above for the synthesis and purification of example 7 was followed, reacting 6,7,8,9-tetrahydrobenzo[g]indoline-2,3-dione (0.294 g, 1.46 mmol) with acetic acid 2-oxo-4-phenyl-butyl ester (0.391 g (75% purity), 1.90 mmol). Product was obtained as a yellow powder (76 mg, 15% yield): ¹H NMR (500 MHz, DMSO-D6) 6 ppm 1.76-1.91 (m, 4H) 2.85 (t, J=5.95 Hz, 2H) 3.16 (t, J=7.80 Hz, 2H) 3.22 (t, J=6.10 Hz, 2H) 3.29 (t, J=7.78 Hz, 2H) 7.18 (t, J=7.02 Hz, 1H) 7.23-7.35 (m, 5H) 8.27 (d, J=7.93 Hz, 1H).

### Example 22

### Preparation of 2-(4-chloro-benzyl)-3-hydroxy-7,8,9,10-tetrahydro-[1,9]phenanthroline-4-carboxylic acid (Compound 22)

### Compound 22 was Prepared According to Scheme 21 Below:

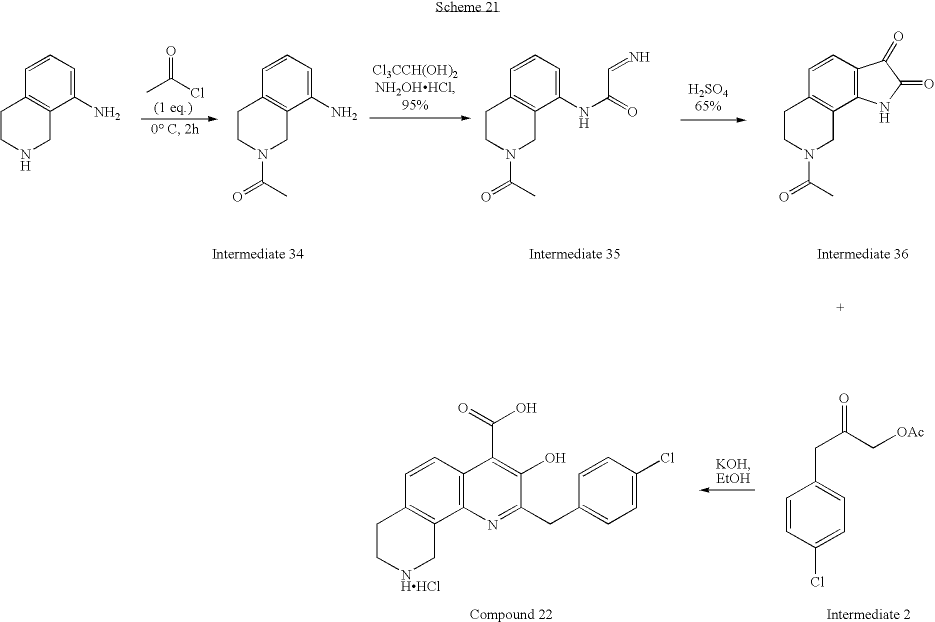

### Intermediate 34: 1-(8-amino-3,4-dihydro-1H-isoquinolin-2-yl)-ethanone (a mixture of two isomers in a 2:3 ratio)

To a solution of 1,2,3,4-tetrahydro 5-aminoisoquinoline (2.1 g, 14.1 mmol) in 125 mL dichloromethane and 100 mL saturated NaHCO₃ (aq.) at 0° C. was added acetyl chloride (1 mL, 14.1 mmol) in 25 mL dichloromethane dropwise. The resulting mixture was stirred at 0° C. for 30 minutes and the resulting organic layer was separated quickly so that the organic layer remained relatively cool. To the organic layer was immediately added methylamine hydrochloride (1 g, 14.2 mmol) and diisopropylamine (2 mL, 14.1 mmol) to scavenge the unreacted acetyl chloride. Removal of the solvent followed by flash chromatography (silica gel, ethyl acetate:hexane=5:1) gave the desired amide 34 as a light yellow oil (2 g, 74%). ¹H NMR (400 MHz, DMSO-D6) δ ppm 2.04 (s, 1.2H), 2.07 (s, 1.8H), 2.41 (dd, J=6.06, 6.19 Hz, 1H), 2.52 (m, 1H), 3.66 (dd, J=6.06, 6.19 Hz, 2H), 4.48 (s, 1.2H), 4.51 (s, 0.8H), 4.85-4.93 (bs, 2H), 6.36 (dd, J=7.33, 7.33 Hz, 1H), 6.47 (d, J=7.33 Hz, 0.6H), 6.49 (d, J=7.33 Hz, 0.4H), 6.85 (d, J=7.33 Hz, 0.6H) 6.88 (d, J=7.33 Hz, 0.4H).

### Intermediate 35: N-(2-acetyl-1,2,3,4-tetrahydro-isoquinolin-8-yl)-2-imino-acetamide (a mixture of two isomers in a 2:3 ratio)

Isatin synthesis described by Yang et al. (J. Am. Chem. Soc., 1996, 118, 9557) was used. A mixture of chloral hydrate (2.4 g, 14.9 mmol), hydroxylamine hydrochloride (3.3 g, 47.8 mmol), sodium sulfate (19 g, 133.8 mmol), intermediate 34 (2.4 g, 12.6 mmol), aq. HCl (10 mL, 1N), and 90 mL water was stirred at 55° C. overnight. The reaction mixture was cooled to 25° C. The precipitate was collected by filtration, washed with water, and dried under vacuum overnight to provide the intermediate 35 (2.8 g, 85%) as a beige solid which was used without further purification in the next step. ¹H NMR (400 MHz, DMSO-D6) δ ppm 2.07 (s, 1.8H), 2.08 (s, 1.2H), 2.62 (dd, J=5.94, 5.94 Hz, 0.8H), 2.72 (dd, J=5.94, 5.94 Hz, 1.2H), 3.63 (dd, J=6.06, 6.06 Hz, 2H), 4.61 (s, 1.2H), 4.66 (s, 0.8H), 7.07 (s, 0.4H), 7.09 (s, 0.6H), 7.19 (d, J=8.00 Hz, 0.4H), 7.21-7.25 (d, J=8.00 Hz, 0.6H), 7.30 (d, J=7.83 Hz, 0.4H) 7.33 (d, J=7.83 Hz, 0.6H), 7.66 (s, 1H), 9.61 (s, 1H), 12.19 (s, 1H).

### Intermediate 36: 8-acetyl-6,7,8,9-tetrahydro-1H-pyrrolo[3,2-h]isoquinoline-2,3-dione (a mixture of two isomers in a 2:3 ratio)

Intermediate 35 from above was mixed with 11 mL concentrated sulfuric acid at 25° C. The resulting dark purple solution was heated to 85° C. gradually and stayed at this temperature for 10 minutes. The reaction mixture was then cooled to 25° C. 50 mL crushed ice was added, and the reaction mixture was allowed to stay at 0° C. for 30 minutes. The precipitate was collected by filtration, washed with water, and dried under vacuum overnight to give isatin 36 (1.7 g, 65%) as an orange solid, which was used for the next step without further purification. ¹H NMR (400 MHz, DMSO-D6) δ ppm 2.08 (s, 1.2H), 2.10 (s, 1.8H), 2.58 (dd, J=5.81, 6.06 Hz, 0.8H), 2.69 (dd, J=5.81, 6.06 Hz, 1.2H), 3.70 (dd, J=6.23, 6.23 Hz, 2H), 4.63 (s, 1.2H), 4.69 (s, 0.8H), 6.91 (d, J=7.58 Hz, 0.4H), 6.92 (d, J=7.58 Hz, 0.6H), 7.33 (d, J=7.83 Hz, 0.4H), 7.37 (d, J=7.83 Hz, 0.6H), 11.12 (s, 0.4H), 11.15 (s, 0.6H).

### 2-(4-chloro-benzyl)-3-hydroxy-7,8,9,10-tetrahydro-[1,9]phenanthroline-4-carboxylic acid (Compound 22)

The procedure described by Cragoe et al. (J. Org. Chem., 1953, 18, 561) was used. To a mixture of isatin 36 (0.85 g, 3.48 mmol) in 2 mL EtOH and 4 mL aq. 6 M KOH at 100° C. was added warm 3-(4-chlorophenyl)-2-oxopropyl acetate (0.9 g, 3.98 mmol) in 2 mL EtOH in small portions over 1 hour period. After the addition was completed, the reaction mixture was refluxed for additional 1 h. Removal of the solvent, the resulting yellow gum was acidified with aq. 1 N HCl to pH˜-1. HPLC of the yellow precipitate under basic conditions afforded white solid, which was acidified at 0° C. with 1 N aq. HCl to pH˜1. The precipitate was collected by centrifuge, washed with water, and dried under vacuum to yield compound 22 (0.144 g, 16%) as a yellow solid. ¹H NMR (400 MHz, DMSO-D6) δ ppm 2.51-2.56 (m, 2H), 3.37-3.42 (m, 2H), 4.23 (s, 2H), 4.33 (bs, 2H), 7.18 (d, J=9.09 Hz, 1H), 7.27-7.33 (m, 2H), 7.33-7.39 (m, 2H), 8.95 (bs, 2H), 9.31 (d, J=9.09 Hz, 1H).

### Example 23

### Preparation of 2-(4-chloro-benzyl)-3-hydroxy-9-isopropyl-7,8,9,10-tetrahydro-[1,9]phenanthroline-4-carboxylic acid (Compound 23)

### Compound 23 was Prepared According to Scheme 22 Below:

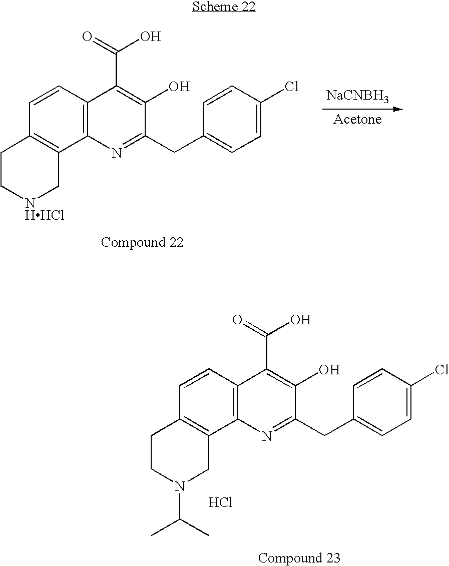

### 2-(4-chloro-benzyl)-3-hydroxy-9-isopropyl-7,8,9,10-tetrahydro-[1,9]phenanthroline-4-carboxylic acid (Compound 23)

A mixture of compound 22 (0.12 g, 0.297 mmol), triethylamine (46 μL, 0.30 mmol), acetone (26 μL, 0.446 mmol), sodium cyanoborohydride (23 mg, 0.36 mmol), 3 mL methanol, and 3 drops of acetic acid was stirred at 25° C. overnight. LC/MS showed that about half of the starting material left. Water and triethylamine were added dropwise to dissolve the precipitate. HPLC of the clear reaction mixture afforded a white solid, which was acidified with aq. 1 N HCl to pH˜1. The precipitate was collected by centrifuge, washed with water, and dried under vacuum to yield compound 23 (8.4 mg, 32% based on consumed starting material) as a white solid. ¹H NMR (400 MHz, DMSO-D6) δ ppm 1.43 (d, J=6.57, 1.77 Hz, 3H), 1.43 (d, J=6.57, 3H), 3.30-3.48 (m, 2H), 3.61-3.92 (m, 3H), 4.38-4.61 (m, 4H), 7.21-7.32 (m, 3H) 7.39 (d, J=8.34 Hz, 2H) 9.32 (d, J=9.09 Hz, 1H).

### Example 24

### Preparation of 9-benzyl-2-(4-chloro-benzyl)-3-hydroxy-7,8,9,10-tetrahydro-[1,9]phenanthroline-4-carboxylic acid (Compound 24)

### Compound 24 was Prepared According to Scheme 23 Below:

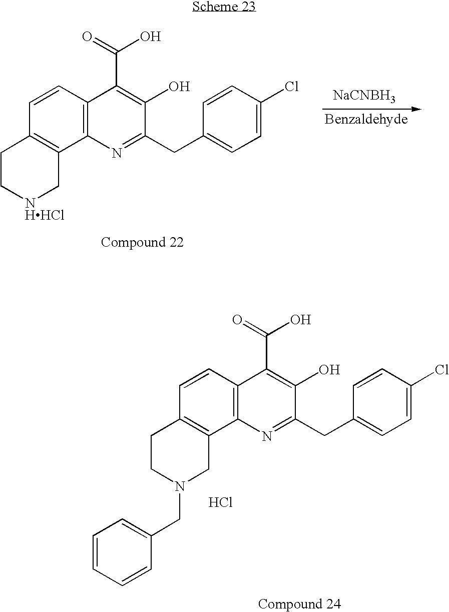

### 9-benzyl-2-(4-chloro-benzyl)-3-hydroxy-7,8,9,10-tetrahydro-[1,9]phenanthroline-4-carboxylic acid (Compound 24)

The procedure described above for the synthesis and purification of example 23 was followed, reacting 2-(4-chloro-benzyl)-3-hydroxy-7,8,9,10-tetrahydro-[1,9]phenanthroline-4-carboxylic acid (0.12 g, 0.297 mmol) with benzaldehyde to give compound 24 (24.1 mg, 40%). White solid. ¹H NMR (400 MHz, DMSO-D6) δ ppm 3.32-3.54 (m, 2H), 3.67-3.96 (m, 2H), 4.29 (s, 2H), 4.38-4.47 (m, 2H), 4.52 (s, 2H), 7.21 (d, J=8.84 Hz, 1H), 7.24-7.33 (m, 2H), 7.34-7.43 (m, 2H), 7.48-7.57 (m, 3H), 7.56-7.67 (m, 2H), 9.31 (d, J=8.84 Hz, 1H).

### Example 25

### Preparation of 2-(4-chloro-benzyl)-9-ethyl-3-hydroxy-7,8,9,10-tetrahydro-[1,9]phenanthroline-4-carboxylic acid (Compound 25)

### Compound 25 was Prepared According to Scheme 24 Below:

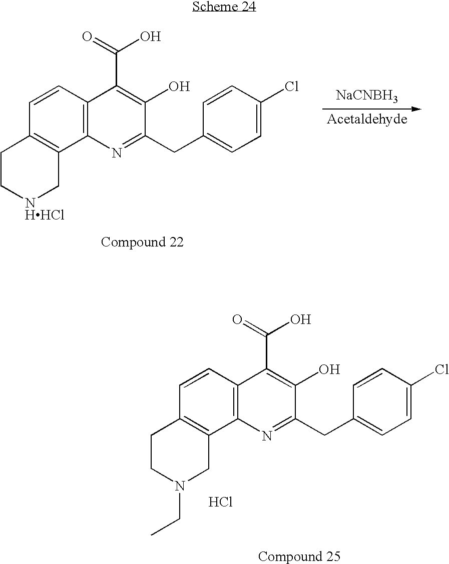

### 2-(4-chloro-benzyl)-9-ethyl-3-hydroxy-7,8,9,10-tetrahydro-[1,9]phenanthroline-4-carboxylic acid (Compound 25)

The procedure described above for the synthesis and purification of example 23 was followed, reacting 2-(4-chloro-benzyl)-3-hydroxy-7,8,9,10-tetrahydro-[1,9]phenanthroline-4-carboxylic acid (0.12 g, 0.297 mmol) with acetaldehyde to give compound 25 (2.2 mg, 3.4% based on consumed starting material). Light yellow solid. ¹H NMR (400 MHz, DMSO-D6) δ ppm 1.38 (t, J=7.33 Hz, 3H), 2.55-2.60 (m, 1H), 2.66-2.76 (m, 1H), 3.34 (q, J=7.33 Hz, 2H), 3.64-3.93 (m, 2H), 4.30 (s, 2H), 4.40 (d, J=15.16 Hz, 1H), 4.62 (d, J=15.16 Hz, 1H), 7.26-7.34 (m, 3H), 7.34-7.41 (m, 2H), 9.08 (d, J=8.08 Hz, 1H).

### Example 26

### Preparation of 9-acetyl-2-(4-chloro-benzyl)-3-hydroxy-7,8,9,10-tetrahydro-[1,9]phenanthroline-4-carboxylic acid (Compound 26) (mixture of two isomers)

### Compound 26 was Prepared According to Scheme 25 Below:

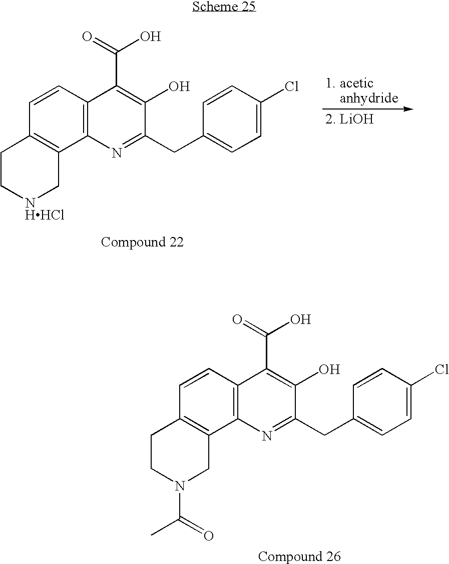

### 9-acetyl-2-(4-chloro-benzyl)-3-hydroxy-7,8,9,10-tetrahydro-[1,9]phenanthroline-4-carboxylic acid (Compound 26) (mixture of two isomers)

To 2-(4-chloro-benzyl)-3-hydroxy-7,8,9,10-tetrahydro-[1,9]phenanthroline-4-carboxylic acid (0.14 g, 0.346 mmol) in 2 mL pyridine was added triethylamine (60 μL, 0.43 mmol) and acetic anhydride (0.18 mL, 2.07 mmol) at 0° C. The reaction mixture was warmed to 25° C. and stirred overnight. HPLC of the reaction mixture afforded the acetamide ester (90 mg, 0.20 mmol) as a white solid, which was treated with LiOH (36 mg, 0.80 mmol) in 1 mL water. The mixture was stirred at 25° C. for 5 h. DMSO and triethylamine were added to the reaction mixture dropwise to dissolve the precipitate. HPLC of the clear solution gave compound 26 (20.7 mg, 25%) as a yellow solid. ¹H NMR (400 MHz, DMSO-D6) δ ppm 2.24 (s, 3H), 3.21-3.42 (m, 2H), 3.77-3.87 (m, 2H), 4.34 (s, 2H), 4.73-4.84 (m, 2H), 7.27-7.42 (m, 5H), 8.49-8.57 (m, 1H).

### Example 27

### Preparation of 9-carbamoyl-2-(4-chloro-benzyl)-3-hydroxy-7,8,9,10-tetrahydro-[1,9]phenanthroline-4-carboxylic acid (Compound 27)

### Compound 27 was Prepared According to Scheme 26 Below:

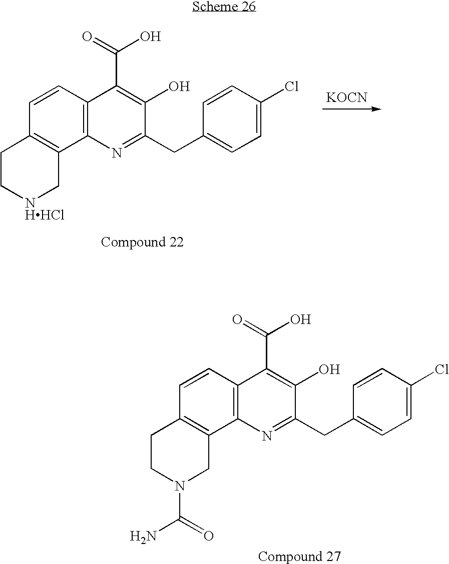

### 9-carbamoyl-2-(4-chloro-benzyl)-3-hydroxy-7,8,9,10-tetrahydro-[1,9]phenanthroline-4-carboxylic acid (Compound 27)

A mixture of 2-(4-chloro-benzyl)-3-hydroxy-7,8,9,10-tetrahydro-[1,9]phenanthroline-4-carboxylic acid (0.213 g, 0.53 mmol), acetic acid (0.6 mL, 5.3 mmol), triethylamine (0.146 mL, 1.06 mmol), KOCN (43 mg, 0.53 mmol), and pyridine (0.84 mL, 5.3 mmol) was stirred at 25° C. overnight. The solid was removed by filtration. HPLC of the mother liquor gave pure product (49.1 mg, 22%) as a beige solid. ¹H NMR (400 MHz, DMSO-D6) δ ppm 3.25 (m, 2H), 3.68 (m, 2H), 4.34 (s, 2H), 4.63 (s, 2H), 7.22-7.45 (m, 5H), 8.47 (d, J=9.09 Hz, 1H).

### Example 28 and 29

### Preparation of 9-benzoyl-2-(4-chloro-benzyl)-3-hydroxy-7,8,9,10-tetrahydro-[1,9]phenanthroline-4-carboxylic acid (Compound 28) and 9-benzoyl-3-benzoyloxy-2-(4-chloro-benzyl)-7,8,9,10-tetrahydro-[1,9]phenanthroline-4-carboxylic acid (Compound 29)

Compounds 28 and 29 were Prepared According to Scheme 27 Below:

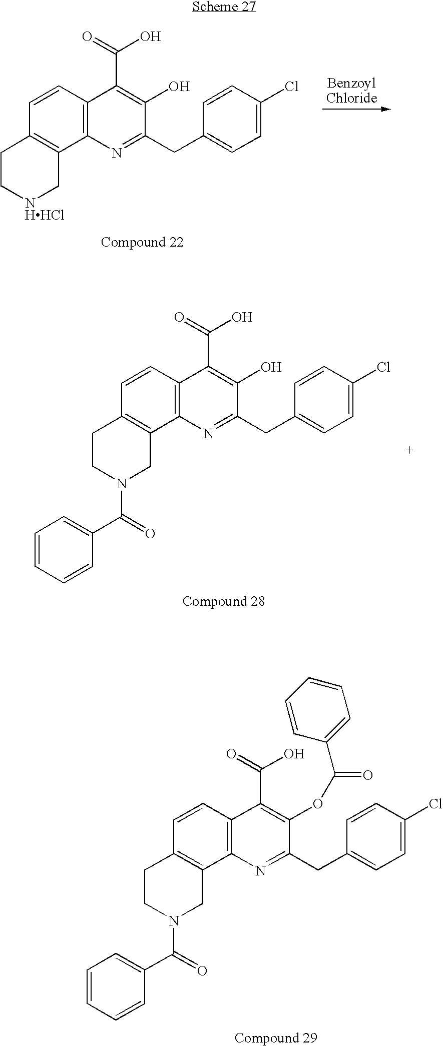

### 9-benzoyl-2-(4-chloro-benzyl)-3-hydroxy-7,8,9,10-tetrahydro-[1,9]phenanthroline-4-carboxylic acid (Compound 28) and 9-benzoyl-3-benzoyloxy-2-(4-chloro-benzyl)-7,8,9,10-tetrahydro-[1,9]phenanthroline-4-carboxylic acid (Compound 29)

To 2-(4-chloro-benzyl)-3-hydroxy-7,8,9,10-tetrahydro-[1,9]phenanthroline-4-carboxylic acid (0.132 g, 0.32 mmol) in 2 mL dichloromethane 0° C. was added benzoyl chloride (57 μL, 0.48 mmol) and triethylamine (0.10 mL, 0.74 mmol). The mixture was stirred at 25° C. overnight. HPLC of the mixture gave compound 28 (14.6 mg, 9.7%) as a yellow solid, and compound 29 (4.0 mg, 2.3%) as a white solid. Compound 28: ¹H NMR (500 MHz, DMSO-D6) δ ppm 3.32 (dd, J=5.80, 5.80 Hz, 2H), 3.81-3.83 (m, 2H), 4.34 (s, 2H), 4.81 (s, 2H), 7.27-7.34 (m, 3H), 7.35-7.41 (m, 2H), 7.43-7.54 (m, 5H), 8.52 (d, J=8.85 Hz, 1H).

Compound 29: ¹H NMR (400 MHz, DMSO-D6) δ ppm 3.37-3.46 (m, 2H), 3.56-3.60 (m, 2H), 4.28 (s, 2H), 5.00 (s, 2H), 7.15-7.34 (m, 4H), 7.45-7.57 (m, 6H), 7.64 (dd, J=7.71, 8.21 Hz, 2H), 7.80 (dd, J=7.71, 8.21 Hz, 1H), 7.88-7.98 (m, 1H), 8.10 (d, J=7.07 Hz, 2H).

### Example 30

### Preparation of 2-(4-chloro-benzyl)-3-hydroxy-9-methanesulfonyl-7,8,9,10-tetrahydro-[1,9]phenanthroline-4-carboxylic acid (Compound 30)

### Compound 30 was Prepared According to Scheme 28 Below:

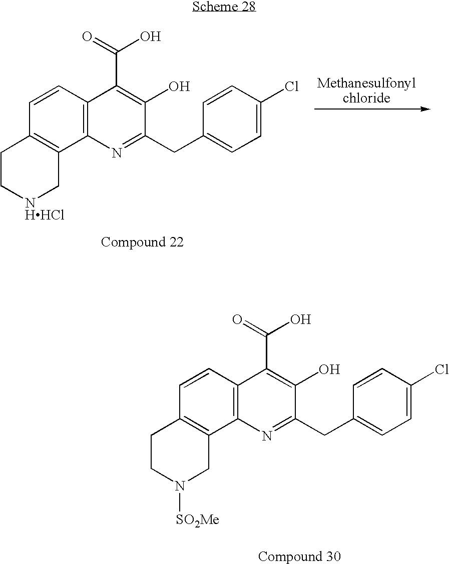

### 2-(4-chloro-benzyl)-3-hydroxy-9-methanesulfonyl-7,8,9,10-tetrahydro-[1,9]phenanthroline-4-carboxylic acid (Compound 30)

The procedure described above for the synthesis and purification of example 28 was followed, reacting 2-(4-chloro-benzyl)-3-hydroxy-7,8,9,10-tetrahydro-[1,9]phenanthroline-4-carboxylic acid (0.219 g, 0.54 mmol) with methanesulfonyl chloride (1 eq.) to give compound 30 (19 mg, 7.9%). ¹H NMR (400 MHz, DMSO-D6) δ ppm 2.97 (s, 3H), 3.34 (dd, J=5.68, 6.06 Hz, 2H), 3.53 (dd, J=5.68, 6.06 Hz, 2H), 4.27 (s, 2H), 4.45 (s, 2H), 7.25 (d, J=8.84 Hz, 1H), 7.31 (m, 2H), 7.37 (m, 2H), 8.97 (d, J=8.84 Hz, 1H).

### Example 31 and 32

### Preparation of 2-(4-chloro-benzyl)-3-hydroxy-7,10-dihydro-8H-[1,9]phenanthroline-4,9-dicarboxylic acid 9-ethyl ester (Compound 31) and 2-(4-chloro-benzyl)-3-ethoxycarbonyloxy-7,10-dihydro-8H-[1,9]phenanthroline-4,9-dicarboxylic acid 9-ethyl ester (compound 32)

Compounds 31 and 32 were Prepared According to Scheme 29 Below:

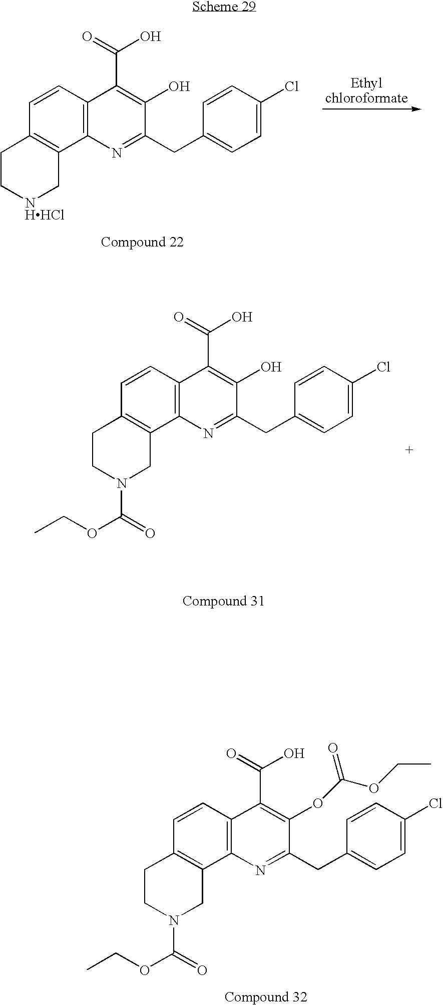

### 2-(4-chloro-benzyl)-3-hydroxy-7,10-dihydro-8H-[1,9]phenanthroline-4,9-dicarboxylic acid 9-ethyl ester (Compound 31) and 2-(4-chloro-benzyl)-3-ethoxycarbonyloxy-7,10-dihydro-8H-[1,9]phenanthroline-4,9-dicarboxylic acid 9-ethyl ester (compound 32)

The procedure described above for the synthesis and purification of example 28 was followed, reacting 2-(4-chloro-benzyl)-3-hydroxy-7,8,9,10-tetrahydro-[1,9]phenanthroline-4-carboxylic acid (0.13 g, 0.32 mmol) with ethyl chloroformate to give compound 31 (23.2 mg, 16.5%) as a yellow solid, and compound 32 (8.5 mg, 5.2%) as a white solid. Compound 31: ¹H NMR (400 MHz, DMSO-D6) δ ppm 1.24 (t, J=7.07 Hz, 3H), 3.25 (dd, J=5.68, 6.19 Hz, 2H), 3.73 (dd, J=5.68, 6.19 Hz, 2H), 4.12 (t, J=7.07 Hz, 2H), 4.32 (s, 2H), 4.67 (s, 2H), 7.30-7.42 (m, 5H), 8.37 (d, J=8.84 Hz, 1H). Compound 32: ¹H NMR (400 MHz, DMSO-D6) δ ppm 1.22 (t, J=7.16 Hz, 3H), 1.26 (t, J=7.07 Hz, 3H), 3.29 (dd, J=5.05, 5.81 Hz, 2H), 3.76 (dd, J=5.05, 5.81 Hz, 2H), 4.12 (q, J=7.16 Hz, 2H), 4.22 (q, J=7.07 Hz, 2H), 4.26 (s, 2H), 4.74 (s, 2H), 7.28 (m 2H), 7.35 (m, 2H), 7.54 (d, J=8.84 Hz, 1H), 7.85 (d, J=8.84 Hz, 1H).

### Example 33

### Preparation of 2-(4-chloro-benzyl)-3-hydroxy-9-phenylacetyl-7,8,9,10-tetrahydro-[1,9]phenanthroline-4-carboxylic acid (Compound 33) (mixture of two isomers)

### Compounds 33 was Prepared According to Scheme 30 Below:

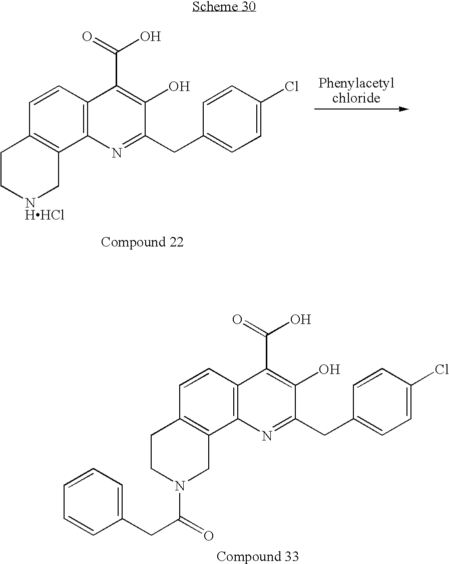

### Preparation of 2-(4-chloro-benzyl)-3-hydroxy-9-phenylacetyl-7,8,9,10-tetrahydro-[1,9]phenanthroline-4-carboxylic acid (Compound 33) (mixture of two isomers)

The procedure described above for the synthesis and purification of example 28 was followed, reacting 2-(4-chloro-benzyl)-3-hydroxy-7,8,9,10-tetrahydro-[1,9]phenanthroline-4-carboxylic acid (0.13 g, 0.32 mmol) with phenylacetyl chloride to give compound 33 (27.2 mg, 17.5%, mixture of two isomers in a 2:1 ratio) as a yellow solid. ¹H NMR (400 MHz, DMSO-D6) δ ppm 3.06-3.16 (m, 2H), 3.75-3.92 (m, 4H), 4.28 (s, 2H), 4.74 (s, 1.3H) 4.80-4.88 (m, 0.7H), 7.14-7.40 (m, 10H), 8.37-8.64 (m, 1H).

### Example 34

### Preparation of 2-(4-chloro-benzyl)-3-hydroxy-9-(propane-2-sulfonyl)-7,8,9,10-tetrahydro-[1,9]phenanthroline-4-carboxylic acid (Compound 34) (mixture of two isomers in a 2:1 ratio)

### Compounds 34 was Prepared According to Scheme 31 Below:

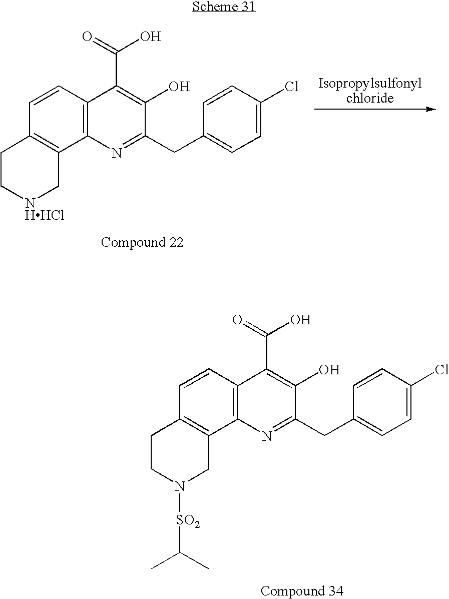

### Preparation of 2-(4-chloro-benzyl)-3-hydroxy-9-(propane-2-sulfonyl)-7,8,9,10-tetrahydro-[1,9]phenanthroline-4-carboxylic acid (Compound 34) (mixture of two isomers in a 2:1 ratio)

The procedure described above for the synthesis and purification of example 28 was followed, reacting 2-(4-chloro-benzyl)-3-hydroxy-7,8,9,10-tetrahydro-[1,9]phenanthroline-4-carboxylic acid (0.13 g, 0.32 mmol) with isopropylsulfonyl chloride (1 eq.) to give compound 34 as a yellow solid (5.2 mg, 3.4%, mixture of two isomers in a 2:1 ratio). ¹H NMR (500 MHz, DMSO-D6) δ ppm 1.23 (d, J=7.02 Hz, 6H), 3.11-3.14 (m, 2H), 3.23-3.32 (septlet, J=5.00 Hz, 1H), 3.56 (dd, J=5.95, 5.95 Hz, 0.6H), 3.63 (dd, J=5.95, 5.95 Hz, 1.4H), 4.25 (s, 2H), 4.46 (s, 0.6H), 4.53 (s, 1.4H), 7.23-7.27 (m, 1H), 7.28 (d, J=10.00 Hz, 2H) 7.33 (d, J=10.00 Hz, 2H) 8.78-8.87 (m, 1H).

### Example 35

### Preparation of 2-(4-chloro-benzyl)-3-methoxy-7,8,9,10-tetrahydro-benzo[h]quinoline-4-carboxylic acid (Compound 35)

### Compounds 35 was Prepared According to Scheme 32 Below:

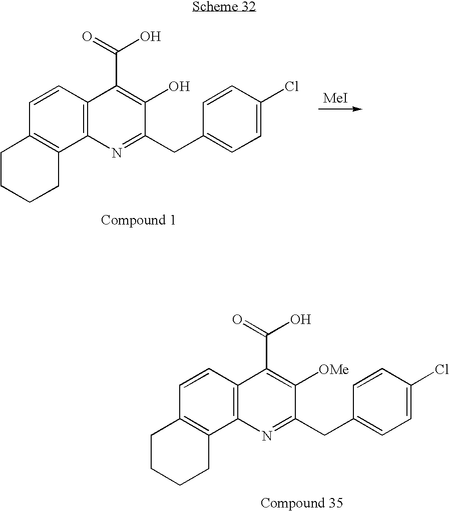

### Preparation of 2-(4-chloro-benzyl)-3-methoxy-7,8,9,10-tetrahydro-benzo[h]quinoline-4-carboxylic acid (Compound 35)

To 2-(4-chloro-benzyl)-3-hydroxy-7,8,9,10-tetrahydro-benzo[h]quinoline-4-carboxylic acid (0.117 g, 0.32 mmol) in 2 mL acetone at room temperature was added potassium carbonate (0.132 g, 0.96 mmol) and iodomethane (0.136 g, 0.96 mmol). The mixture was stirred overnight. HPLC of the mixture gave compound 35 (90 mg, 75%) as a white solid. ¹H NMR (400 MHz, DMSO-D6) δ ppm 1.69-1.94 (m, 4H), 2.76-2.88 (m, 2H), 3.11-3.19 (m, 2H), 3.80 (s, 3H), 4.21 (s, 2H), 7.15 (d, J=8.59 Hz, 1H), 7.31 (s, 4H), 7.49 (d, J=8.59 Hz, 1H).

### Examples 36 and 37

### Preparation of 3-hydroxy-2-piperidin-4-yl-7,8,9,10-tetrahydro-benzo[h]quinoline-4-carboxylic acid (Compound 36) and 2-(1-acetyl-piperidin-4-yl)-3-hydroxy-7,8,9,10-tetrahydro-benzo[h]quinoline-4-carboxylic acid (Compound 37)

Compounds 36 and 37 were Prepared According to Scheme 33 Below:

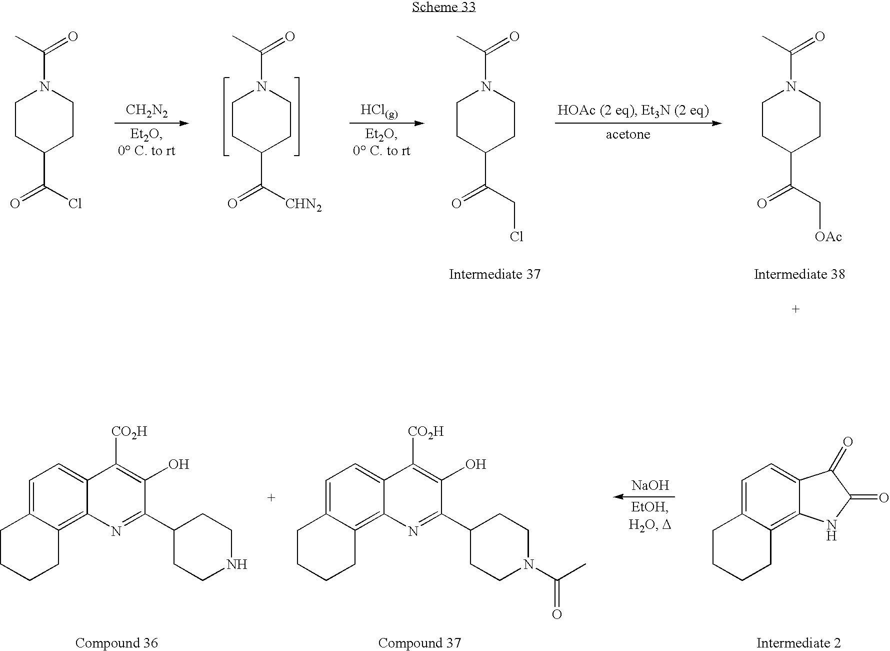

### 3-Hydroxy-2-piperidin-4-yl-7,8,9,10-tetrahydro-benzo[h]quinoline-4-carboxylic acid (Compound 36) and 2-(1-acetyl-piperidin-4-yl)-3-hydroxy-7,8,9,10-tetrahydro-benzo[h]quinoline-4-carboxylic acid (Compound 37)

Intermediate 37 was synthesized by Arndt-Eistert homologation of the acid chloride using the procedure described for 1-chloro-3-(thiophen-2-yl)propan-2-one (intermediate 15). The acid chloride (1.35 g, 7.1 mmol) was reacted with 40 mL of an ethereal diazomethane solution followed by passing HCl gas. The crude material was used as such in the next step. Synthesis of intermediate 38 was done using the procedure described above for the synthesis of 3-(3,4-dichlorophenyl)-2-oxopropyl acetate was followed, reacting 1-chloro-3-(thiophen-2-yl)propan-2-one (1.16 g, 5.73 mmol) with acetic acid (0.66 mL, 0.69 g, 12 mmol) and triethylamine (1.60 mL, 1.16 g, 11.5 mmol). The crude intermediate 40 was used as such in the next step.

Compounds 36 and 37 were synthesized using the procedure described above for the synthesis and purification of example 7, reacting 6,7,8,9-tetrahydrobenzo[g]indoline-2,3-dione (0.112 g, 0.557 mmol) with acetic acid 2-(1-acetyl-piperidin-4-yl)-2-oxo-ethyl ester (intermediate 40, 0.165 g, 0.724 mmol). Two products were isolated as white solids. Compound 36 (18.1 mg, 10% yield): ¹H NMR (400 MHz, DMSO-D6) δ ppm 1.72-1.89 (m, 4H) 1.96-2.19 (m, 4H) 2.69-2.87 (m, 2H) 3.06-3.16 (m, 2H) 3.19 (t, J=5.81 Hz, 2H) 3.38-3.50 (m, 2H) 3.52-3.67 (m, 1H) 7.07 (d, J=8.84 Hz, 1H) 8.28 (br s, 1H) 8.54 (br s, 1H) 9.17 (d, J=8.59 Hz, 1H); Compound 37 (10 mg, 5% yield): ¹H NMR (500 MHz, DMSO-D6) δ ppm 1.65-1.73 (m, 1H) 1.77-1.99 (m, 7H) 2.06 (s, 3H) 2.77 (t, J=11.44 Hz, 1H) 2.84 (t, J=6.10 Hz, 2H) 3.15-3.30 (m, 3H) 3.54 (t, J=11.14 Hz, 1H) 3.98 (d, J=13.73 Hz, 1H) 4.51 (d, J=13.73 Hz, 1H) 7.26 (d, J=8.85 Hz, 1H) 8.31 (d, J=8.85 Hz, 1H).

### Example 38

### Assay of Compounds of the Present Invention

Compounds of the present teachings can be assayed for selectin inhibitory activity using any of the procedures known in the art. One convenient procedure is the determination of IC50 values for inhibition of P-selectin binding to P-selectin glycoprotein ligand-1 (PSGL-1) using a Biacore instrument.

The Biacore 3000 is an instrument that uses surface plasmon resonance to detect binding of a solution phase analyte to an immobilized ligand on a sensor chip surface. The analyte sample is injected under flow using a microfluidic system. Binding of analyte to ligand causes a change in the angle of refracted light at the surface of the sensor chip, measured by the Biacore instrument in resonance units (RUs).

SGP-3 is a purified sulfoglycopeptide form of human PSGL-1 that contains the P-selectin binding determinants (See Somers et al., 2000, Cell 103, 467-479). SGP-3 was biotinylated via amine chemistry at a unique C-terminal lysine residue and immobilized on streptavidin-coated SA sensor chip. A solution containing a soluble recombinant truncated form of human P-selectin comprised of the lectin and EGF domains (P-LE) was delivered to the SGP-3 coated sensor chip. The P-LE solution contains 100 mM HEPES, 150 mM NaCl, 1 mM CaCl₂, 1 mM MgCl₂, 0.05% P40, 10% DMSO. K_{D} values were typically calculated to be approximately 778+/−105 nM using this Biacore assay format (Somers et al., supra).

Small molecule P-selectin inhibitors are incubated for 1 hour in 100 mM HEPES, 150 mM NaCl, 1 mM CaCl₂, 1 mM MgCl₂, 0.05% P40, 10% DMSO, prior to introducing them into the Biacore 3000. Solutions are filtered if formation of precipitate is visible. Soluble P-LE is added to the small molecule solution at final concentrations 500 nM and 500 μM respectively. Sample injections are run in duplicates, and each compound is assayed at least twice.

The Biacore assay measures the signal in RU produced by binding of P-LE to SGP-3 in the presence and absence of inhibitors. Percent inhibition of binding is calculated by dividing the inhibited signal by the uninhibited signal subtracting this value from one then multiplying by one hundred. Inhibitors, with greater than 50% inhibition at 500 μM, are assayed again using a series of two fold dilutions. The data from this titration are plotted, RU values vs. concentration, and the IC50 is determined by extrapolation from the plot. All RU values are blank and reference subtracted prior to percent inhibition and IC50 determination. Glycyrrhizin is used as a positive control, inhibiting 50% at 1 mM.

Compounds 1-6 were assayed as described above. IC50 values for four of the compounds ranged from 125 μM to 500 μM. One compound showed 17% inhibition at 500 μM, and one compound showed 11% inhibition at 125 μM.

Compounds 7-10, 17-20 and 22-33 also were tested as above. Six of the compounds displayed IC50 values ranging from 100 μM to 1250 μM. The percentage inhibition at 250 μM for an additional three compounds ranged from 46% to 58%. The percentage inhibition at 500 μM for an additional ten compounds ranged from 5% to 55%, with three of the compound showing no significant percentage inhibition at that concentration. One further compound displayed 24% inhibition at 1000 μM.

### Examples 39 and 40

### Effect of 2-(4-chlorobenzyl)-3-hydroxy-7,8,9,10-tetrahydrobenzo[h]quinoline-4-carboxylic acid on leukocyte rolling in C57 black/6J mice and Sprague-Dawley Rats

In vivo confocal microscopy has been successfully used to visualize intra vascular leukocyte rolling and extravasation in conjunctival venules of patients. This non-invasive, real time technology permits observation, quantitation and monitoring of responses to therapeutic interventions by visualizing changes in conjunctival intra-vascular events. The procedure has been used in clinical trials to monitor leukocyte rolling and extravasation in: 1) patients with surgically-induced inflammation associated with senile cataract correction and response to pretreatment with hydrocortisone; (Kirveskari J., Hydrocortisone reduced in vivo, inflammation-induced slow rolling of leukocytes and their extravasation into human conjunctiva. [Blood. Sep. 15, 2002; 100(6): 2203-7), 2) patients with allergen-induced conjunctivitis and response to treatment with heparin (Helintö M, Direct in vivo monitoring of acute allergic reactions in human conjunctiva. J. Immunol. Mar. 1, 2004 172(5): 3235-42), 3) contact lens wearers (Nguyen T. H., Increased Conjunctival or Episcleral Leukocyte Adhesio in Patients who Wear Contact Lenses with Lower Oxygen Permeability (Dk) Values. Clinical Sciences 2004. 23(7):695-700). This technique can also be used to visualize changes in conjunctival microcirculation in patients with sickle cell disease in relation to disease flare-ups (Cheung A. T. W., Microvascular abnormalities in sickle cell disease: a computer-assisted intravital microscopy study. Blood. May 2002; 99(110:3999-4005). More recently, Dr. Rosenbaum at the Casey Eye Institute in Oregon has demonstrated increased leukocyte rolling in conjunctival vessels of patients with scleritis (unpublished work).

The effect of the P-Selectin inhibitor 2-(4-chlorobenzyl)-3-hydroxy-7,8,9,10-tetrahydrobenzo[h]quinoline-4-carboxylic acid (Compound 1) on leukocyte rolling was determined in mice and rats using intravital microscopy. Surgical exteriorization of cremasteric postcapillary venules induces P-selectin dependent rolling of leukocytes on the vascular endothelium. Upon exposure to inflammatory mediators, P-selectin is rapidly translocated to the surface of endothelial cells where it then binds to P-selectin glycoprotein ligand-1 (PSGL-1) that is constitutively expressed on circulating leukocytes. This binding interaction results in leukocytes capturing on the intravascular endothelial cell surface and initiation of leukocyte rolling on this inflamed surface. To assess the efficacy of the novel P-selectin antagonist Compound 1, we applied the method of intravital microscopy on a surgically traumatized cremaster muscle tissue. This method is particularly useful, because it allows real time assessment of the anti-inflammatory efficacy of P-selectin inhibitors by looking at changes in rolling flux of leukocytes in cremasteric postcapillary venules in vivo.

### Example 39

### Activity of Compound 1 in male C57 Black/6J wild type mice

The effect of Compound 1 on leukocyte rolling in male C57 Black/6J wild type mice, 8-12 weeks old, weighing from about 20 g to about 26 g, was determined using intravital microscopy.

### Materials:

2-(4-chlorobenzyl)-3-hydroxy-7,8,9,10-tetrahydrobenzo[h]quinoline-4-carboxylic acid (Compound 1) was suspended in a 2% Tween 80 (w/v) and 0.5% methylcellulose (w/v) mixture in water, prepared from 5% Tween 80 stock solution (J. T. Baker) and 2% methylcellulose stock solution (Fluka Chemical), yielding final concentrations of 1.25 mg/ml and 6.25 mg/ml, and dosed via oral gavage in a volume of 200 μl. The compound vehicle was a 2% Tween 80 and 0.5% methylcellulose mixture in water, prepared from 5% Tween 80 stock solution (J. T. Baker) and 2% methylcellulose stock solution (Fluka Chemical) and was dosed via oral gavage in a volume of 200 μl. Purified anti-mouse CD62P(P-selectin) antibody, clone RB40.34 (BD Pharmingen) was dosed by IV tail vein injection in a volume of 200 μl. Antibody vehicle was a 0.9% sodium chloride solution (Baxter International). Bicarbonate saline buffer, pH 7.4, used to superfuse the cremaster tissue, consisted of 132 mM NaCl (Fisher Scientific), 5 mM KCL (Fisher Scientific), 2 mM CaCl₂ (Fisher Scientific), 1 mM MgSO₄ (Fisher Scientific), 20 mM NaHCO₃ (EMD Biosciences).

### Dosing of Animals:

The mice were divided into four treatment groups. Three groups of six mice each, were dosed with vehicle, 10 mg/kg or 50 mg/kg of 2-(4-chlorobenzyl)-3-hydroxy-7,8,9,10-tetrahydrobenzo[h]quinoline-4-carboxylic acid (Compound 1) in 2% Tween 80 and 0.5% methylcellulose vehicle, in a volume 200 μl/mouse, by oral gavage. The fourth group of 2 mice received tail vein injections of anti-mouse CD62P antibody at 50 mg/mouse, in a volume of 200 μl. All dosing procedures and injections were performed 20 minutes prior to surgery and 35 minutes prior to recording data. After dosing, mice were anesthetized with an intraperitoneal injection of ketamine hydrochloride (150 mg/kg) and xylazine (7.5 mg/kg).

### Surgical Procedure:

The cremaster muscle was surgically exteriorized, place over an optically clear viewing window and superfused continuously with bicarbonate buffered-saline (pH 7.4) at 35° C. The cremaster muscle was observed through an intravital microscope (Zeiss Model: Axioscope FS, Thronwood, N.Y.) with a 40× magnification (NA 0.75) water immersion objective lenses and a 10× magnification eyepiece. The image of the postcapillary venule was illuminated using brightfield microscopy and recorded with a video camera (Panasonic Model: GP-KR222, Secaucus, N.J.) and videocassette recorder (Sony Model: SVT-S3100, Montvale, N.J.) for off-line analysis of leukocyte rolling.

### Quantitation of Rolling Cells:

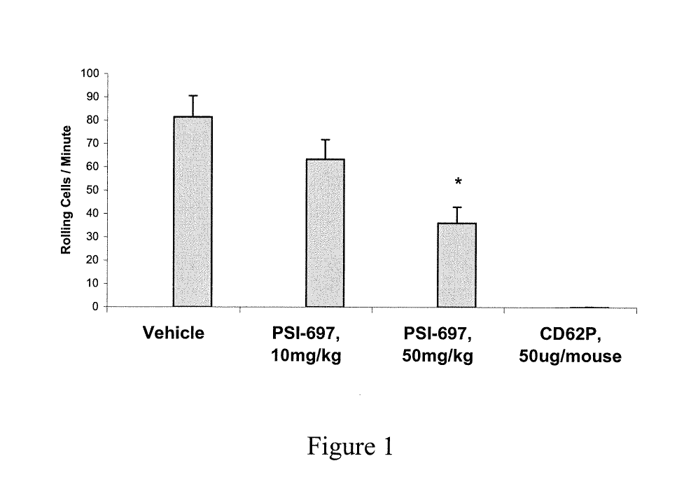

Ten postcapillary venules (20-45 mm diameter) of the cremaster muscle of each mouse were selected for observation. The number of rolling leukocytes was determined off-line during playback of videotaped images. Rolling Leukocytes are defined as leukocytes that moved at a velocity less than that of red blood cells in a given vessel and evaluated using frame-by-frame analysis. The number of rolling leukocytes (flux) was determined by counting all visible cells passing through a plane perpendicular to the vessel axis during one minute. The results are shown in FIG. 1.

### Statistical Analysis:

Data were expressed as Mean Standard Error of the Mean (SEM). All parameters of interest were subjected to Student T-test (for comparison between two groups) or Analysis of Variance with a Tukey post hoc testing between groups (for comparison between three groups or more) using GraphPad Prism software. Differences were considered significant if P<0.05.

### Results:

As can be seen in FIG. 1, a single oral dose of Compound 1 decreased rolling flux of leukocytes in postcapillary venules of compound-treated animals in a dose dependent manner (10 mg/kg −22%, 50 mg/kg −56% reduction) in comparison with vehicle-treated animals. Anti-mouse CD62P antibody completely ablated rolling, demonstrating the P-selectin dependence of the model (this monoclonal antibody binds to mouse P-selectin (CD62P) on inflammed endothelial cells blocking binding of the ligand PSGL-1 on leukocytes, preventing their recruitment).

### Example 40

### Effect of Compound 1 in male Sprague-Dawley Outbred Rats

The effect of Compound 1 on leukocyte rolling in male Sprague-Dawley outbred rats, 4-5 weeks old, weighing from about 50 g to about 100 g, was determined using intravital microscopy.

### Materials:

2-(4-chlorobenzyl)-3-hydroxy-7,8,9,10-tetrahydrobenzo[h]quinoline-4-carboxylic acid (Compound 1) was suspended in a 2% Tween 80 (w/v) and 0.5% methylcellulose (w/v) mixture in water, prepared from 5% Tween 80 stock solution (J. T. Baker) and 2% methylcellulose stock solution (Fluka Chemical), yielding final concentrations of 1.25 mg/ml and 6.25 mg/ml, and dosed via oral gavages in a volume of 200 μl/50 g body weight. The compound vehicle was a 2% Tween 80 and 0.5% methylcellulose mixture in water, prepared from 5% Tween 80 stock solution (J. T. Baker) and 2% methylcellulose stock solution (Fluka Chemical) and was dosed via oral gavage in a volume of 200 μl/50 g body weight. Purified anti-rat PSGL-1 antibody, produced at Wyeth, Cambridge, Mass., was dosed 4 mg/kg by IV jugular vein injection in a volume of 200 μl. Antibody vehicle was a 0.9% sodium chloride solution (Baxter International). Bicarbonate saline buffer, pH 7.4, used to superfuse the cremaster tissues, consisted of 132 mM NaCl (Baker Chemical), 5 mM KCl (Fisher Scientific), 2 mM CaCl₂ (EMD biosciences), 1 mM MgSO₄, (Fisher Scientific), 20 mM NaHCO₃ (EMD biosciences).

### Dosing of Animals:

The rats were divided into four treatment groups. One group of nine rats and two groups of six rats each, were dosed with vehicle, 30 mg/kg or 50 mg/kg of 2-(4-chlorobenzyl)-3-hydroxy-7,8,9,10-tetrahydrobenzo[h]quinoline-4-carboxylic acid (Compound 1) in 2% Tween 80 and 0.5% methylcellulose vehicle, in a volume 200 μl/mouse, by oral gavage, respectively. A fourth group of three rats received I.V. injections (through jugular vein canulation) of anti-rat PSGL-1 antibody at 4 mg/kg, in a volume of 200 μl.

All dosing procedures and injections were performed 20 minutes prior to surgery and 35 minutes prior to recording data. After dosing, rats were anesthetized with an intramuscular injection of ketamine hydrochloride (80 mg/kg) and xylazine (10 mg/kg).

The surgical procedure, quantitation of rolling cells and statistical analyses were performed as described in Example 39.

### Results:

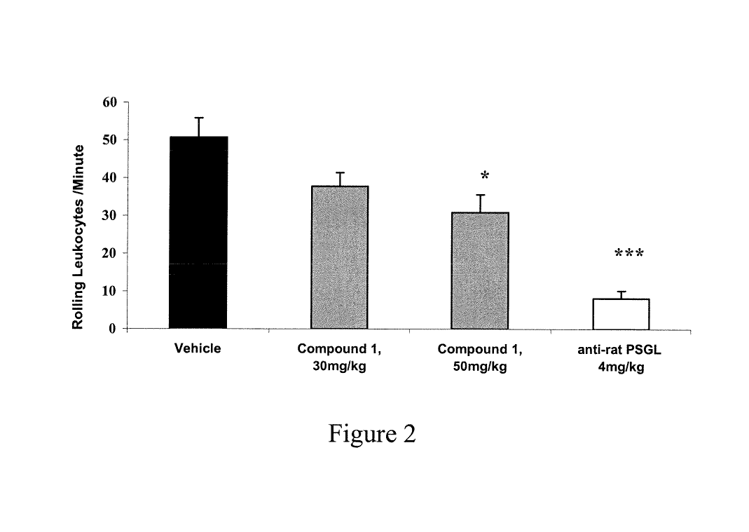

As can be seen in FIG. 2, a single oral dose of 2-(4-chlorobenzyl)-3-hydroxy-7,8,9,10-tetrahydrobenzo[h]quinoline-4-carboxylic acid (Compound 1) decreased rolling flux of leukocytes in postcapillary venules of compound-treated animals in dose dependent manner (30 mg/kg −25%, 50 mg/kg −39% reduction) in comparison with vehicle-treated animals. The anti-rat PSGL-1 antibody almost completely ablated rolling (84%), demonstrating the P-selectin dependence of the model (it is known that PSGL-1, a ligand for P-selectin, is constitutively expressed on all leukocytes and bonds between P-selectin and PSGL-1 primarily mediate the rolling phase of the adhesion cascade).

Variations, modifications, and other implementations of what is described herein will occur to those of ordinary skill in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced herein.

This application claims the benefit of U.S. Provisional Application No. 60/849,580, filed Oct. 5, 2006, the entire disclosure of which is incorporated by reference herein.

Each reference cited in the present application, including books, patents, published applications, journal articles and other publications, is incorporated herein by reference in its entirety.

What is claimed is:

1. A method of treating scleritis, a scleritis symptom or a scleritis-related condition, the method comprising administering to a subject a therapeutically effective amount of a compound having the Formula I: 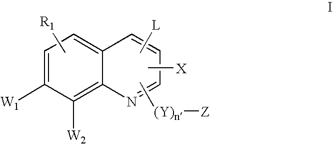
wherein:
W₁ and W₂ taken together with the atoms to which they are attached form a 5 or 6 member carbocyclic or heterocyclic ring that can be saturated, partially saturated or aromatic, and that can be substituted with up to three groups selected from the group consisting of hydrogen, C₁₋₆ alkyl, C₁₋₆ perhaloalkyl, OC₁₋₆ alkyl, OC₁₋₆ perhaloalkyl, halogen, thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, C₆₋₁₄ aryl, 3 to 14 membered heterocyclo, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄ aryl, 3 to 14 membered Oheterocyclo, C₇₋₂₄ arylalkyl, C(═O)C₇₋₂₄arylalkyl, OC(═O)C₇₋₂₄ arylalkyl, OC₇₋₂₄ arylalkyl, C₂₋₂₀ alkenyl, C₂₋₂₀ alkynyl, and NHCOR₈, wherein any of said C₁₋₆ alkyl, OC₁₋₆ alkyl, C₆₋₁₄ aryl, 3-14 membered heterocyclo, C(═O)C₆₋₁₄ aryl, 3-14 membered C(═O)heterocyclo, O—C(═O)C₆₋₁₄ aryl, 3-14 membered O—C(═O)heterocyclo, OC₆₋₁₄ aryl, 3-14 membered O-heterocyclo, C₇₋₂₄ arylalkyl, C(═O)C₇₋₂₄ arylalkyl, O—C(═O)C₇₋₂₄ arylalkyl, O—C₇₋₂₄ arylalkyl, C₂₋₂₀ alkenyl or C₂₋₂₀ alkynyl can optionally be substituted with up to three substituents selected from the group consisting of halogen, C₁₋₆ alkyl, OC₁₋₆ alkyl and CN;
L is CO₂H, an ester thereof, or a pharmaceutically acceptable acid mimetic;
Y is O, (CR₃R₄)ₚ or NR₅;
n′ is 0 or 1;
p is 1 to 3;
X is hydrogen, OH, OR₃, OC₁₋₆ alkyl, OC(═O)C₆₋₁₄ aryl, OC(═O)C₁₋₁₆ alkyl, OC(═O)OC₁₋₆ alkyl, or NR₃R₃;
each R₁, R₃, R_{3′} and R₄ is independently selected from the group consisting of hydrogen, C₁₋₆ alkyl, C₁₋₆ perhaloalkyl, OC₁₋₆ alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, OSO₃R₆, SO₃R₆, PO₃R₆R₇, (CH₂)ₙSO₂NR₈R₉, (CH₂)ₙC(═O)NR₈R₉, NR₈R₉, C(═O)R₁₂, C₆₋₁₄ aryl, 3-14 membered heterocyclo, C(═O)C₆₋₁₄ aryl, 3-14 membered C(═O)heterocyclo, OC(═O)C₆₋₁₄aryl, 3-14 membered OC(═O)heterocyclo, OC₆₋₁₄ aryl, 3-14 membered Oheterocyclo, C₇₋₂₄arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, and NHCOR₈, wherein any of said C₁₋₆ alkyl, OC₁₋₆ alkyl, C₆₋₁₄ aryl, 3 to 14 membered heterocyclo, C(═O)C₆₋₁₄ aryl, 3-14 membered C(═O)heterocyclo, O—C(═O)C₆₋₁₄ aryl, 3-14 membered O—C(═O)heterocyclo, O—C₆₋₁₄ aryl, 3-14 membered O-heterocyclo, C₇₋₂₄arylalkyl, C(═O)C₇₋₂₄ arylalkyl, O—C(═O)C₇₋₂₄ arylalkyl, O—C₇₋₂₄ arylalkyl, C₂₋₂₀ alkenyl or C₂₋₂₀ alkynyl can optionally be substituted with up to three substituents selected from the group consisting of halogen, C₁₋₆ alkyl, OC₁₋₆ alkyl and CN;
each R₆ and R₇ is independently selected from the group consisting of hydrogen and C₁₋₆ alkyl that is optionally substituted with up to three substituents selected from the group consisting of OH, CF₃, SH and halogen;
each R₅, R₈ and R₉ is independently selected from the group consisting of hydrogen, C₁₋₆ alkyl, C₁₋₆ haloalkyl, C₁₋₆ thioalkyl, OH, (CH₂)ₙOSO₃H, (CH₂)_{I}SO₃R₁₀, (CH₂)ₙCO₂R₁₀, SO₃R₁₀, PO₃R₁₀R₁₁, (CH₂)ₙSO₂(CH₂)ₙNR₁₀R₁₁, (CH₂)ₙCONR₁₀R₁₁, COR₁₀, C₆₋₁₄ aryl, 3-14 membered heterocyclo, C(═O)C₆₋₁₄ aryl, 3-14 membered C(═O)heterocyclo, OC(═O)C₆₋₁₄ aryl, 3-14 membered OC(═O)heterocyclo, OC₆₋₁₄aryl, 3-14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, and C₂₋₂₀ alkynyl, wherein any of said C₁₋₆ alkyl, C₆₋₁₄ aryl, 3-14 membered heterocyclo, C(═O)C₆₋₁₄aryl, 3-14 membered C(═O)heterocyclo, OC(═O)C₆₋₁₄ aryl, 3-14 membered OC(═O)heterocyclo, —OC₆₋₁₄ aryl, 3-14 membered Oheterocyclo, C₇₋₂₄arylalkyl, C(═O)C₇₋₂₄arylalkyl, OC(═O)C₇₋₂₄arylalkyl, OC₇₋₂₄ arylalkyl, C₂₋₂₀ alkenyl or C₂₋₂₀ alkynyl can optionally be substituted with up to three substituents selected from the group consisting of halogen, C₁₋₆ alkyl, OC₁₋₆alkyl and CN;
each n is an independently selected integer from 0 to 6;
each I is an independently selected integer from 1 to 6;
each R₁₀ and R₁₁ is independently selected from the group consisting of hydrogen and C₁₋₆ alkyl that is optionally substituted with up to three substituents selected from the group consisting of OH, CF₃, SH and halogen;
each R₁₂ is independently selected from the group consisting of hydrogen, C₁₋₆alkyl, C₁₋₆ perhaloalkyl, OC₁₋₆ alkyl, OC₁ perhaloalkyl, C₁₋₆ thioalkyl, OH, (CH₂)_{I}OSO₃H, (CH₂)_{I}SO₃H, (CH₂)CO₂, (CH₂)_{I}SO₂NR₈R₉, (CH₂)ₙC(═O)NR₈R₉, NR₈R₉, C₂₋₂₀ alkenyl, C₂₋₂₀ alkynyl, or NHCOR₈, wherein any of said C₁₋₆ alkyl, OC₁₋₆ alkyl, C₂₋₂₀ alkenyl or C₂₋₂₀ alkynyl can optionally be substituted with up to three substituents selected from the group consisting of halogen, C₁₋₆alkyl, OC₁₋₆ alkyl and CN; and
Z is C₆₋₁₄ aryl, C₇₋₂₄ arylalkyl, 5 to 13 membered heteroaryl or 3 to 14 membered heterocyclo, wherein each of said C₆₋₁₄aryl, C₇₋₂₄ arylalkyl, 5 to 13 membered heteroaryl and 3 to 14 membered heterocyclo is optionally substituted;
or a pharmaceutically acceptable salt, hydrate or ester thereof.

2. A method of treating scleritis, a scleritis symptom or a scleritis-related condition, the method comprising administering to a subject a therapeutically effective amount of a compound having the Formula III: 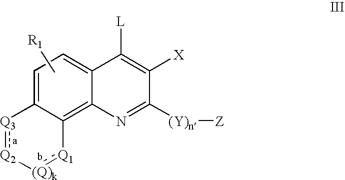
wherein:
bond a and bond b can each independently be a single bond or a double bond;
Q₁, Q₂, Q₃ and Q are each independently CR_{2′}, CHR_{2′}, N or NR₁₃;
k is 0 or 1;
each R₂ is independently selected from the group consisting of hydrogen, C₁₋₆ alkyl, C₁₋₆ perhaloalkyl, OC₁₋₆alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, OSO₃R₆, SO₃R₆, PO₃R₆R₇, (CH₂)_{I}SO₂NR₈R₉, (CH₂)ₙC(═O)NR₈R₉, NR₈R₉, C(═O)R₁₂, C₆₋₁₄ aryl, 3 to 14 membered heterocyclo, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, and NHCOR₈, wherein any of said C₁₋₆alkyl, OC₁₋₆alkyl, C₆₋₁₄aryl, 3 to 14 membered heterocyclo, C(═O)C₆₋₁₄aryl, 3 to 14 membered C(═O)heterocyclo, O—C(═O)C₆₋₁₄aryl, 3 to 14 membered O—C(═O)heterocyclo, OC₆₋₁₄aryl, 3 to 14 membered O-heterocyclo, C₇₋₂₄arylalkyl, C(═O)C₇₋₂₄arylalkyl, O—C(═O)C₇₋₂₄ arylalkyl, O—C₇₋₂₄arylalkyl, C₂₋₂₀alkenyl or C₂₋₂₀alkynyl can optionally be substituted with up to three substituents selected from the group consisting of halogen, C₁₋₆alkyl, OC₁₋₆alkyl and CN;
each R₁₃ is each independently selected from the group consisting of hydrogen, C(═O)R₂₀, SO₂R₂₀, C₁₋₆alkyl, C₁₋₆haloalkyl, C₁₋₆ thioalkyl, OH, (CH₂)ₙSO₃H, (CH₂)_{I}SO₃R₁₀, (CH₂)ₙCO₂R₁₀, SO₃R₁₀, PO₃R₁₀R₁₁, (CH₂)ₙSO₂(CH₂)ₙNR₁₀R₁₁, (CH₂)ₙCONR₁₀R₁₁, COR₁₀, C₆₋₁₄aryl, 3 to 14 membered heterocyclo, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, and C₂₋₂₀ alkynyl, wherein any of said C₁₋₆alkyl, C₆₋₁₄aryl, 3 to 14 membered heterocyclo, C(═O)C₆₋₁₄ aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀alkenyl or C₂₋₂₀alkynyl can optionally be substituted with up to three substituents selected from the group consisting of halogen, C₁₋₆ alkyl, OC₁₋₆ alkyl and CN;
each R₂₀ is independently selected from the group consisting of C₁₋₁₀ alkyl, OC₁₋₁₀ alkyl and NR₆R₇;
L is CO₂H, an ester thereof, or a pharmaceutically acceptable acid mimetic;
Y is O, (CR₃R₄)ₚ or NR₅;
n′ is 0 or 1;
p is 1 to 3;
X is hydrogen, OH, OR₃, OC₁₋₆alkyl, OC(═O)C₆₋₁₄aryl, OC(═O)C₁, alkyl, OC(═O)OC₁₋₆ alkyl, or NR₃R₃;
each R₁, R₃, R_{3′} and R₄ is independently selected from the group consisting of hydrogen, C₁₋₆ alkyl, C₁₋₆ perhaloalkyl, OC₁₋₆ alkyl, OC₁₋₆ perhaloalkyl, halogen, C₁₋₆ thioalkyl, CN, OH, SH, (CH₂)ₙOSO₃H, (CH₂)ₙSO₃H, (CH₂)ₙCO₂R₆, OSO₃R₆, SO₃R₆, PO₃R₆R₇, (CH₂)ₙSO₂NR₈R₉, (CH₂)ₙC(═O)NR₈R₉, NR₉R₉, C(═O)R₁₂, C₆₋₁₄ aryl, 3-14 membered heterocyclo, C(═O)C₆₋₁₄ aryl, 3-14 membered C(═O)heterocyclo, OC(═O)C₆₋₁₄aryl, 3-14 membered OC(═O)heterocyclo, OC₆₋₁₄ aryl, 3-14 membered Oheterocyclo, C₇₋₂₄arylalkyl, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, and NHCOR₈, wherein any of said C₁₋₆ alkyl, C₁₋₆ alkyl, C₆₋₁₄ aryl, 3 to 14 membered heterocyclo, C(═O)C₆₋₁₄ aryl, 3-14 membered C(═O)heterocyclo, O—C(═O)C₆₋₁₄ aryl, 3-14 membered O—C(═O)heterocyclo, O—C₆₋₁₄ aryl, 3-14 membered O-heterocyclo, C₇₋₂₄arylalkyl, C(═O)C₇₋₂₄ arylalkyl, O—C(═O)C₇₋₂₄ arylalkyl, O—C₇₋₂₄ arylalkyl, C₂₋₂₀ alkenyl or C₂₋₂₀ alkynyl can optionally be substituted with up to three substituents selected from the group consisting of halogen, C₁₋₆alkyl, OC₁₋₆ alkyl and CN;
each R₆ and R₇ is independently selected from the group consisting of hydrogen and C₁₋₆ alkyl that is optionally substituted with up to three substituents selected from the group consisting of OH, CF₃, SH and halogen;
each R₅, R₈ and R₉ is independently selected from the group consisting of hydrogen, C₁₋₆ alkyl, C₁₋₆ haloalkyl, C₁₋₆ thioalkyl, OH, (CH₂)ₙOSO₃H, (CH₂)_{I}SO₃R₁₀, (CH₂)ₙCO₂R₁₀, SO₃R₁₀, PO₃R₁₀R₁₁, (CH₂)ₙSO₂(CH₂)ₙNR₁₀R₁₁, (CH₂)ₙCONR₁₀R₁₁, COR₁₀, C₆₋₁₄ aryl, 3-14 membered heterocyclo, C(═O)C₆₋₁₄ aryl, 3-14 membered C(═O)heterocyclo, OC(═O)C₆₋₁₄aryl, 3-14 membered OC(═O)heterocyclo, OC₆₋₁₄ aryl, 3-14 membered Oheterocyclo, C₇₋₂₄ arylalkyl, C(═O)C₇₋₂₄ arylalkyl, OC(═O)C₇₋₂₄ arylalkyl, OC₇₋₂₄ arylalkyl, C₂₋₂₀ alkenyl, and C₂₋₂₀ alkynyl, wherein any of said C₁₋₆ alkyl, C₆₋₁₄ aryl, 3-14 membered heterocyclo, C(═O)C₆₋₁₄ aryl, 3-14 membered C(═O)heterocyclo, OC(═O)C₆₋₁₄ aryl, 3-14 membered OC(═O)heterocyclo, —OC₆₋₁₄ aryl, 3-14 membered Oheterocyclo, C₇₋₂₄arylalkyl, C(═O)C₇₋₂₄arylalkyl, OC(═O)C₇₋₂₄arylalkyl, OC₇₋₂₄ arylalkyl, C₂₋₂₀ alkenyl or C₂₋₂₀ alkynyl can optionally be substituted with up to three substituents selected from the group consisting of halogen, C₁₋₆ alkyl, OC₁₋₆alkyl and CN;
each n is an independently selected integer from 0 to 6;
each I is an independently selected integer from 1 to 6;
each R₁₀ and R₁₁ is independently selected from the group consisting of hydrogen and C₁₋₆ alkyl that is optionally substituted with up to three substituents selected from the group consisting of OH, CF₃, SH and halogen;
each R₁₂ is independently selected from the group consisting of hydrogen, C₁₋₆ alkyl, C16 perhaloalkyl, OC₁₋₆ alkyl, OC₁₋₆ perhaloalkyl, C16 thioalkyl, OH, (CH₂)_{I}OSO₃H, (CH₂)_{I}SO₃H, (CH₂)_{I}CO₂R₆, (CH₂)_{I}SO₂NR₈R₉, (CH₂)_{I}C(═O)NR₈R₉, NR₈R₉, C₂₋₂₀ alkenyl, C₂₋₂₀ alkynyl, or NHCOR₈, wherein any of said C₁₋₆ alkyl, OC₁₋₆ alkyl, C₂₋₂₀ alkenyl or C₂₋₂₀ alkynyl can optionally be substituted with up to three substituents selected from the group consisting of halogen C₁₋₆ alkyl, OC₁₋₆alkyl and CN; and
Z is C₆₋₁₄ aryl, C₇₋₂₄ arylalkyl, 5 to 13 membered heteroaryl or 3 to 14 membered heterocyclo, wherein each of said C₆₋₁₄ aryl, C₇₋₂₄ arylalkyl, 5 to 13 membered heteroaryl and 3 to 14 membered heterocyclo is optionally substituted;
or a pharmaceutically acceptable salt, hydrate or ester thereof.

3. The method of claim 2, wherein k is 1, bonds a and b are each single bonds, and Q, Q₁, Q₂ and Q₃ are each CH₂.

4. The method of claim 2, wherein k is 0, bond a is a single bond, and Q₁, Q₂ and Q₃ are each CH₂.

5. The method of claim 2, wherein k is 0, bond a is a single bond, Q₁ is NH and Q₂ and Q₃ are each CH₂.

6. The method of claim 2, wherein k is 1, bond a and bond b are each double bonds, and Q, Q₁, Q₂ and Q₃ are each CH.

7. The method of claim 2, wherein k is 1, Q₁, Q₂ and Q₃ each are CH₂, and Q is NH.

8. The method of claim 2, wherein n′ is 0.

9. The method of claim 2, wherein n′ is 1, and Y is CR₃R₄.

10. The method of claim 2, wherein X is OH.

11. The method of claim 2, wherein L is CO₂H or an ester thereof.

12. The method of claim 2, wherein Z is selected from:
(a) a five-membered heterocyclic ring containing one to three ring heteroatoms selected from N, S or O; wherein said five-membered heterocyclic ring is optionally substituted by from 1 to 3 substituents selected from halogen, C₁₋₁₀ alkyl, OC₁₋₁₀ alkyl, NO₂, NH₂, CN, CF₃, and CO₂H;
(b) a six-membered heterocyclic ring containing one to three ring heteroatoms selected from N, S or O; wherein said six-membered heterocyclic ring is optionally substituted by from 1 to 3 substituents selected from halogen, C₁₋₁₀ alkyl, OC₁₋₁₀ alkyl, CHO, CO₂H, C(═O)R₂₀, SO₂R₂₀, NO₂, NH₂, CN, CF₃ and OH;
(c) a bicyclic ring moiety having 8 to 14 ring members, and optionally containing from 1 to 3 ring heteroatoms selected from N or O; wherein said bicyclic ring moiety is optionally substituted by from 1 to 3 substituents selected from halogen, C₁₋₆ alkyl, OC₁₋₆ alkyl, CHO, NO₂, NH₂, CN, CF₃, CO₂H, C(═O)R₂₀, SO₂R₂₀, and OH; and
(d) a benzyl, naphthyl, or phenyl ring, each of which is optionally substituted by from 1 to 3 substituents selected from halogen, C₁₋₆ alkyl, phenyl, benzyl, Ophenyl, Obenzyl, SO₂NH₂, SO₂NH(C₁₋₆ alkyl), SO₂N(C₁₋₆ alkyl)₂, CH₂COOH, CO₂H, CO₂Me, CO₂Et, CO₂iPr, C(═O)NH₂, C(═O)NH(C₁₋₆ alkyl), C(═O)N(C₁₋₆ alkyl)₂, OH, SC₁₋₆ alkyl, OC₁₋₆ alkyl, NO₂, NH₂, CF₃, and CN.

13. The method of claim 2, wherein R₁ and each R₂ independently are selected from hydrogen, C₁₋₆ alkyl, C₁₋₆ perhaloalkyl, OC₁₋₆ alkyl, OC₁₋₆ perhaloalkyl, halogen, thioalkyl, CN, OH, SH, (CH₂)ₙOSO₃H, (CH₂)ₙSO₃H, (CH₂)ₙCO₂R₆, OSO₃R₆, SO₃R₆, PO₃R₆R₇, (CH₂)ₙSO₂NR₈R₉, (CH₂)ₙC(═O)NR₈R₉, NR₈R₉, C₆₋₁₄ aryl, 3 to 14 membered heterocyclo, C(═O)R₁₂, C(═O)C₆₋₁₄aryl, 3 to 14 membered C(═O)heterocyclo, OC(═O)C₆₋₁₄aryl, 3 to 14 membered OC(═O)heterocyclo, OC₆₋₁₄aryl, 3 to 14 membered Oheterocyclo, C(═O)C₇₋₂₄ arylalkyl, OC(═O)C₇₋₂₄ arylalkyl, OC₇₋₂₄ arylalkyl, C₂₋₂₀ alkenyl, C₂₋₂₀ alkynyl, and NHCOR₈.

14. The method of claim 2, wherein Z is phenyl or a substituted phenyl.

15. The method of claim 2, wherein the compound has the Formula IV: 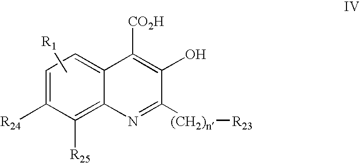
wherein:
n′ is 0 or 1;
R₁ is H, halogen, OH, CN, SH, C₁₋₆ alkyl, OC₁₋₆ alkyl, C₁ perhaloalkyl, C₁₋₆ thioalkyl, C₆₋₁₄ aryl or 5 to 13 membered heteroaryl;
wherein said C₆₋₁₄aryl and said 5 to 13 membered heteroaryl can each optionally be substituted with up to three substituents selected from the group consisting of halogen, OH, CN, SH, NH₂, C₁₋₆ alkyl, OC₁₋₆ alkyl, C₁₋₆ perhaloalkyl and C₁₋₆ thioalkyl; and
wherein said C₁₋₆ alkyl, OC₁₋₆ alkyl and C₁₋₆ thioalkyl can each optionally be substituted with up to three substituents selected from the group consisting of halogen, OH, CN, SH, NH₂, OC₁₋₆ alkyl, C₁, perhaloalkyl and C₁₋₆ thioalkyl;
R₂₃ is C₆₋₁₄ aryl or 5 to 13 membered heteroaryl, wherein said C₆₋₁₄ aryl and said 5 to 13 membered heteroaryl can each optionally be substituted with up to three substituents selected from the group consisting of halogen, OH, CN, SH, NH₂, C₁₋₆ alkyl, OC₁₋₆ alkyl, C₁₋₆ perhaloalkyl and C₁₋₆ thioalkyl; and
R₂₄ and R₂₅ together form —(CH₂)₃—, —(CH₂)₄—, —(CH₂)₂—NH—, —(CH₂)₂—NH—CH₂— or —CH═CH—CH═CH—, wherein up to three hydrogens on the same or different atom(s) may independently be replaced with halogen, OH, CN, SH, NH₂, OC₁₋₆ alkyl, C₁₋₆ perhaloalkyl, C(═O)R₂₀, SO₂R₂₀, or C₁₋₆ thioalkyl; or
a pharmaceutically acceptable salt, hydrate or ester thereof.

16. The method of claim 15, wherein R₂₃ is phenyl or substituted phenyl.

17. The method of claim 15, wherein R₂₃ is phenyl substituted at the 4-position by a substituent selected from F, Cl, Br, OH, CN, SH, NH₂, CH₃, OCH₃, CF₃, and OCF₃.

18. The method of claim 15, wherein R₂₄ and R₂₅ together form unsubstituted —(CH₂)₃—, —(CH₂)₄—, —(CH₂)₂—NH— or —CH═CH—CH═CH—.

19. The method of claim 15, wherein R₁ is H.

20. The method of claim 15, wherein R₁ is H, and R₂₄ and R₂₅ together form —(CH₂)₄—.

21. The method of claim 2, wherein the compound is selected from:
(a) 2-(4-Chloro-phenyl)-3-hydroxy-benzo[h]quinoline-4-carboxylic acid;
(b) 2-(4-Chloro-phenyl)-3-hydroxy-7,8,9,10-tetrahydro-benzo[h]quinoline-4-carboxylic acid;
(c) 3-Hydroxy-2-(4-trifluoromethoxy-benzyl)-7,8,9,10-tetrahydro-benzo[h]quinoline-4-carboxylic acid;
(d) 8-(4-Chloro-benzyl)-7-hydroxy-2,3-dihydro-1H-9-aza-cyclopenta[a]naphthalene-6-carboxylic acid;
(e) 8-(4-Chloro-benzyl)-7-hydroxy-2,3-dihydro-1H-pyrrolo[3,2-h]quinoline-6-carboxylic acid;
(f) 2-(4-Chloro-benzyl)-3-hydroxy-7,8,9,10-tetrahydro-benzo[h]quinoline-4-carboxylic acid;
(g) Triethylammonium 7,8-benzo-2-(4-chlorophenyl)-3-hydroxyquinoline-4-carboxylate;
(h) 2-(3,4-Dichlorobenzyl)-3-hydroxy-7,8,9,10-tetrahydrobenzo[h]quinoline-4-carboxylic acid;
(i) 3-Hydroxy-2-(thiophen-2-ylmethyl)-7,8,9,10-tetrahydrobenzo[h]quinoline-4-carboxylic acid;
(j) 2-(Benzo[b]thiophen-3-ylmethyl)-3-hydroxy-7,8,9,10-tetrahydrobenzo[h]quinoline-4-carboxylic acid;
(k) 2-(2-Chlorobenzyl)-3-hydroxy-7,8,9,10-tetrahydrobenzo[h]quinoline-4-carboxylic acid;
(l) 2-(3-Chlorobenzyl)-3-hydroxy-7,8,9,10-tetrahydrobenzo[h]quinoline-4-carboxylic acid;
(m) 3-Hydroxy-2-[2-(3-methylbenzo[b]thiophen-2-ylmethyl)]-7,8,9,10-tetrahydrobenzo[h]quinoline-4-carboxylic acid;
(n) 3-Hydroxy-2-(thiophen-3-ylmethyl)-7,8,9,10-tetrahydrobenzo[h]quinoline-4-carboxylic acid;
(o) 3-Hydroxy-2-(indol-3-ylmethyl)-7,8,9,10-tetrahydrobenzo[h]quinoline-4-carboxylic acid;
2-(5-Chlorobenzo[b]thiophen-3-ylmethyl)-3-hydroxy-7,8,9,10-tetrahydrobenzo[h]quinoline-4-carboxylic acid;
(p) 3-Hydroxy-2-phenyl-7,8,9,10-tetrahydro-benzo[h]quinoline-4-carboxylic acid;
(q) 2-(4-Cyano-benzyl)-3-hydroxy-7,8,9,10-tetrahydro-benzo[h]quinoline-4-carboxylic acid;
(r) 2-(4-Carboxy-benzyl)-3-hydroxy-7,8,9,10-tetrahydro-benzo[h]quinoline-4-carboxylic acid;
(s) 2-(4-Carbamoyl-benzyl)-3-hydroxy-7,8,9,10-tetrahydro-benzo[h]quinoline-4-carboxylic acid;
(t) 2-Benzyl-3-hydroxy-7,8,9,10-tetrahydro-benzo[h]quinoline-4-carboxylic acid;
(u) 3-Hydroxy-2-phenethyl-7,8,9,10-tetrahydro-benzo[h]quinoline-4-carboxylic acid;
(v) 2-(4-Chloro-benzyl)-3-hydroxy-7,8,9,10-tetrahydro-[1,9]phenanthroline-4-carboxylic acid;
(w) 2-(4-Chloro-benzyl)-3-hydroxy-9-isopropyl-7,8,9,10-tetrahydro-[1,9]phenanthroline-4-carboxylic acid;
(x) 9-Benzyl-2-(4-chloro-benzyl)-3-hydroxy-7,8,9,10-tetrahydro-[1,9]phenanthroline-4-carboxylic acid;
(y) 2-(4-Chloro-benzyl)-9-ethyl-3-hydroxy-7,8,9,10-tetrahydro-[1,9]phenanthroline-4-carboxylic acid;
(z) 9-Acetyl-2-(4-chloro-benzyl)-3-hydroxy-7,8,9,10-tetrahydro-[1,9]phenanthroline-4-carboxylic acid;
(aa) 9-Carbamoyl-2-(4-chloro-benzyl)-3-hydroxy-7,8,9,10-tetrahydro-[1,9]phenanthroline-4-carboxylic acid;
(bb) 9-Benzoyl-2-(4-chloro-benzyl)-3-hydroxy-7,8,9,10-tetrahydro-[1,9]phenanthroline-4-carboxylic acid;
(cc) 9-Benzoyl-3-benzoyloxy-2-(4-chloro-benzyl)-7,8,9,10-tetrahydro-[1,9]phenanthroline-4-carboxylic acid;
(dd) 2-(4-Chloro-benzyl)-3-hydroxy-9-methanesulfonyl-7,8,9,10-tetrahydro-[1,9]phenanthroline-4-carboxylic acid;
(ee) 2-(4-Chloro-benzyl)-3-hydroxy-7,10-dihydro-8H-[1,9]phenanthroline-4,9-dicarboxylic acid 9-ethyl ester;
(ff) 2-(4-Chloro-benzyl)-3-ethoxycarbonyloxy-7,10-dihydro-8H-[1,9]phenanthroline-4,9-dicarboxylic acid 9-ethyl ester;
(gg) 2-(4-Chloro-benzyl)-3-hydroxy-9-phenylacetyl-7,8,9,10-tetrahydro-[1,9]phenanthroline-4-carboxylic acid; and
(hh) 2-(4-Chloro-benzyl)-3-hydroxy-9-(propane-2-sulfonyl)-7,8,9,10-tetrahydro-[1,9]phenanthroline-4-carboxylic acid;
(ii) 2-(4-Chloro-benzyl)-3-methoxy-7,8,9,10-tetrahydro-benzo[h]quinoline-4-carboxylic acid;
(jj) 3-Hydroxy-2-piperidin-4-yl-7,8,9,10-tetrahydro-benzo[h]quinoline-4-carboxylic acid;
(kk) 2-(1-acetyl-piperidin-4-yl)-3-hydroxy-7, 8,9,10-tetrahydro-benzo[h]quinoline-4-carboxylic acid.

23. A pharmaceutical composition for the treatment of scleritis, a scleritis-related disorder or a scleritis symptom, the pharmaceutical composition comprising a compound of Formula I, III or IV, or a pharmaceutically acceptable salt, hydrate or ester thereof, and a pharmaceutically acceptable carrier or excipient, wherein the compounds of Formula I, III or IV are as defined herein.

24. A method for reducing or preventing leukocyte adhesion to the vascular endothelium comprising administering to a subject a therapeutically effective amount of a compound of Formula I, III or IV, or a pharmaceutically acceptable salt, hydrate or ester thereof.
